(19) United States
(12) Patent Application Publication
(10) Pub. No.: US 2020/0171030 A1
(43) Pub. Date: Jun. 4, 2020

(54) DUAL INHIBITORS OF ALK5 AND P38A MAP KINASE

(71) Applicant: INTEGRAL BIOSCIENCES PRIVATE LIMITED, Noida (IN)

(72) Inventors: Sarvajit CHAKRAVARTY, Edmond, OK (US); Dhananjay PENDHARKAR, Aundh (IN); Dilip V. JARIKOTE, Wakad (IN); Bhausaheb B. BHAGWAT, Viroli (IN); Anil K. AGARWAL, Panposh (IN); Brahmam PUJALA, Nekkallu-Post (IN); Sreekanth A. RAMACHANDRAN, Noida (IN); Sagar PATNI, Noida (IN)

(21) Appl. No.: 16/632,213

(22) PCT Filed: Jan. 17, 2019
(86) PCT No.: PCT/IB19/50391

§ 371 Date: Jan. 17, 2020

(30) Foreign Application Priority Data
Jan. 18, 2018 (IN) 201711025533

(51) Int. Cl. A61K 31/506; A61K 31/5377; A61K 31/444; C07D 471/04; A61K 45/06

(52) U.S. Cl. CPC A61K 31/506; A61K 31/5377; A61K 45/06; C07D 471/04; A61K 31/444

(57) ABSTRACT

The present disclosure generally relates to the compounds that exhibits protein kinase inhibitory activity. Specifically, the present disclosure provides compounds of formula (I) that exhibits dual inhibitory activity against ALK5 and P38A MAP kinase. The present disclosure also provides process(es) for preparation of such compounds, pharmaceutical compositions containing one or a combination of these compounds, and methods of treatment of conditions associated with excessive activity of any or a combination of transforming growth factor-beta (TGFβ) and p38 mitogen-activated protein kinase (MAPK) utilizing these compounds. An aspect of the present disclosure provides compounds of Formula (I):

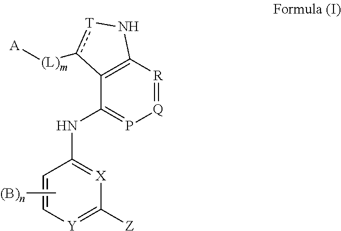

and pharmaceutically acceptable salt, polymorph, solvate or stereoisomer thereof that exhibit dual inhibitory activity against ALK5 and P38 alpha.

### FIELD OF THE INVENTION

The present disclosure generally relates to the compounds that exhibits protein kinase inhibitory activity. Specifically, the present disclosure provides compounds of Formula (IA) that exhibits dual inhibitory activity against ALK5 and P38α MAP kinase. The present disclosure also provides process(es) for preparation of such compounds, pharmaceutical compositions containing one or a combination of these compounds, and methods of treatment of conditions associated with excessive activity of any or a combination of transforming growth factor-beta (TGFβ) and p38 mitogen-activated protein kinase (MAPK) utilizing these compounds.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

TGFβ belongs to a superfamily of multifunctional proteins that include, for example, TGFβ1, TGFβ2, and TGFβ3, which are pleiotropic modulators of cell growth and differentiation, embryonic and bone development, extracellular matrix formation, hematopoiesis, and immune and inflammatory responses. For instance, TGFβ1 inhibits the growth of many cell types, including epithelial cells, but stimulates the proliferation of various types of mesenchymal cells. Other members of this superfamily include activin, inhibin, bone morphogenic protein, and Mullerian inhibiting substance. The members of the TGFβ family initiate intracellular signaling pathways leading ultimately to the expression of genes that regulate the cell cycle, control proliferative responses, or relate to extracellular matrix proteins that mediate outside-in cell signaling, cell adhesion, migration and intercellular communication.

Therefore, inhibitors of the TGFβ intracellular signaling pathway are recognized as being useful primarily for the treatment of fibroproliferative diseases. Fibroproliferative diseases include kidney disorders associated with unregulated TGFβ activity and excessive fibrosis including glomerulonephritis (GN), such as mesangial proliferative GN, immune GN, and crescentic GN. Other renal conditions include diabetic nephropathy, renal interstitial fibrosis, renal fibrosis in transplant patients receiving cyclosporine, and HIV-associated nephropathy. Collagen vascular disorders include progressive systemic sclerosis, polymyositis, scleroderma, dermatomyositis, eosinophilic fasciitis, morphea, or those associated with the occurrence of Raynaud's syndrome. Lung fibroses resulting from excessive TGFβ activity include adult respiratory distress syndrome, chronic obstructive pulmonary disease (COPD), idiopathic pulmonary fibrosis, and interstitial pulmonary fibrosis often associated with autoimmune disorders, such as systemic lupus erythematosus and scleroderma, chemical contact, or allergies. Another autoimmune disorder associated with fibroproliferative characteristics is rheumatoid arthritis. Fibroproliferative conditions can be associated with surgical eye procedures. Such procedures include retinal reattachment surgery accompanying proliferative vitreoretinopathy, cataract extraction with intraocular lens implantation, and post glaucoma drainage surgery.

Transforming growth factor-β (TGF-β), a family of cytokines regulates a plethora of cellular processes, including growth control, differentiation, extracellular matrix production, migration, survival, apoptosis and immune suppression. This in part due to their ability to exert direct control over multiple signaling networks in addition to the control of the canonical SMAD-dependent signaling pathway. Some of the SMAD-independent pathways which are influenced by TGF-β ligands include the mitogen-activated protein kinase (MAPK) pathways, the PI3K/AKT/mTOR pathways and the RhoA-dependent signaling pathways. Among the MAPK pathway, the activation of P38 MAPK by TGF-β ligands has been well characterized. p38 mitogen activated protein kinase (p38MAPK) pathway has been shown to contribute in TGF-β-mediated EMT and tumor cell migration. It has also been shown that co-administration of a P38α inhibitor and an ALK5 inhibitor modulated the development of fibrosis in Adriyamicin induced renal fibrosis model via the reduction in active TGF-1 production, reduced myofibroblast accumulation, and decreased expression of collagen type IV and fibronectin. Even though 4 isoforms of P38 MAPK are known, most stimuli result in the activation of P38α by the MAP kinase kinases (MKKs).

The p38 mitogen-activated protein kinase (MAPK) pathway and transforming growth factor TGFβ1/Smad signaling pathway are thus important intracellular signaling pathways involved in many human diseases such as inflammation and cancer. The synergistic value of inhibiting these two pathways has been explored using combination of ALK5 and p38 inhibitors.

In view of above, efficacious dual inhibitors of ALK5 and P38α kinases are needed as potentially valuable therapeutic agents. It is thus desirable to develop new chemical entities which can act as dual ALK5/P38α kinases inhibitors so that more appropriate pharmaceuticals can be selected with fewer side effects.

### OBJECTS OF THE INVENTION

Primary object of the present disclosure is to provide dual ALK5/P38α kinase inhibitor compounds.

Another object of the present disclosure is to provide a method of synthesis of compounds that possess dual ALK5/P38α kinase inhibitory activity.

Another object of the present disclosure is to provide method of treatment of conditions associated with excessive activity of any or a combination of ALK5 and P38α kinase.

Another object of the present disclosure is to provide method of treatment of cancer by administering at least one agent that inhibit any or a combination of ALK5 and P38α MAP kinase.

Another object of the present disclosure is to provide method of treatment of inflammatory disease/ailment by administering at least one agent that inhibit any or a combination of ALK5 and P38α MAP kinase.

Another object of the present disclosure is to provide method of treatment of cancer by administering at least one agent that inhibit any or a combination of ALK5 and P38α MAP kinase in combination with at least one immunotherapeutic agent.

Another object of the present disclosure is to provide method of treatment of inflammatory disease by administering at least one agent that inhibit any or a combination of ALK5 and P38α MAP kinase in combination with at least one anti-inflammatory agent.

Still another object of the present disclosure is to provide pharmaceutical compositions containing at least one compound that inhibit ALK5 and P38α MAP kinase.

Still further object of the present disclosure is to provide pharmaceutical compositions containing at least one compound possessing dual inhibitory activity against ALK5 and P38α MAP kinase that find utility in treatment of cancer.

Still further object of the present disclosure is to provide pharmaceutical compositions containing at least one compound possessing dual inhibitory activity against ALK5 and P38α MAP kinase that find utility in treatment of inflammatory disease.

### SUMMARY OF THE INVENTION

The present disclosure generally relates to the compounds that exhibits protein kinase inhibitory activity. Specifically, the present disclosure provides compounds of formula (I) that exhibits dual inhibitory activity against ALK5 and P38α MAP kinase. The present disclosure also provides process(es) for preparation of such compounds, pharmaceutical compositions containing one or a combination of these compounds, and methods of treatment of conditions associated with excessive activity of any or a combination of transforming growth factor-beta (TGFβ) and p38 mitogen-activated protein kinase (MAPK) utilizing these compounds.

An aspect of the present disclosure provides a compound of Formula (IA) and pharmaceutically acceptable salt, polymorph, solvate or stereoisomer thereof:

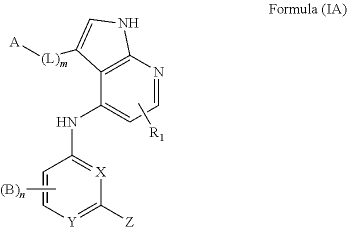

wherein:
L represents —C(O)C(O)—;
X and Y independently represent CR₁ or N;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted C₁₋₆ linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, —OC(O)R₁, —OC(O)NH₂, —OC(O)NHR₁, —OC(O)NR₁R₂, —NO₂, —NH₂, —NHR₁, —NR₁R₂, —N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SH, —SR₁, —S(O)H, —S(O)R₁, —SO₂R₁, —SO₂NH₂, —SO₂NHR₁, —SO₂NR₁R₂, —CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁;
Z represents an optionally substituted aryl or an optionally substituted heteroaryl having up to 12 carbon atoms with one or more heteroatoms selected from O, N and S;
B represents H, halogen, C₁₋₆ linear or branched alkyl, acyl, C₃₋₈ cycloalkyl, C₁₋C₄ haloalkyl, C₁₋C₄ haloalkoxy, CHR₂ₐR_{2b}, —OH, —OR₁, and —OCR₂ₐ, wherein R₂ₐ and R_{2b} independently represent H, C₁₋₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁₋C₄ haloalkyl, C₁-C₄ haloalkoxy, (CH)ₙR₄ₐR_{4b}, —COR₄ₐ, —CONR₄ₐ, —OC(O)R₄ₐ, —OC(O)NHR₄ₐ,
—C(O)CH₂OH, —CH(CH₃)₂OH, and —C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-2; and
m is 0 or 1;
with a proviso that at least one of X and Y represents N and when X represents CR₁, m≠0.

In an embodiment, the compound of Formula (IA) exhibits dual inhibitory activity against ALK5 and P38α MAP kinase.

In an embodiment, the compound is of any of Formula (IIA), Formula (III) or Formula (IV):

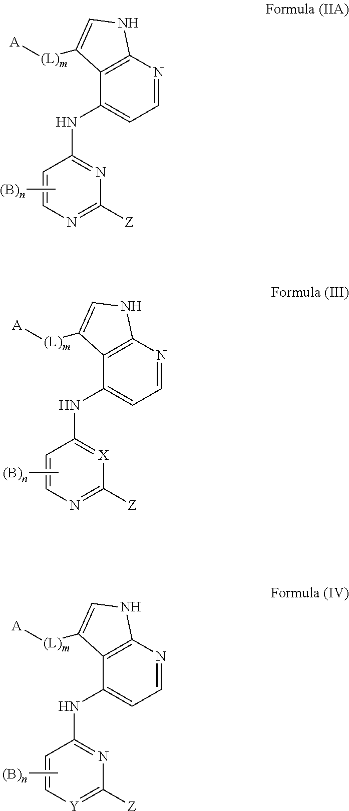

wherein:
L represents —C(O)C(O)—;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁,
—NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R₂, —CONHSO₂H,
—C(O)NHSO₂R₁, —C(O)NR₁SO₂R₁;
X and Y represent CR₁;
Z represents an optionally substituted aryl or an optionally substituted heteroaryl having up to 12 carbon atoms with one or more heteroatoms selected from O, N and S;
B represents H, halogen, C₁₋₆ linear or branched alkyl, acyl, C₃₋₈ cycloalkyl, C_{1-C4} halo alkyl, C₁₋C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-2; and
m is 0 or 1,
with a proviso that when X represents CR₁, m≠0.

In an embodiment, the compound is of any of Formula (IIB) or Formula (IVA) or Formula (IVB):

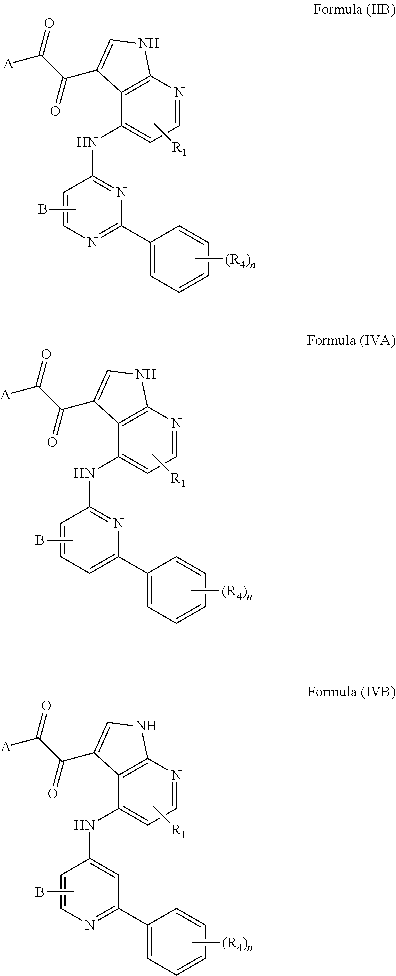

wherein:
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁,
—NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R₂, —CONHSO₂H,
—C(O)NHSO₂R₁, —C(O)NR₁SO₂R₁;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-5.

In an embodiment, the compound is of Formula (V) and pharmaceutically acceptable salt, polymorph, solvate or stereoisomer thereof:

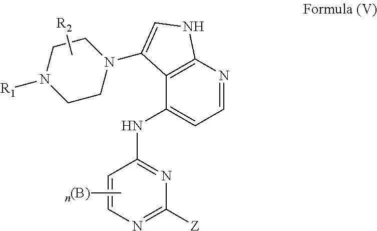

wherein:
R₁ and R₂ is independently H, halogen, —OH, —OR₁, —OCR₁—CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SH, —SR₁, —S(O)H, —S(O)R₁, —SO₂R₁, —SO₂NH₂, —SO₂NHR₁, —SO₂NR₁R₂, CF₃, —CHF₂, —CH₂F, —OCF₃, —OCHF₂, —CN, —CO₂H, —CO₂R1, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁; optionally substituted C₁₋₆ linear or branched alkyl group, an optionally substituted C₂₋₆ linear or branched alkenyl group, an optionally substituted C₂₋₆ alkynyl group; an optionally substituted C₃₋₇ cyclic alkyl group;
Z represents an optionally substituted aryl or an optionally substituted heteroaryl having up to 12 carbon atoms with one or more heteroatoms independently selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, halogen, —OH, —OR₁, —OCR₂ₐ wherein R₂ₐ and R_{2b} are independently hydrogen, C₁-C₆ linear or branched alkyl, C₃₋₈ cycloalkyl, optionally substituted aryl, and an optionally substituted heteroaryl group having up to 12 carbon atoms and having one or more heteroatoms in its ring system which are each independently selected from O, N and S, aryl and hetro aryl may be further substituted with R₄;
R₄ represents C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ, —C(O)CH₂OH, —CH(CH₃)₂OH, C(O)R₄ₐR_{4b}OH, wherein R₄ₐ and R_{4b} are independently hydrogen, C₁-C₆ linear or branched alkyl, C₃₋₈ cycloalkyl, Halo, OH, NH₂, —CONH₂; and
n is 0 or an integer from 1-2.

In an embodiment, the compound is selected from a group consisting of:
N-(2-(4-fluorophenyl)-5-isopropylpyrimidin-4-yl)-3-(piperazin-1-yl)-1H-pyrrolo [2,3-b] pyridin-4-amine;
N-(2-(4-fluorophenyl)-5-isopropylpyrimidin-4-yl)-3-(4-methylpiperazin-1-yl)-1H-pyrrolo [2,3-b] pyridin-4-amine;
N-(2-(4-fluorophenyl)-5-isopropylpyrimidin-4-yl)-3-(piperidin-1-yl)-1H-pyrrolo [2,3-b] pyridin-4-amine;
N-(2-(4-fluorophenyl)-5-isopropylpyrimidin-4-yl)-3-(2-methylpiperazin-1-yl)-1H-pyrrolo[2,3-b]pyridin-4-amine;
N-(2-(4-fluorophenyl)-5-isopropylpyrimidin-4-yl)-3-morpholino-1H-pyrrolo [2,3-b]pyridin-4-amine;
N-(2-(4-fluorophenyl)-5-isopropylpyrimidin-4-yl)-3-(1H-1,2,4-triazol-1-yl)-1H-pyrrolo[2,3-b] pyridin-4-amine;
(S)—N-(2-(4-fluorophenyl)-5-isopropylpyrimidin-4-yl)-3-(2-methylpiperazin-1-yl)-1H-pyrrolo[2,3-b]pyridin-4-amine;
(R)—N-(2-(4-fluorophenyl)-5-isopropylpyrimidin-4-yl)-3-(2-methylpiperazin-1-yl)-1H-pyrrolo[2,3-b]pyridin-4-amine;
(R)—N-(2-(4-fluorophenyl)-5-isopropylpyrimidin-4-yl)-3-(3-methylpiperazin-1-yl)-1H-pyrrolo[2,3-b]pyridin-4-amine;
4-(4-(2-(4-fluorophenyl)-5-isopropylpyrimidin-4-ylamino)-1H-pyrrolo [2,3-b] pyridin-3-yl) piperazin-2-one;
(S)—N-(2-(4-fluorophenyl)-5-isopropylpyrimidin-4-yl)-3-(3-methylpiperazin-1-yl)-1H-pyrrolo[2,3-b]pyridin-4-amine;
1-(4-((2-(4-fluorophenyl)-5-isopropylpyrimidin-4-yl)amino)-1H-pyrrolo[2,3-b]pyridin-3-yl)piperidine-4-carboxamide;
4-(4-((2-(4-fluorophenyl)-5-isopropylpyrimidin-4-yl)amino)-1H-pyrrolo[2,3-b]pyridin-3-yl)piperidin-4-ol;
N-(2-(4-fluorophenyl)-5-isopropylpyrimidin-4-yl)-3-(3-methylmorpholino)-1H-pyrrolo [2,3-b] pyridin-4-amine;
3-(4-aminopiperidin-1-yl)-N-(2-(4-fluorophenyl)-5-isopropylpyrimidin-4-yl)-1H-pyrrolo[2,3-b]pyridin-4-amine;
2-(4-((2-(4-fluorophenyl)-5-isopropylpyrimidin-4-yl) amino)-1H-pyrrolo[2,3-b]pyridin-3-yl)-N,N-dimethyl-2-oxoacetamide;
2-(4-((2-(4-fluorophenyl)-5-isopropylpyrimidin-4-yl)amino)-1H-pyrrolo[2,3-b]pyridin-3-yl)-2-oxoacetamide;
2-(4-((2-(5-chloro-2-fluorophenyl)-5-isopropylpyrimidin-4-yl)amino)-1H-pyrrolo[2,3-b]pyridin-3-yl)-N-methyl-2-oxoacetamide;
2-(4-((2-(4-fluorophenyl)-5-isopropylpyridin-4-yl)amino)-1H-pyrrolo[2,3-b]pyridin-3-yl)-2-oxoacetamide;
N-butyl-2-(4-(2-(4-fluorophenyl)-5-isopropylpyrimidin-4-ylamino)-1H-pyrrolo [2,3-b]pyridin-3-yl)-2-oxoacetamide;
2-(4-((2-(4-fluorophenyl)-5-isopropylpyridin-4-yl)amino)-1H-pyrrolo[2,3-b]pyridin-3-yl)-N-methyl-2-oxoacetamide;
1-(4-(2-(4-fluorophenyl)-5-isopropylpyrimidin-4-ylamino)-1H-pyrrolo [2,3-b] pyridin-3-yl)-2-morpholinoethane-1, 2-dione;
1-(4-(2-(4-fluorophenyl)-5-isopropylpyrimidin-4-ylamino)-1H-pyrrolo [2,3-b] pyridin-3-yl)-2-(4-methylpiperazin-1-yl) ethane-1,2-dione;
1-(4-(2-(4-fluorophenyl)-5-isopropylpyrimidin-4-ylamino)-1H-pyrrolo[2,3-b]pyridin-3-yl)-2-(piperazin-1-yl)ethane-1,2-dione;
2-(4-((5-isopropyl-2-(4-methoxyphenyl) pyrimidin-4-yl) amino)-1H-pyrrolo[2,3-b]pyridin-3-yl)-2-oxoacetamide;
2-(4-((5-isopropyl-2-phenylpyrimidin-4-yl)amino)-1H-pyrrolo[2,3-b]pyridin-3-yl)-2-oxoacetamide;
2-(4-(6-(4-fluorophenyl)-3-isopropylpyridin-2-ylamino)-1H-pyrrolo [2,3-b] pyridin-3-yl)-2-oxoacetamide;
2-(4-((2-(4-fluorophenyl)-5-isopropylpyrimidin-4-yl)amino)-1H-pyrrolo[2,3-b]pyridin-3-yl)-N-methyl-2-oxoacetamide,
or pharmaceutically acceptable salt, polymorph, solvate or stereoisomer thereof.

In an embodiment, said compound or a pharmaceutically acceptable salt, polymorph, solvate or stereoisomer thereof being used for manufacture of a medicament for treatment of a disorder characterized by excessive level of any or a combination of ALK5 and P38α MAP kinase.

### DETAILED DESCRIPTION OF THE INVENTION

The embodiments herein and the various features and advantageous details thereof are explained more comprehensively with reference to the non-limiting embodiments that are detailed in the following description. Descriptions of well-known components and processing techniques are omitted so as to not unnecessarily obscure the embodiments herein.

The examples used herein are intended merely to facilitate an understanding of the ways in which the embodiments herein may be practiced and to further enable those of skill in the ait to practice the embodiments herein. Accordingly, the examples should not be construed as limiting the scope of the embodiments herein.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

As used herein, the terms “comprise”, “comprises”, “comprising”, “include”, “includes”, and “including” are meant to be non-limiting, i.e., other steps and other ingredients which do not affect the end of result can be added. The above terms encompass the terms “consisting of” and “consisting essentially of”.

As used herein, the terms “composition” “blend,” or “mixture” are all intended to be used interchangeably.

The terms “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The present disclosure generally relates to the compounds that exhibits protein kinase inhibitory activity. Specifically, the present disclosure provides compounds of formula (1) that exhibits dual inhibitory activity against ALK5 and P38α MAP kinase. The present disclosure also provides process(es) for preparation of such compounds, pharmaceutical compositions containing one or a combination of these compounds, and methods of treatment of conditions associated with excessive activity of any or a combination of transforming growth factor-beta (TGFβ) and p38 mitogen-activated protein kinase (MAPK) utilizing these compounds.

The present disclosure provides compounds of Formula (I) and pharmaceutically acceptable salt, polymorph, solvate or stereoisomer thereof:

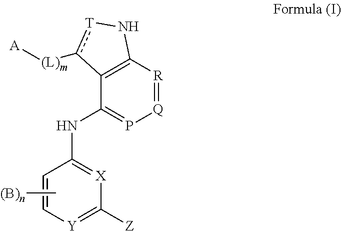

wherein:
L is —C(O)C(O)—;
X and Y independently represent CR₁ or N;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH2, —NHC(O)NR₁R₂, —NR1C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁, or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ is independently H, optionally substituted C₁₋₆ linear or branched alkyl group, an optionally substituted C₂₋₆ linear or branched alkenyl group, an optionally substituted C₂₋₆ alkynyl group and an optionally substituted C₃₋₇ cyclic alkyl group;
R₃ is halogen, oxo, —OH, —OR₁, —OCR₁—CONR₁, OC(O)R₁, —OC(O)NH₂, OC(O)NHR1, —OC(O)NR₁R₂, —NO₂, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SH, —SR₁, —S(O)H, —S(O)R₁, —SO₂R₁, —SO₂NH₂, —SO₂NHR₁, —SO₂NR₁R₂, CF₃, —CHF₂, —CH₂F, —OCF₃, —OCHF₂, —CN, —CO₂H, —CO₂R₁, —CHO, —C(O)R₁, —C(O)NH₂, —C(O)NHR₁, —C(O)NR₁R₂, —CONHSO₂H, —C(O)NHSO2R₁, —C(O)NR₁SO₂R₁;
X, Y, P, Q, R, and T independently represents CR₁, or N;
Z represents an optionally substituted aryl or an optionally substituted heteroaryl having up to 12 carbon atoms with one or more heteroatoms independently selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, halogen, —OH, —OR₁, —OCR₂ₐ, wherein R₂ₐ and R_{2b} are independently hydrogen, C₁-C₆ linear or branched alkyl, C₃₋₈ cycloalkyl, optionally substituted aryl, and an optionally substituted heteroaryl group having up to 12 carbon atoms and having one or more heteroatoms in its ring system which are each independently selected from O, N and S, aryl and hetro aryl may be further substituted with R₄;
R₄ represents C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ, —C(O)CH₂OH, —CH(CH₃)₂OH, C(O)R₄ₐR_{4b}OH, wherein R₄ₐ and R_{4b} are independently hydrogen, C₁-C₆ linear or branched alkyl, C₃₋₈ cycloalkyl, Halo, OH, NH₂, —CONH₂;
n is 0 or an integer from 1-2; and
m is 0 or 1,
with a proviso that at least one of X and Y represents N and when X represents CR1, m≠0.

In an embodiment, the compounds of Formula (I) exhibits dual inhibitory activity against ALK5 and P38α MAP kinase.

An aspect of the present disclosure provides a compound of Formula (IA) and pharmaceutically acceptable salt, polymorph, solvate or stereoisomer thereof:

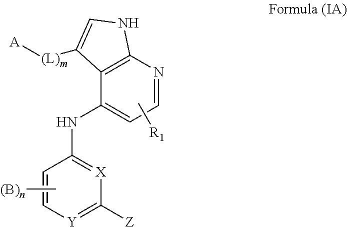

wherein:
L represents —C(O)C(O)—;
X and Y independently represent CR₁ or N;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁,
—NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R₂, —CONHSO₂H,
—C(O)NHSO₂R₁, —C(O)NR₁SO₂R₁;
Z represents an optionally substituted aryl or an optionally substituted heteroaryl having up to 12 carbon atoms with one or more heteroatoms selected from O, N and S;
B represents H, halogen, linear or branched alkyl, acyl, C₃₋₈ cycloalkyl, C₁-C₄ halo alkyl, C₁-C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R;
R₄ represents any of C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-2; and
m is 0 or 1,
with a proviso that at least one of X and Y represents N and when X represents CR₁, m≠0.

The present disclosure provides a dual ALK5 and P38α MAP kinase inhibitor compound of Formula (II) and pharmaceutically acceptable salt, polymorph, solvate or stereoisomer thereof:

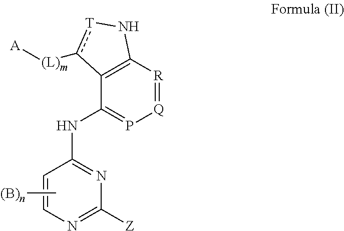

wherein:
L is —C(O)C(O)—;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR1C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁, or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ is independently H, optionally substituted C₁₋₆ linear or branched alkyl group, an optionally substituted C₂₋₆ linear or branched alkenyl group, an optionally substituted C₂₋₆ alkynyl group and an optionally substituted C₃₋₇ cyclic alkyl group;
R₃ is halogen, oxo, —OH, —OR, —OCR₁—CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SH, —SR₁, —S(O)H, —S(O)R₁, —SO₂R₁, —SO₂NH₂, —SO₂NHR₁, —SO₂NR₁R₂, CF₃, —CHF₂, —CH₂F, —OCF₃, —OCHF₂, —CN, —CO₂H, —CO₂R₁, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁;
P, Q, R and T independently represents CR₁, or N;
Z represents an optionally substituted aryl or an optionally substituted heteroaryl having up to 12 carbon atoms with one or more heteroatoms independently selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, halogen, —OH, —OR₁, —OCR₂ₐ, wherein R₂ₐ and R_{2b} are independently hydrogen, C₁-C₆ linear or branched alkyl, C₃₋₈ cycloalkyl, optionally substituted aryl, and an optionally substituted heteroaryl group having up to 12 carbon atoms and having one or more heteroatoms in its ring system which are each independently selected from O, N and S, aryl and hetro aryl may be further substituted with R₄;
R₄ represents C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ, —C(O)CH₂OH, —CH(CH₃)₂OH, C(O)R₄ₐR_{4b}OH, wherein R₄ₐ and R_{4b} are independently hydrogen, C₁-C₆ linear or branched alkyl, C₃₋₈ cycloalkyl, Halo, OH, NH₂, —CONH₂;
n is 0 or an integer from 1-2; and
m is 0 or 1.

In a preferred embodiment, A represents any of —NHR₁, —NR₁R₂,

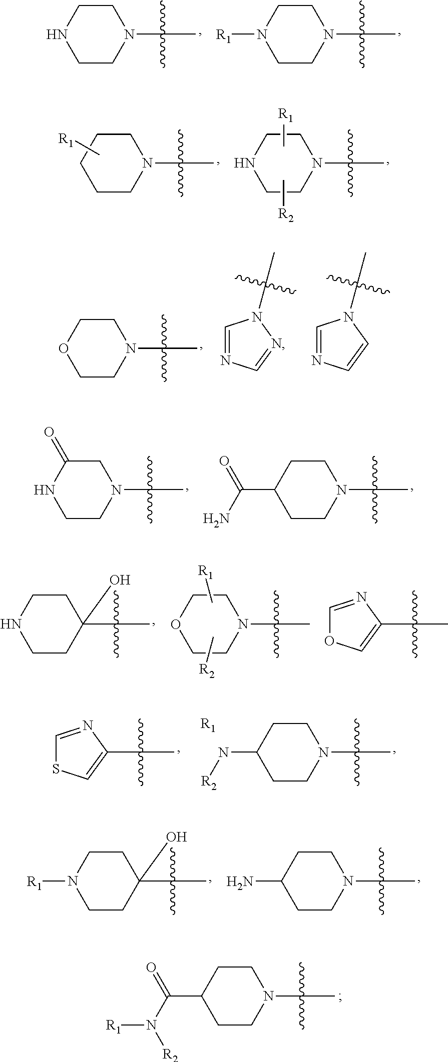

and stereoisomer(s) thereof, and wherein m is 0.

In a preferred embodiment, A represents any of —NHR₁, —NR₁R₂,

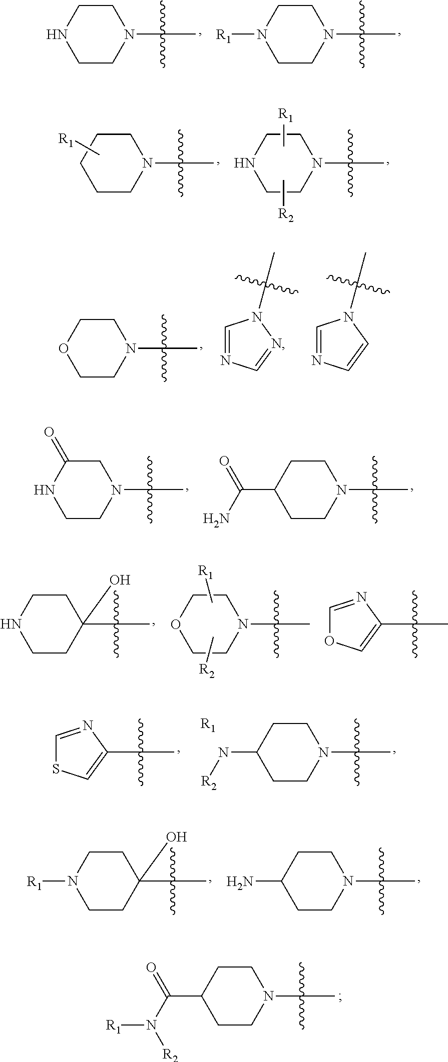

and stereoisomer(s) thereof, and wherein m is 1.

In an embodiment, the compound is of any of Formula (IIA), Formula (III) or Formula (IV):

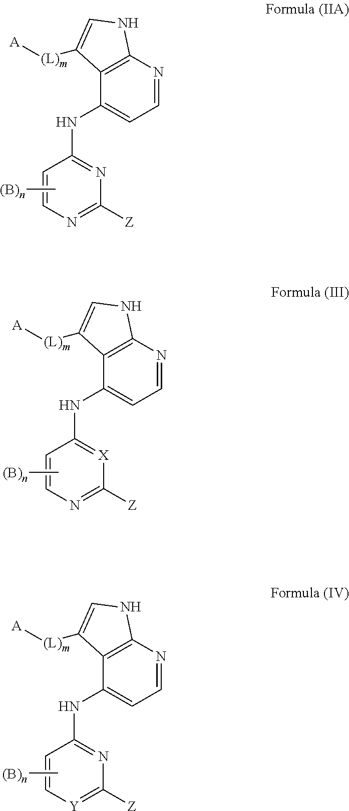

wherein:
L represents —C(O)C(O)—;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂RJ, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted C₁₋₆ linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁,
—NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R₂, —CONHSO₂H,
—C(O)NHSO₂R₁, —C(O)NR₁SO₂R₁;
X and Y represent CR₁;
Z represents an optionally substituted aryl or an optionally substituted heteroaryl having up to 12 carbon atoms with one or more heteroatoms selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁-C₄ halo alkyl, C₁-C₄ halo alkoxy. (CH)ₙR₄ₐR_{4b}, —COR₄ₐ, CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-2; and
m is 0 or 1,
with a proviso that when X represents CR₁, m≠0.

In an embodiment, the compound is of any of Formula (IIB) or Formula (IVA) or Formula (IVB):

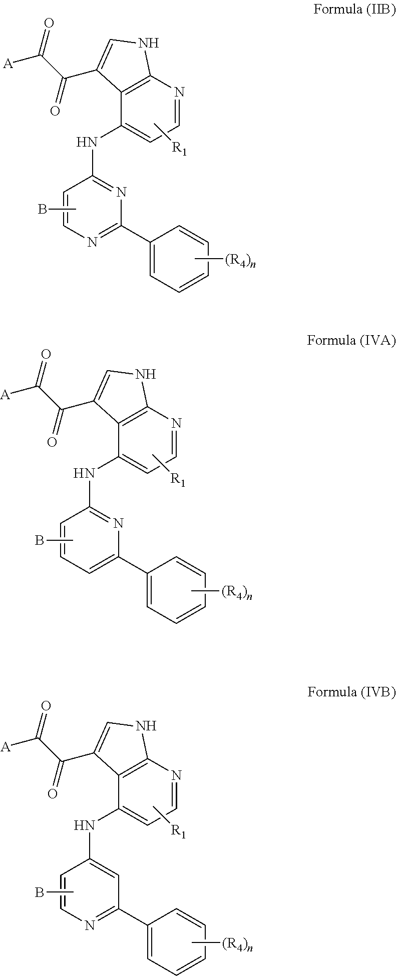

wherein:
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted C₁₋₆ linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R, —NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN. —COOH, —COOR₁, —CHO, —C(O)R₁, —C(O)NH₂, —C(O)NHR₁, —C(O)NR₁R₂, —CONHSO₂H,
—C(O)NHSO₂R₁, —C(O)NR₁SO₂R₁;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁-C₄ halo alkyl, C₁-C₄ halo alkoxy. (CH)ₙR₄ₐR_{4b}, —COR₄ₐ, —CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-5.

The present disclosure provides a dual ALK5 and P38α MAP kinase inhibitor compound of Formula (V) and pharmaceutically acceptable salt, polymorph, solvate or stereoisomer thereof:

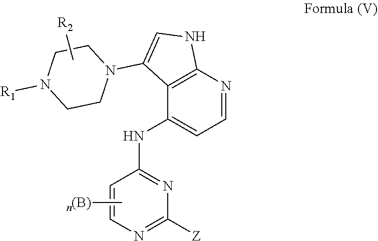

wherein:
R₁ and R₂ is independently H, halogen, —OH, —OR₁, —OCR₁—CONR₁, OC(O)R₁, —OC(O)NH₂, OC(O)NHR₁, —OC(O)NR₁R₂, —NO₂, —NH₂, —NHRₜ, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SH, —SR₁, —S(O)H, —S(O)R₁, —SO₂R₁, —SO₂NH₂, —SO₂NHR₁, —SO₂NR₁R₂, CF₃, —CHF₂, —CH₂F, —OCF₃, —OCHF₂, —CN, —CO₂H, —CO₂R₁, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁; optionally substituted C₁₋₆ linear or branched alkyl group, an optionally substituted C₂₋₆ linear or branched alkenyl group, an optionally substituted C₂₋₆ alkynyl group; an optionally substituted C₃₋₇ cyclic alkyl group;
Z represents an optionally substituted aryl or an optionally substituted heteroaryl having up to 12 carbon atoms with one or more heteroatoms independently selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, halogen, —OH, —OR₁, —OCR₂ₐ, wherein R₂ₐ and R_{2b} are independently hydrogen, C₁-C₆ linear or branched alkyl, C₃₋₈ cycloalkyl, optionally substituted aryl, and an optionally substituted heteroaryl group having up to 12 carbon atoms and having one or more heteroatoms in its ring system which are each independently selected from O, N and S, aryl and hetro aryl may be further substituted with R₄;
R₄ represents C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ, —C(O)CH₂OH, —CH(CH₃)₂OH, C(O)R₄ₐR_{4b}OH, wherein R₄ₐ and R_{4b} are independently hydrogen, C₁-C₆ linear or branched alkyl, C₃₋₈ cycloalkyl, Halo, OH, NH₂, —CONH₂; and
n is 0 or an integer from 1-2.

In an embodiment, the present disclosure provides use of a compound of Formula (I), Formula (IA), Formula (II), Formula (IIA), Formula (IIB), Formula (III), Formula (IV), Formula (IVA), Formula (IVB) or Formula (V), or a pharmaceutically acceptable salt, polymorph, solvate or stereoisomer thereof, for manufacture of a medicament for treatment of a disorder characterized by excessive level of any or a combination of ALK5 and P38α MAP kinase.

In an embodiment, the present disclosure provides a pharmaceutical composition including a compound of Formula (I), Formula (IA), Formula (II), Formula (IIA), Formula (IIB), Formula (III), Formula (IV), Formula (IVA), Formula (IVB) or Formula (V), as defined above, or a pharmaceutically acceptable salt, polymorph, solvate or stereoisomer thereof, in combination with one or more pharmaceutically acceptable carrier(s), diluent(s) and/or excipient(s). A “pharmaceutically acceptable carrier, diluent, or excipient” is a medium generally accepted in the art for the delivery of biologically active agents to mammals, e.g., humans. Such carriers are generally formulated according to a number of factors well within the purview of those of ordinary skilled in the art to determine and account for. These include, without limitation: the type and nature of the active agent being formulated; the subject to which the agent-containing composition is to be administered; the intended route of administration of the composition; and the therapeutic indication being targeted. Pharmaceutically acceptable carriers and excipients include both aqueous and nonaqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985.

In an embodiment, the compounds of Formula (I), Formula (IA), Formula (II), Formula (IIA), Formula (IIB), Formula (IB), Formula (IV), Formula (IVA), Formula (IVB) or Formula (V), may be administered orally, topically, parenterally, by inhalation or spray or rectally in dosage unit formulations containing conventional non-toxic pharmaceutically acceptable carriers, adjuvants and vehicles. In an embodiment, the pharmaceutical compositions containing compounds of Formula (I), Formula (IA), Formula (II), Formula (IIA), Formula (IIB), Formula (III), Formula (IV), Formula (IVA), Formula (IVB) or Formula (V) may be in a form suitable for oral use, for example, as tablets, troches, lozenges, aqueous or oily suspensions, dispersible powders or granules, emulsion, hard or soft capsules, or syrups or elixirs. Dosage forms suitable for administration generally contain from about 1 mg to about 500 mg of active ingredient per unit. Appropriate coatings may be applied to increase palatability or delayed adsorption.

In an embodiment, the present disclosure relates to a method of treatment of cancer, wherein the method includes the step of administration to a patient, in need of such treatment, a compound of Formula (I), Formula (IA), Formula (II), Formula (IIA), Formula (IIB), Formula (III), Formula (IV), Formula (IVA), Formula (IVB) or Formula (V) as defined above, or a pharmaceutically acceptable salt, polymorph, solvate or stereoisomer thereof.

As used above, and throughout the description of the invention, the following terms, unless otherwise indicated, shall be understood to have the following meanings:

“Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-[beta]-hydroxynaphthoates, gentisates, isethionates, di-ptoluoyltartrates, methanesulphonates, ethanesulphonates, benzenesulphonates, ptoluenesulphonates, cyclohexylsulphamates and quinateslaurylsulphonate salts, and the like. See, for example S. M. Berge, et al., “Pharmaceutical Salts,” J. Pharm. Sci. 66, 1-19 (1977). Lists of suitable salts are found in Remington's Pharmaceutical Sciences, 17th ed., Mack Publishing. Company, Easton, Pa., 1985, p. 1418.

It will be understood that, as used herein, references to the compounds of Formula (I), Formula (IA), Formula (II), Formula (IIA), Formula (IIB), Formula (IB), Formula (IV), Formula (IVA), Formula (IVB) or Formula (V) are meant to also include the pharmaceutically acceptable salts thereof.

“Therapeutically effective amount” or “effective amount” means the amount of the compound of formula (I) of the present invention or pharmaceutical composition containing a compound of formula (I) of the present invention that will elicit the biological or medical response of or desired therapeutic effect on a tissue, system, animal or human that is being sought by the researcher, veterinarian, medical doctor or other clinician.

In an embodiment, the compound, as realized in accordance with the embodiments of the present disclosure, is any of:

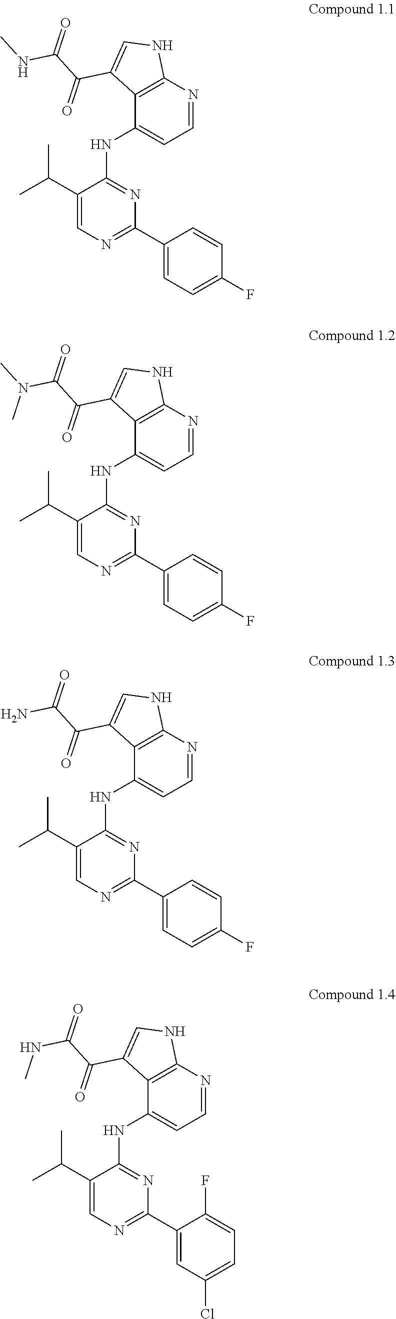 [SMILES: CNC(=O)C(=O)c1c[nH]c2nccc(Nc3nc(-c4ccc(F)cc4)ncc3C(C)C)c12 ; CC(C)c1cnc(-c2ccc(F)cc2)nc1Nc1ccnc2[nH]cc(C(=O)C(=O)N(C)C)c12 ; CC(C)c1cnc(-c2ccc(F)cc2)nc1Nc1ccnc2[nH]cc(C(=O)C(N)=O)c12 ; CNC(=O)C(=O)c1c[nH]c2nccc(Nc3nc(-c4cc(Cl)ccc4F)ncc3C(C)C)c12]

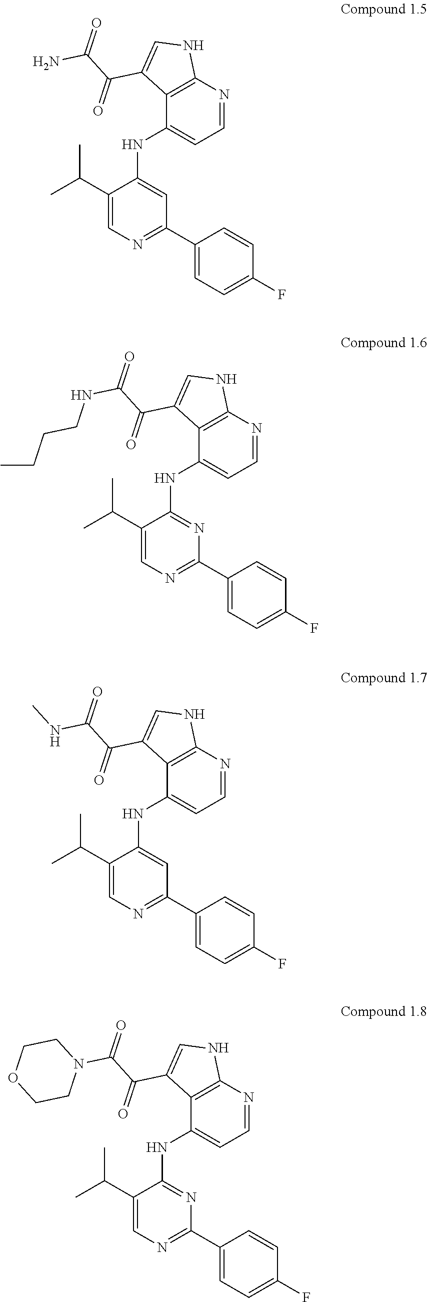 [SMILES: CC(C)c1cnc(-c2ccc(F)cc2)cc1Nc1ccnc2[nH]cc(C(=O)C(N)=O)c12 ; CCCCNC(=O)C(=O)c1c[nH]c2nccc(Nc3nc(-c4ccc(F)cc4)ncc3C(C)C)c12 ; CNC(=O)C(=O)c1c[nH]c2nccc(Nc3cc(-c4ccc(F)cc4)ncc3C(C)C)c12 ; CC(C)c1cnc(-c2ccc(F)cc2)nc1Nc1ccnc2[nH]cc(C(=O)C(=O)N3CCOCC3)c12]

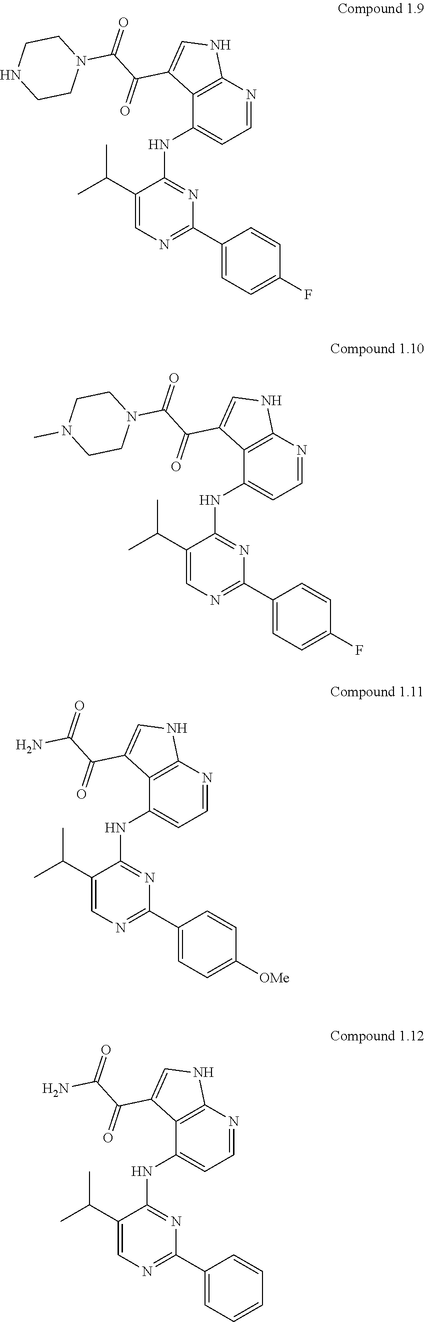 [SMILES: CC(C)c1cnc(-c2ccc(F)cc2)nc1Nc1ccnc2[nH]cc(C(=O)C(=O)N3CCNCC3)c12 ; CC(C)c1cnc(-c2ccc(F)cc2)nc1Nc1ccnc2[nH]cc(C(=O)C(=O)N3CCN(C)CC3)c12 ; COc1ccc(-c2ncc(C(C)C)c(Nc3ccnc4[nH]cc(C(=O)C(N)=O)c34)n2)cc1 ; CC(C)c1cnc(-c2ccccc2)nc1Nc1ccnc2[nH]cc(C(=O)C(N)=O)c12]

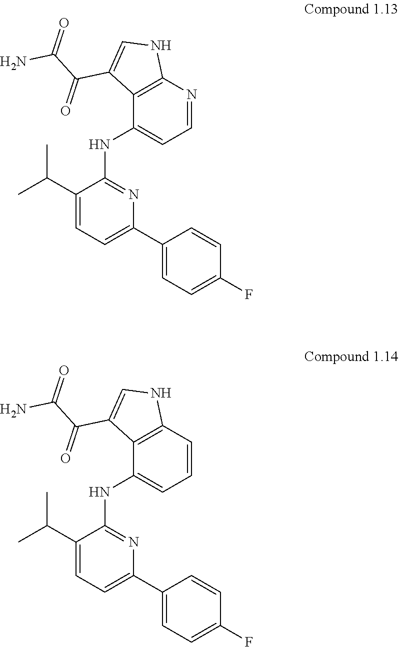 [SMILES: CC(C)c1ccc(-c2ccc(F)cc2)nc1Nc1ccnc2[nH]cc(C(=O)C(N)=O)c12 ; CC(C)c1ccc(-c2ccc(F)cc2)nc1Nc1cccc2[nH]cc(C(=O)C(N)=O)c12]

In an embodiment, the compound, as realized in accordance with the embodiments of the present disclosure, is any of:

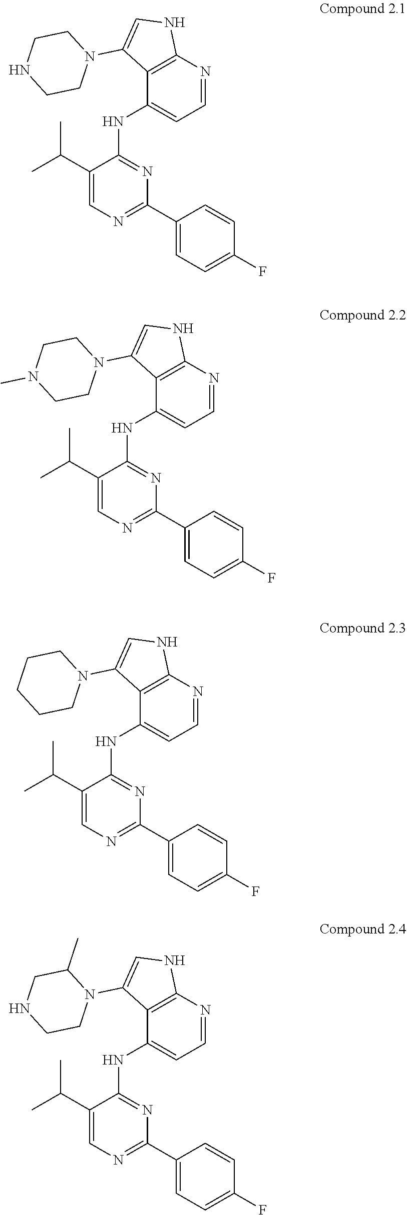 [SMILES: CC(C)c1cnc(-c2ccc(F)cc2)nc1Nc1ccnc2[nH]cc(N3CCNCC3)c12 ; CC(C)c1cnc(-c2ccc(F)cc2)nc1Nc1ccnc2[nH]cc(N3CCN(C)CC3)c12 ; CC(C)c1cnc(-c2ccc(F)cc2)nc1Nc1ccnc2[nH]cc(N3CCCCC3)c12 ; CC(C)c1cnc(-c2ccc(F)cc2)nc1Nc1ccnc2[nH]cc(N3CCNCC3C)c12]

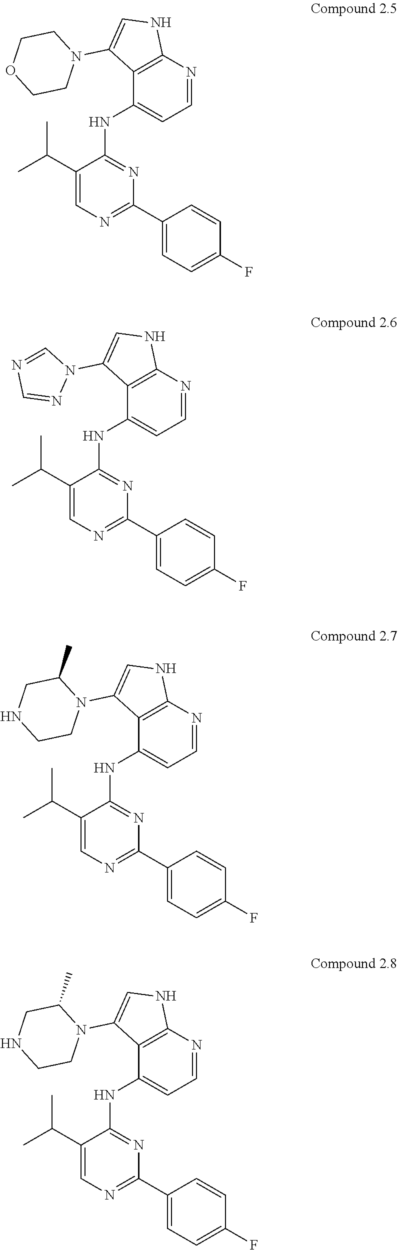 [SMILES: CC(C)c1cnc(-c2ccc(F)cc2)nc1Nc1ccnc2[nH]cc(N3CCOCC3)c12 ; CC(C)c1cnc(-c2ccc(F)cc2)nc1Nc1ccnc2[nH]cc(-n3cncn3)c12 ; CC(C)c1cnc(-c2ccc(F)cc2)nc1Nc1ccnc2[nH]cc(N3CCNC[C@H]3C)c12 ; CC(C)c1cnc(-c2ccc(F)cc2)nc1Nc1ccnc2[nH]cc(N3CCNC[C@@H]3C)c12]

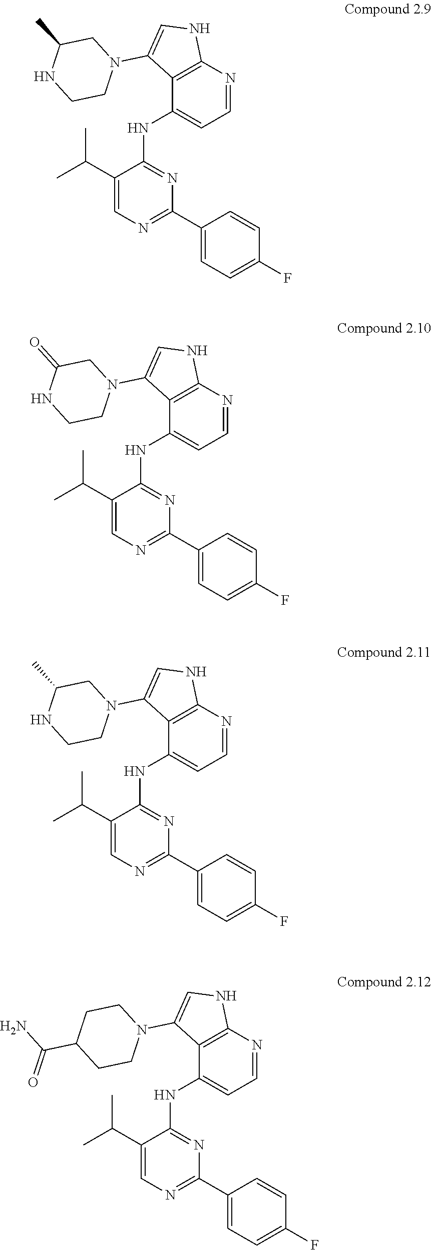 [SMILES: CC(C)c1cnc(-c2ccc(F)cc2)nc1Nc1ccnc2[nH]cc(N3CCN[C@@H](C)C3)c12 ; CC(C)c1cnc(-c2ccc(F)cc2)nc1Nc1ccnc2[nH]cc(N3CCNC(=O)C3)c12 ; CC(C)c1cnc(-c2ccc(F)cc2)nc1Nc1ccnc2[nH]cc(N3CCN[C@H](C)C3)c12 ; CC(C)c1cnc(-c2ccc(F)cc2)nc1Nc1ccnc2[nH]cc(N3CCC(C(N)=O)CC3)c12]

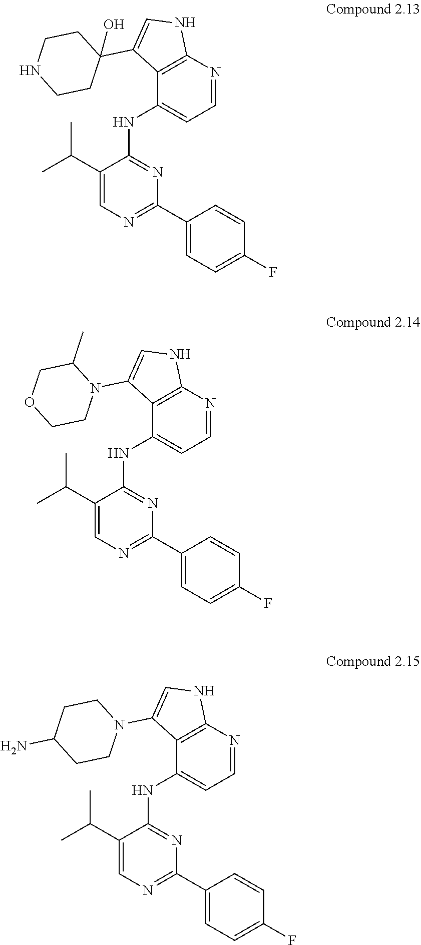 [SMILES: CC(C)c1cnc(-c2ccc(F)cc2)nc1Nc1ccnc2[nH]cc(C3(O)CCNCC3)c12 ; CC(C)c1cnc(-c2ccc(F)cc2)nc1Nc1ccnc2[nH]cc(N3CCOCC3C)c12 ; CC(C)c1cnc(-c2ccc(F)cc2)nc1Nc1ccnc2[nH]cc(N3CCC(N)CC3)c12]

In an embodiment, the compound, as realized in accordance with the embodiments of the present disclosure, is any of:

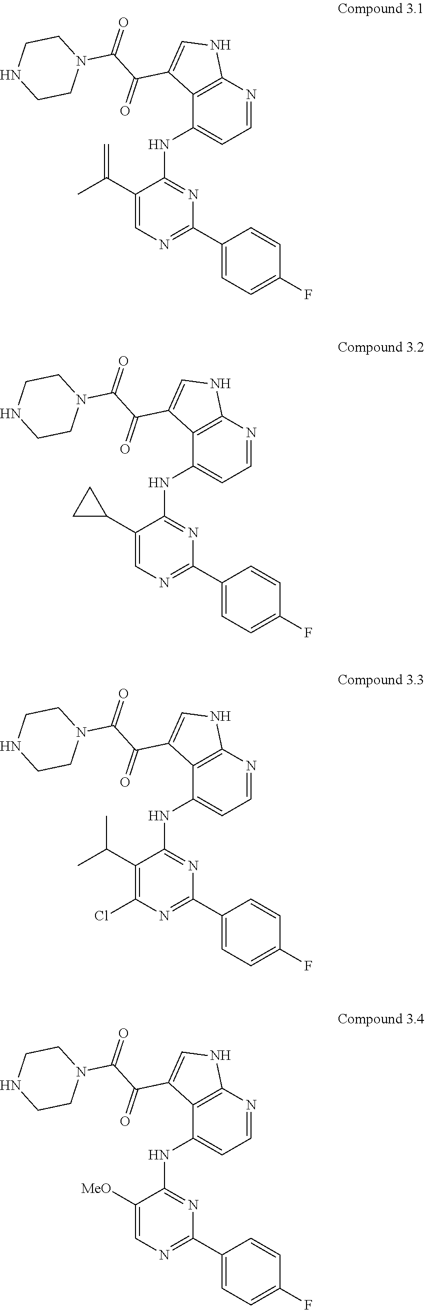 [SMILES: C=C(C)c1cnc(-c2ccc(F)cc2)nc1Nc1ccnc2[nH]cc(C(=O)C(=O)N3CCNCC3)c12 ; O=C(C(=O)N1CCNCC1)c1c[nH]c2nccc(Nc3nc(-c4ccc(F)cc4)ncc3C3CC3)c12 ; CC(C)c1c(Cl)nc(-c2ccc(F)cc2)nc1Nc1ccnc2[nH]cc(C(=O)C(=O)N3CCNCC3)c12 ; COc1cnc(-c2ccc(F)cc2)nc1Nc1ccnc2[nH]cc(C(=O)C(=O)N3CCNCC3)c12]

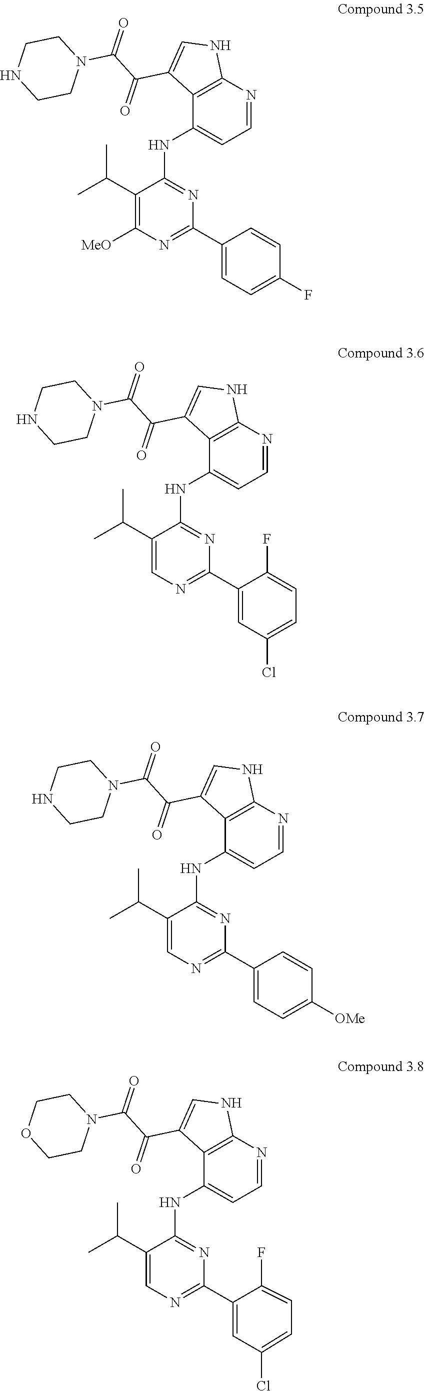 [SMILES: COc1nc(-c2ccc(F)cc2)nc(Nc2ccnc3[nH]cc(C(=O)C(=O)N4CCNCC4)c23)c1C(C)C ; CC(C)c1cnc(-c2cc(Cl)ccc2F)nc1Nc1ccnc2[nH]cc(C(=O)C(=O)N3CCNCC3)c12 ; COc1ccc(-c2ncc(C(C)C)c(Nc3ccnc4[nH]cc(C(=O)C(=O)N5CCNCC5)c34)n2)cc1 ; CC(C)c1cnc(-c2cc(Cl)ccc2F)nc1Nc1ccnc2[nH]cc(C(=O)C(=O)N3CCOCC3)c12]

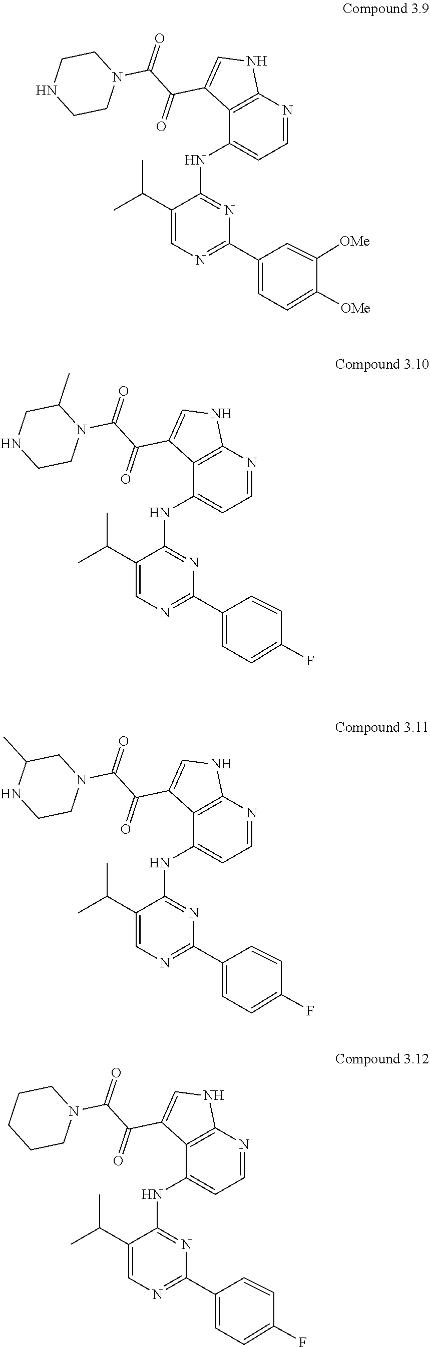 [SMILES: COc1ccc(-c2ncc(C(C)C)c(Nc3ccnc4[nH]cc(C(=O)C(=O)N5CCNCC5)c34)n2)cc1OC ; CC(C)c1cnc(-c2ccc(F)cc2)nc1Nc1ccnc2[nH]cc(C(=O)C(=O)N3CCNCC3C)c12 ; CC1CN(C(=O)C(=O)c2c[nH]c3nccc(Nc4nc(-c5ccc(F)cc5)ncc4C(C)C)c23)CCN1 ; CC(C)c1cnc(-c2ccc(F)cc2)nc1Nc1ccnc2[nH]cc(C(=O)C(=O)N3CCCCC3)c12]

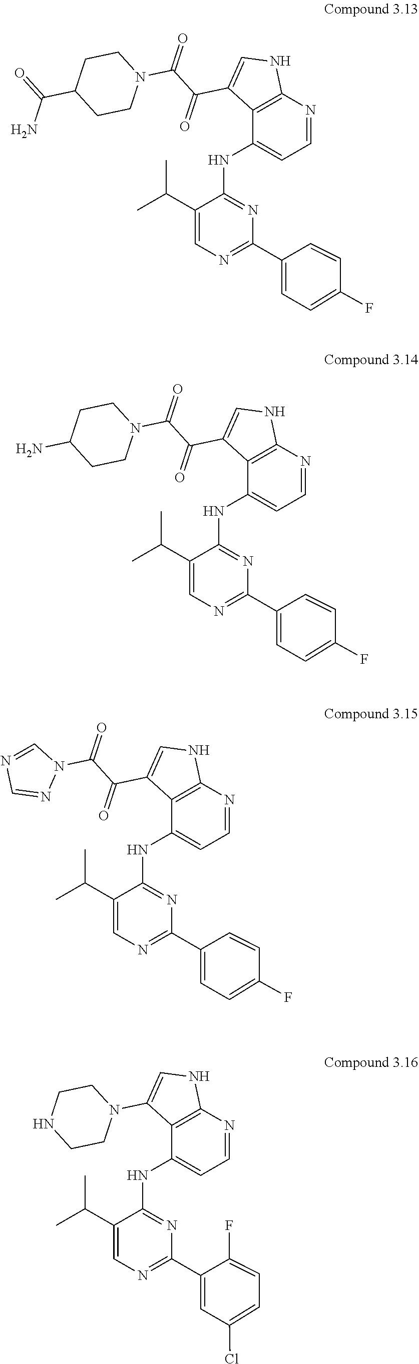 [SMILES: CC(C)c1cnc(-c2ccc(F)cc2)nc1Nc1ccnc2[nH]cc(C(=O)C(=O)N3CCC(C(N)=O)CC3)c12 ; CC(C)c1cnc(-c2ccc(F)cc2)nc1Nc1ccnc2[nH]cc(C(=O)C(=O)N3CCC(N)CC3)c12 ; CC(C)c1cnc(-c2ccc(F)cc2)nc1Nc1ccnc2[nH]cc(C(=O)C(=O)n3cncn3)c12 ; CC(C)c1cnc(-c2cc(Cl)ccc2F)nc1Nc1ccnc2[nH]cc(N3CCNCC3)c12]

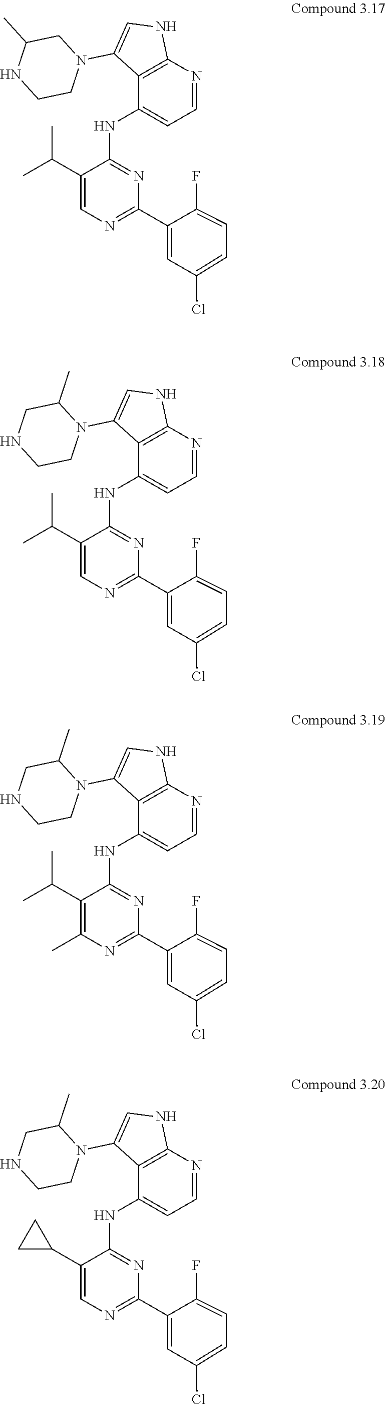 [SMILES: CC1CN(c2c[nH]c3nccc(Nc4nc(-c5cc(Cl)ccc5F)ncc4C(C)C)c23)CCN1 ; CC(C)c1cnc(-c2cc(Cl)ccc2F)nc1Nc1ccnc2[nH]cc(N3CCNCC3C)c12 ; Cc1nc(-c2cc(Cl)ccc2F)nc(Nc2ccnc3[nH]cc(N4CCNCC4C)c23)c1C(C)C ; CC1CNCCN1c1c[nH]c2nccc(Nc3nc(-c4cc(Cl)ccc4F)ncc3C3CC3)c12]

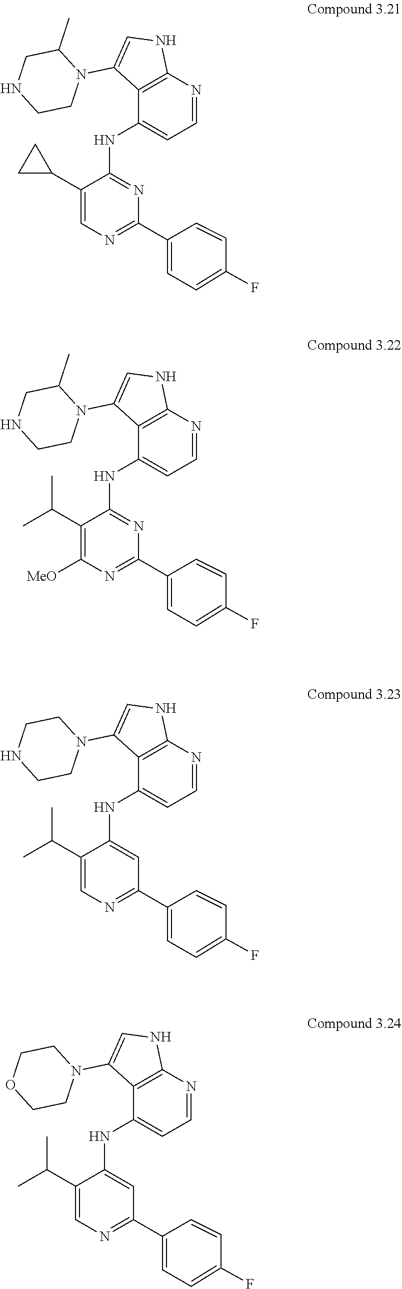 [SMILES: CC1CNCCN1c1c[nH]c2nccc(Nc3nc(-c4ccc(F)cc4)ncc3C3CC3)c12 ; COc1nc(-c2ccc(F)cc2)nc(Nc2ccnc3[nH]cc(N4CCNCC4C)c23)c1C(C)C ; CC(C)c1cnc(-c2ccc(F)cc2)cc1Nc1ccnc2[nH]cc(N3CCNCC3)c12 ; CC(C)c1cnc(-c2ccc(F)cc2)cc1Nc1ccnc2[nH]cc(N3CCOCC3)c12]

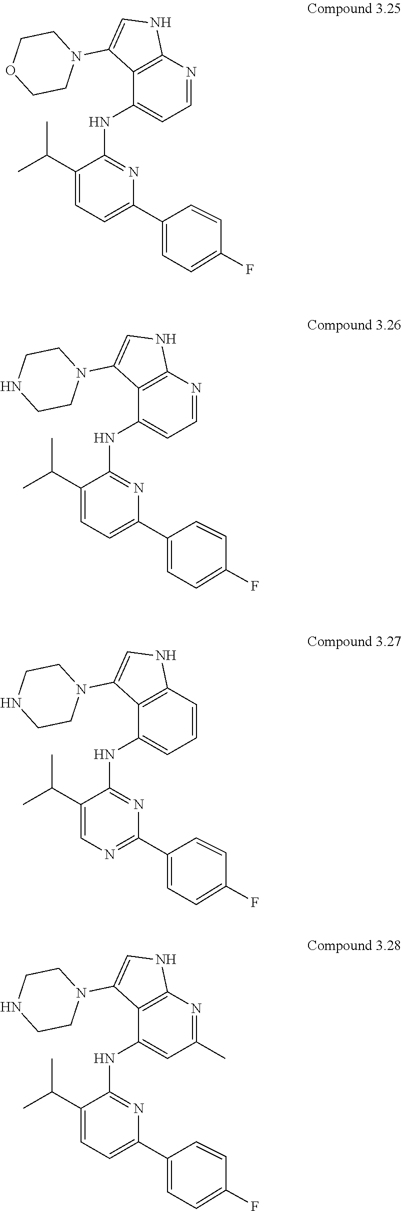 [SMILES: CC(C)c1ccc(-c2ccc(F)cc2)nc1Nc1ccnc2[nH]cc(N3CCOCC3)c12 ; CC(C)c1ccc(-c2ccc(F)cc2)nc1Nc1ccnc2[nH]cc(N3CCNCC3)c12 ; CC(C)c1cnc(-c2ccc(F)cc2)nc1Nc1cccc2[nH]cc(N3CCNCC3)c12 ; Cc1cc(Nc2nc(-c3ccc(F)cc3)ccc2C(C)C)c2c(N3CCNCC3)c[nH]c2n1]

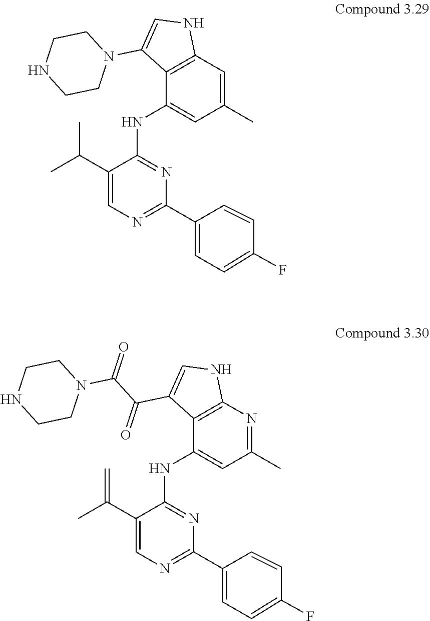 [SMILES: Cc1cc(Nc2nc(-c3ccc(F)cc3)ncc2C(C)C)c2c(N3CCNCC3)c[nH]c2c1 ; C=C(C)c1cnc(-c2ccc(F)cc2)nc1Nc1cc(C)nc2[nH]cc(C(=O)C(=O)N3CCNCC3)c12]

In an embodiment, the compound, as realized in accordance with the embodiments of the present disclosure, is any of:
N-(2-(4-fluorophenyl)-5-isopropylpyrimidin-4-yl)-3-(piperazin-1-yl)-1H-pyrrolo [2,3-b] pyridin-4-amine.
N-(2-(4-fluorophenyl)-5-isopropylpyrimidin-4-yl)-3-(4-methylpiperazin-1-yl)-1H-pyrrolo [2,3-b] pyridin-4-amine.
N-(2-(4-fluorophenyl)-5-isopropylpyrimidin-4-yl)-3-(piperidin-1-yl)-1H-pyrrolo [2, 3-b] pyridin-4-amine.
N-(2-(4-fluorophenyl)-5-isopropylpyrimidin-4-yl)-3-(2-methylpiperazin-1-yl)-1H-pyrrolo[2,3-b]pyridin-4-amine.
N-(2-(4-fluorophenyl)-5-isopropylpyrimidin-4-yl)-3-morpholino-1H-pyrrolo [2, 3-b]pyridin-4-amine.
N-(2-(4-fluorophenyl)-5-isopropylpyrimidin-4-yl)-3-(1H-1, 2, 4-triazol-1-yl)-1H-pyrrolo [2,3-b] pyridin-4-amine.
N-(2-(4-fluorophenyl)-5-isopropylpyrimidin-4-yl)-3-(2-methylpiperazin-1-yl)-1H-pyrrolo [2,3-b] pyridin-4-amine.
N-(2-(4-fluorophenyl)-5-isopropylpyrimidin-4-yl)-3-(2-methylpiperazin-1-yl)-1H-pyrrolo [2,3-b] pyridin-4-amine.
(R)—N-(2-(4-fluorophenyl)-5-isopropylpyrimidin-4-yl)-3-(3-methylpiperazin-1-yl)-1H-pyrrolo[2,3-b]pyridin-4-amine.
4-(4-(2-(4-fluorophenyl)-5-isopropylpyrimidin-4-ylamino)-1H-pyrrolo [2, 3-b] pyridin-3-yl) piperazin-2-one formate.
(S)—N-(2-(4-fluorophenyl)-5-isopropylpyrimidin-4-yl)-3-(3-methylpiperazin-1-yl)-1H-pyrrolo[2,3-b]pyridin-4-amine.
1-(4-((2-(4-fluorophenyl)-5-isopropylpyrimidin-4-yl)amino)-1H-pyrrolo[2,3-b]pyridin-3-yl)piperidine-4-carboxamide formic acid.
4-(4-((2-(4-fluorophenyl)-5-isopropylpyrimidin-4-yl)amino)-1H-pyrrolo[2,3-b]pyridin-3-yl)piperidin-4-ol.
N-(2-(4-fluorophenyl)-5-isopropylpyrimidin-4-yl)-3-(3-methylmorpholino)-1H-pyrrolo
[2,3-b] pyridin-4-amine.
3-(4-aminopiperidin-1-yl)-N-(2-(4-fluorophenyl)-5-isopropylpyrimidin-4-yl)-1H-pyrrolo[2,3-b]pyridin-4-amine.

The compounds of the present disclosure may be prepared by a number of processes as known to or appreciated by a person skilled in the pertinent art, and more specifically, as illustrated in the Examples section provided hereinbelow. In the following process description, the symbols Ar₁, R₁, A, B, R₄, P, Q, R, T, X, Y, Z, m and n, when used in the Formula, are to be understood to represent those groups described above in relation to Formula (I) unless otherwise indicated.

Table 1 below illustrates the compounds realized in accordance with embodiments of the present disclosure.

| Compound | |
|---|---|
| No | Structure |
| 1 | 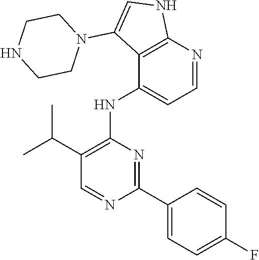 [SMILES: CC(C)c1cnc(-c2ccc(F)cc2)nc1Nc1ccnc2[nH]cc(N3CCNCC3)c12] |
| 2 | 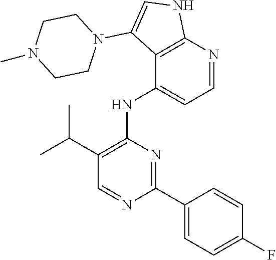 [SMILES: CC(C)c1cnc(-c2ccc(F)cc2)nc1Nc1ccnc2[nH]cc(N3CCN(C)CC3)c12] |
| 3 | 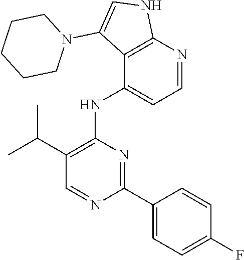 [SMILES: CC(C)c1cnc(-c2ccc(F)cc2)nc1Nc1ccnc2[nH]cc(N3CCCCC3)c12] |
| 4 | 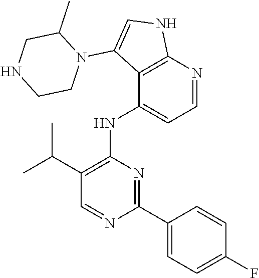 [SMILES: CC(C)c1cnc(-c2ccc(F)cc2)nc1Nc1ccnc2[nH]cc(N3CCNCC3C)c12] |
| 5 | 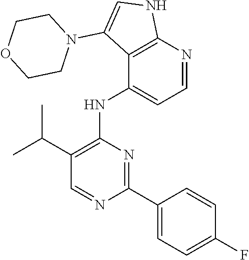 [SMILES: CC(C)c1cnc(-c2ccc(F)cc2)nc1Nc1ccnc2[nH]cc(N3CCOCC3)c12] |
| 6 | 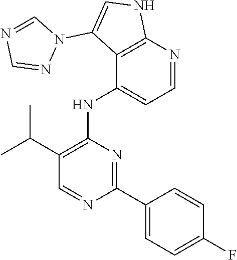 [SMILES: CC(C)c1cnc(-c2ccc(F)cc2)nc1Nc1ccnc2[nH]cc(-n3cncn3)c12] |
| 7 | 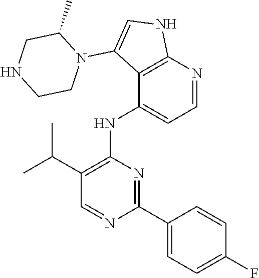 [SMILES: CC(C)c1cnc(-c2ccc(F)cc2)nc1Nc1ccnc2[nH]cc(N3CCNC[C@@H]3C)c12] |
| 8 | 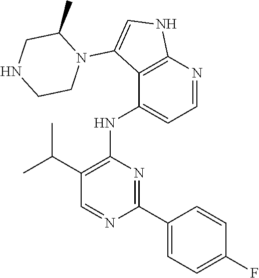 [SMILES: CC(C)c1cnc(-c2ccc(F)cc2)nc1Nc1ccnc2[nH]cc(N3CCNC[C@H]3C)c12] |
| 9 | 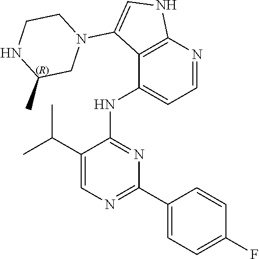 |
| 10 | 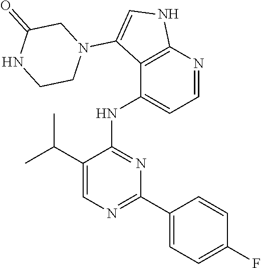 [SMILES: CC(C)c1cnc(-c2ccc(F)cc2)nc1Nc1ccnc2[nH]cc(N3CCNC(=O)C3)c12] |
| 11 | 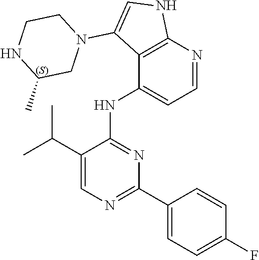 |
| 12 | 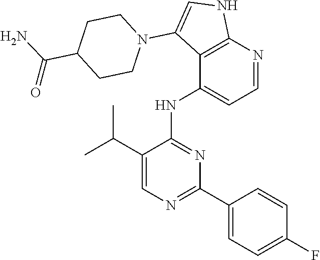 [SMILES: CC(C)c1cnc(-c2ccc(F)cc2)nc1Nc1ccnc2[nH]cc(N3CCC(C(N)=O)CC3)c12] |
| 13 | 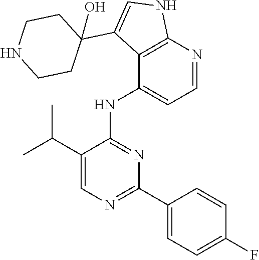 [SMILES: CC(C)c1cnc(-c2ccc(F)cc2)nc1Nc1ccnc2[nH]cc(C3(O)CCNCC3)c12] |
| 14 | 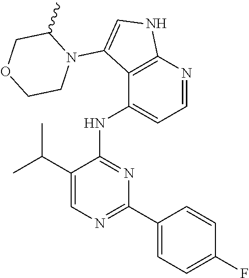 [SMILES: CC(C)c1cnc(-c2ccc(F)cc2)nc1Nc1ccnc2[nH]cc(N3CCOCC3C)c12] |
| 15 | 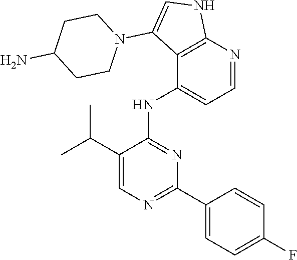 [SMILES: CC(C)c1cnc(-c2ccc(F)cc2)nc1Nc1ccnc2[nH]cc(N3CCC(N)CC3)c12] |
| 16 | 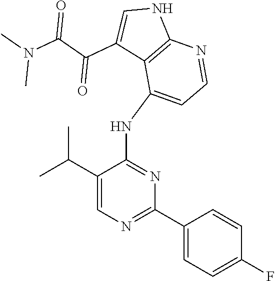 [SMILES: CC(C)c1cnc(-c2ccc(F)cc2)nc1Nc1ccnc2[nH]cc(C(=O)C(=O)N(C)C)c12] |
| 17 | 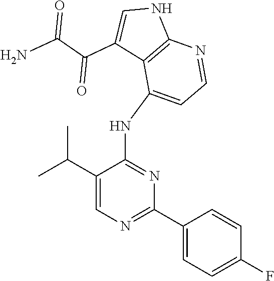 [SMILES: CC(C)c1cnc(-c2ccc(F)cc2)nc1Nc1ccnc2[nH]cc(C(=O)C(N)=O)c12] |
| 18 | 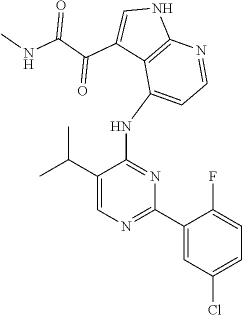 [SMILES: CNC(=O)C(=O)c1c[nH]c2nccc(Nc3nc(-c4cc(Cl)ccc4F)ncc3C(C)C)c12] |
| 19 | 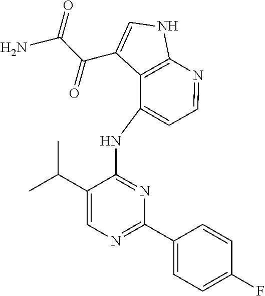 [SMILES: CC(C)c1cnc(-c2ccc(F)cc2)nc1Nc1ccnc2[nH]cc(C(=O)C(N)=O)c12] |
| 20 | 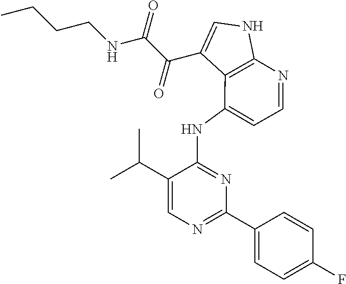 [SMILES: CCCCNC(=O)C(=O)c1c[nH]c2nccc(Nc3nc(-c4ccc(F)cc4)ncc3C(C)C)c12] |
| 21 | 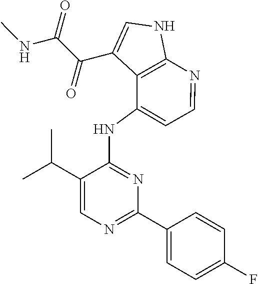 [SMILES: CNC(=O)C(=O)c1c[nH]c2nccc(Nc3nc(-c4ccc(F)cc4)ncc3C(C)C)c12] |
| 22 | 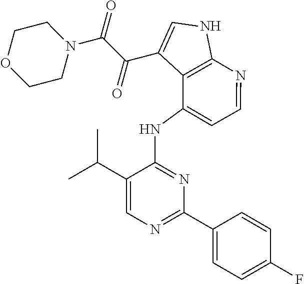 [SMILES: CC(C)c1cnc(-c2ccc(F)cc2)nc1Nc1ccnc2[nH]cc(C(=O)C(=O)N3CCOCC3)c12] |
| 23 | 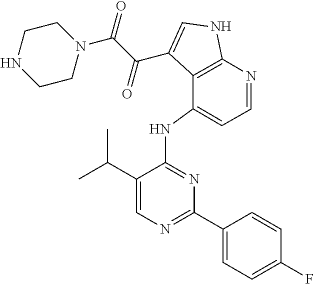 [SMILES: CC(C)c1cnc(-c2ccc(F)cc2)nc1Nc1ccnc2[nH]cc(C(=O)C(=O)N3CCNCC3)c12] |
| 24 | 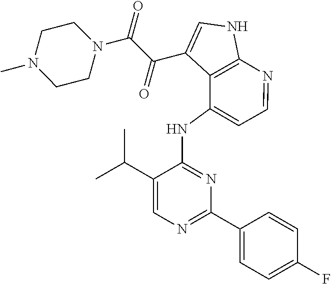 [SMILES: CC(C)c1cnc(-c2ccc(F)cc2)nc1Nc1ccnc2[nH]cc(C(=O)C(=O)N3CCN(C)CC3)c12] |
| 25 | 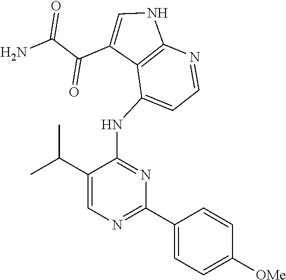 [SMILES: COc1ccc(-c2ncc(C(C)C)c(Nc3ccnc4[nH]cc(C(=O)C(N)=O)c34)n2)cc1] |
| 26 | 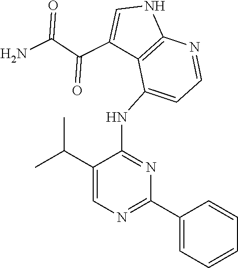 [SMILES: CC(C)c1cnc(-c2ccccc2)nc1Nc1ccnc2[nH]cc(C(=O)C(N)=O)c12] |
| 27 | 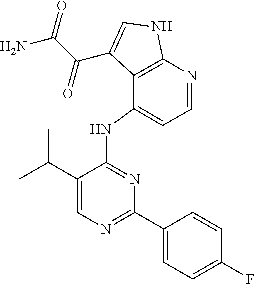 [SMILES: CC(C)c1cnc(-c2ccc(F)cc2)nc1Nc1ccnc2[nH]cc(C(=O)C(N)=O)c12] |
| 28 | 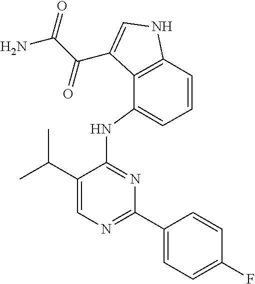 [SMILES: CC(C)c1cnc(-c2ccc(F)cc2)nc1Nc1cccc2[nH]cc(C(=O)C(N)=O)c12] |
| 29 | 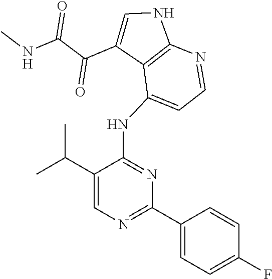 [SMILES: CNC(=O)C(=O)c1c[nH]c2nccc(Nc3nc(-c4ccc(F)cc4)ncc3C(C)C)c12] |
| 30 | 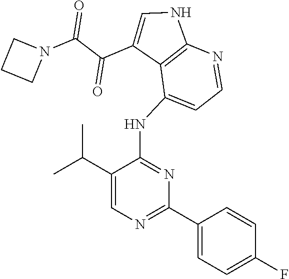 [SMILES: CC(C)c1cnc(-c2ccc(F)cc2)nc1Nc1ccnc2[nH]cc(C(=O)C(=O)N3CCC3)c12] |
| 31 | 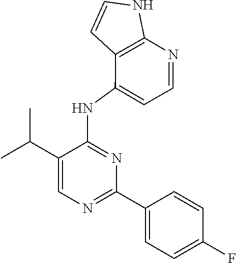 [SMILES: CC(C)c1cnc(-c2ccc(F)cc2)nc1Nc1ccnc2[nH]ccc12] |
| 32 | 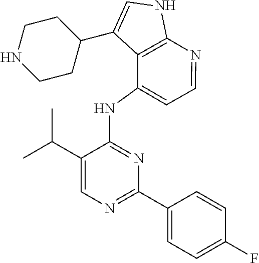 [SMILES: CC(C)c1cnc(-c2ccc(F)cc2)nc1Nc1ccnc2[nH]cc(C3CCNCC3)c12] |
| 33 | 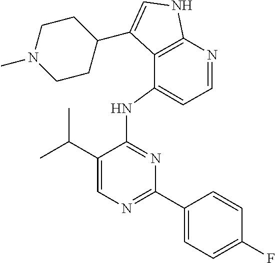 [SMILES: CC(C)c1cnc(-c2ccc(F)cc2)nc1Nc1ccnc2[nH]cc(C3CCN(C)CC3)c12] |
| 34 | 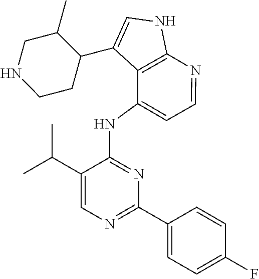 [SMILES: CC(C)c1cnc(-c2ccc(F)cc2)nc1Nc1ccnc2[nH]cc(C3CCNCC3C)c12] |
| 35 | 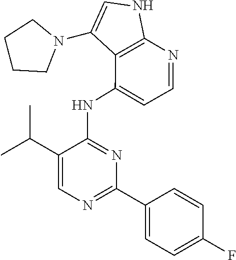 [SMILES: CC(C)c1cnc(-c2ccc(F)cc2)nc1Nc1ccnc2[nH]cc(N3CCCC3)c12] |
| 36 | 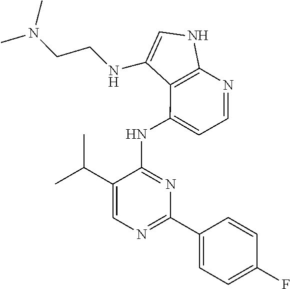 [SMILES: CC(C)c1cnc(-c2ccc(F)cc2)nc1Nc1ccnc2[nH]cc(NCCN(C)C)c12] |
| 37 | 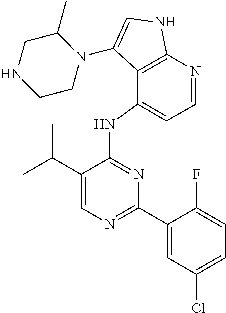 [SMILES: CC(C)c1cnc(-c2cc(Cl)ccc2F)nc1Nc1ccnc2[nH]cc(N3CCNCC3C)c12] |
| 38 | 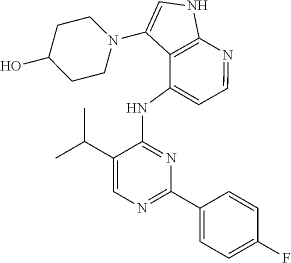 [SMILES: CC(C)c1cnc(-c2ccc(F)cc2)nc1Nc1ccnc2[nH]cc(N3CCC(O)CC3)c12] |
| 39 | 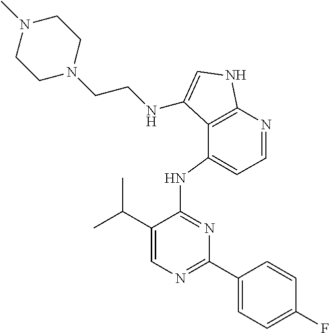 [SMILES: CC(C)c1cnc(-c2ccc(F)cc2)nc1Nc1ccnc2[nH]cc(NCCN3CCN(C)CC3)c12] |
| 40 | 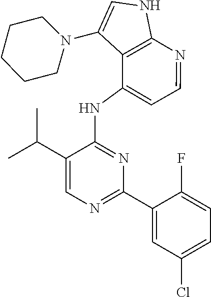 [SMILES: CC(C)c1cnc(-c2cc(Cl)ccc2F)nc1Nc1ccnc2[nH]cc(N3CCCCC3)c12] |
| 41 | 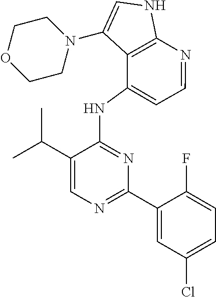 [SMILES: CC(C)c1cnc(-c2cc(Cl)ccc2F)nc1Nc1ccnc2[nH]cc(N3CCOCC3)c12] |
| 42 | 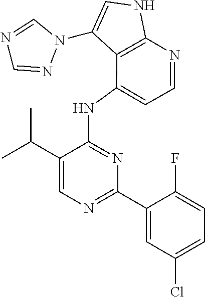 [SMILES: CC(C)c1cnc(-c2cc(Cl)ccc2F)nc1Nc1ccnc2[nH]cc(-n3cncn3)c12] |
| 43 | 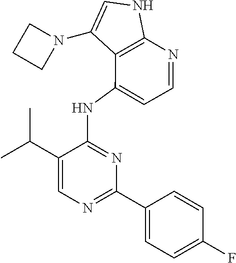 [SMILES: CC(C)c1cnc(-c2ccc(F)cc2)nc1Nc1ccnc2[nH]cc(N3CCC3)c12] |
| 44 | 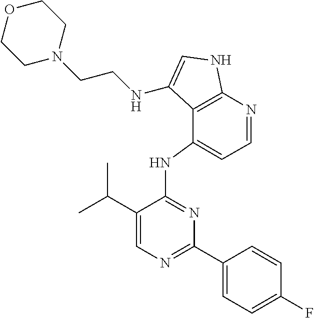 [SMILES: CC(C)c1cnc(-c2ccc(F)cc2)nc1Nc1ccnc2[nH]cc(NCCN3CCOCC3)c12] |
| 45 | 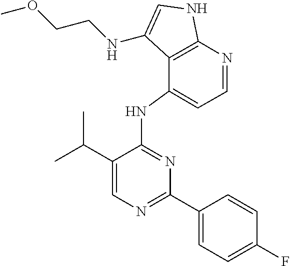 [SMILES: COCCNc1c[nH]c2nccc(Nc3nc(-c4ccc(F)cc4)ncc3C(C)C)c12] |
| 46 | 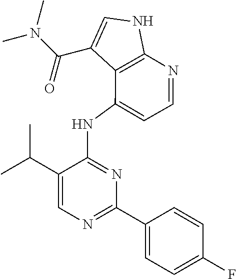 [SMILES: CC(C)c1cnc(-c2ccc(F)cc2)nc1Nc1ccnc2[nH]cc(C(=O)N(C)C)c12] |
| 47 | 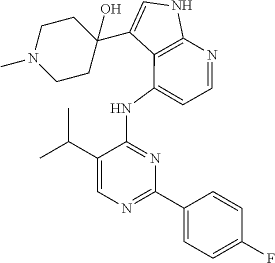 [SMILES: CC(C)c1cnc(-c2ccc(F)cc2)nc1Nc1ccnc2[nH]cc(C3(O)CCN(C)CC3)c12] |
| 48 | 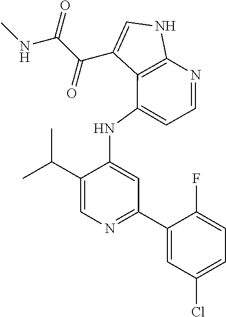 [SMILES: CNC(=O)C(=O)c1c[nH]c2nccc(Nc3cc(-c4cc(Cl)ccc4F)ncc3C(C)C)c12] |
| 49 | 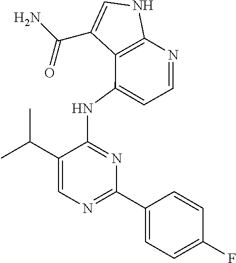 [SMILES: CC(C)c1cnc(-c2ccc(F)cc2)nc1Nc1ccnc2[nH]cc(C(N)=O)c12] |
| 50 | 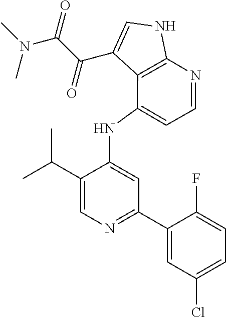 [SMILES: CC(C)c1cnc(-c2cc(Cl)ccc2F)cc1Nc1ccnc2[nH]cc(C(=O)C(=O)N(C)C)c12] |
| 51 | 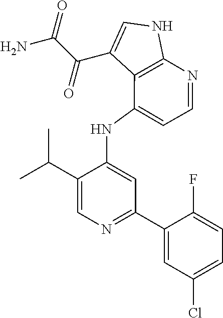 [SMILES: CC(C)c1cnc(-c2cc(Cl)ccc2F)cc1Nc1ccnc2[nH]cc(C(=O)C(N)=O)c12] |
| 52 | 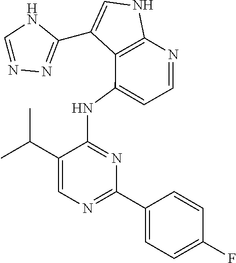 [SMILES: CC(C)c1cnc(-c2ccc(F)cc2)nc1Nc1ccnc2[nH]cc(-c3nnc[nH]3)c12] |
| 53 | 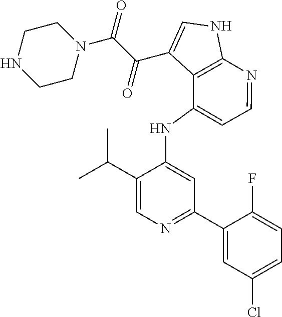 [SMILES: CC(C)c1cnc(-c2cc(Cl)ccc2F)cc1Nc1ccnc2[nH]cc(C(=O)C(=O)N3CCNCC3)c12] |
| 54 | 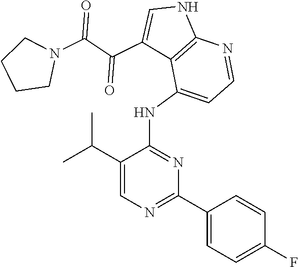 [SMILES: CC(C)c1cnc(-c2ccc(F)cc2)nc1Nc1ccnc2[nH]cc(C(=O)C(=O)N3CCCC3)c12] |
| 55 | 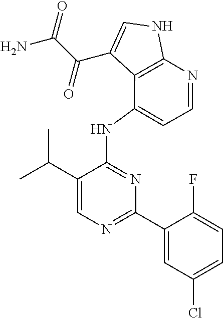 [SMILES: CC(C)c1cnc(-c2cc(Cl)ccc2F)nc1Nc1ccnc2[nH]cc(C(=O)C(N)=O)c12] |

### Example 1: Preparation of N-(2-(4-fluorophenyl)-5-isopropylpyrimidin-4-yl)-3-(piperazin-1-yl)-1H-pyrrolo [2, 3-b] pyridin-4-amine hydrochloride (Compound No. 1)

N-(2-(4-fluorophenyl)-5-isopropylpyrimidin-4-yl)-3-(piperazin-1-yl)-1H-pyrrolo [2, 3-b] pyridin-4-amine hydrochloride (Compound No. 1) was prepared according to steps illustrated in Scheme 1:

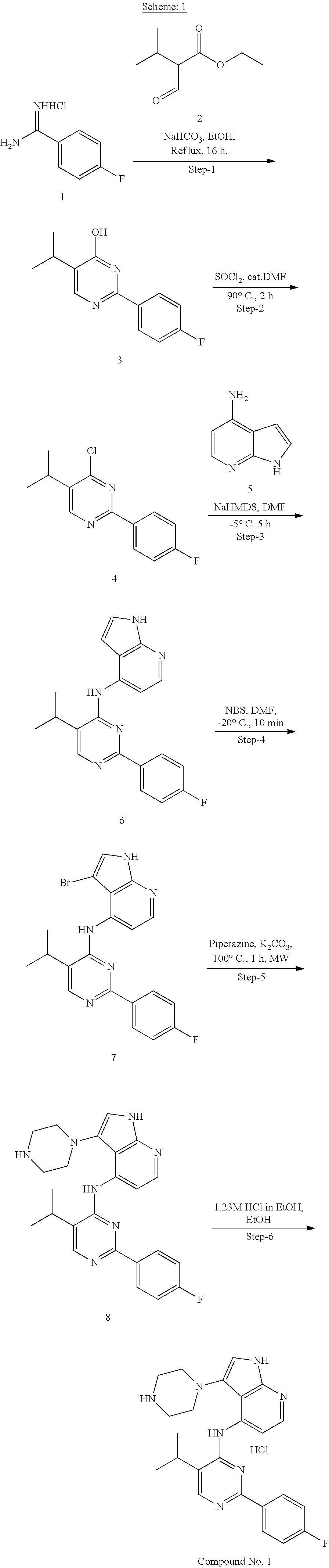

### Step 1: Synthesis of 2-(4-fluorophenyl)-5-isopropylpyrimidin-4-ol

To the solution of 4-fluorobenzimidamide hydrochloride (1.0 g, 5.7 mmol, 1.0 eq.) and ethyl 2-formyl-3-methylbutanoate (1.17 g, 7.48 mmol, 1.3 eq.) in ethanol (20 ml) was added NaHCO₃ (1.44 g, 17.06 mmol, 3.0 eq). Reaction mass was refluxed at 85° C. for 16 hours. The progress of reaction was monitored by TLC and LCMS. Upon completion, the ethanol was removed under reduced pressure to get crude product. Crude product was triturated with n-pentane to give 1.0 g of 2-(4-fluorophenyl)-5-isopropylpyrimidin-4-ol. LCMS (M+1): 234.1 ¹H-NMR: (400 MHz, DMSO-d₆): δ 8.08-8.21 (m, 2H), 7.89 (s, 1H), 7.34 (t, J=8.77 Hz, 2H), 2.84-2.94 (m, 1H), 1.18 (d, J=7.02 Hz, 6H).

### Step 2: Synthesis of 4-chloro-2-(4-fluorophenyl)-5-isopropylpyrimidine

To the suspension of 2-(4-fluorophenyl)-5-isopropylpyrimidin-4-ol (1.0 g, 4.3 mmol) in 5 ml thionyl chloride, taken in 25 ml sealed tube, was added 2-3 drops of DMF. Reaction mass was heated at 90° C. for 2 hours. Progress of reaction was monitored by TLC. Upon completion of reaction, thionyl chloride was removed under reduced pressure. Remaining thionyl chloride was dissolved in ethyl acetate 80 ml, and then organic layer was washed with 10% NaHCO₃ twice to remove remaining thionyl chloride followed by water and brine solution. Dried over anhydrous Na₂SO₄, and solvent was evaporated under reduced pressure at low temperature to obtain 0.8 g of 4-chloro-2-(4-fluorophenyl)-5-isopropylpyrimidine as yellow solid. LCMS (M+1): 251.1 ¹H-NMR (400 MHz, CDCl₃): δ 8.42 (dd, J=6.14, 8.33 Hz, 2H), 7.15 (t, J=8.55 Hz, 2H), 3.30-3.33 (m, 1H), 1.35 (d, J=7.02 Hz, 6H).

### Step 3: Synthesis of N-(2-(4-fluorophenyl)-5-isopropylpyrimidin-4-yl)-1H-pyrrolo [2,3-b] pyridin-4-amine

To the solution of compound 4-amino 7-azaindole (0.5 g, 37.5 mmol, 1.0 eq) in 50 ml DMF, cooled at −5° C., were added 4-chloro-2-(4-fluorophenyl)-5-isopropylpyrimidine (0.93 g, 37.5 mmol, 1.0 eq), followed by drop wise addition of solution of NaHMDS (35.5 ml, 22.5 mmol, 35% in THF) in two lots, under N₂ atmosphere. Reaction mass was continued at same temperature for 5 hours. After completion of reaction (TLC monitoring), reaction mass was poured in 200 g of ice cooled water. Resulting solid was filtered out to get 0.7 g of crude product. Crude product was purified by silica gel column chromatography (100-200) mesh silica with 0-3% MeOH:DCM to get 0.6 g of N-(2-(4-fluorophenyl)-5-isopropylpyrimidin-4-yl)-1H-pyrrolo[2,3-b]pyridin-4-amine. LCMS (M+1): 348.2

¹H-NMR: (400 MHz, DMSO-d₆): δ 11.57 (br. s., 1H), 8.78 (s, 1H), 8.48 (s, 1H), 8.13-8.28 (m, 3H), 7.50 (d, J=5.26 Hz, 1H), 7.32-7.37 (m, 1H), 7.25 (t, J=8.99 Hz, 2H), 6.43 (dd, J=1.97, 3.29 Hz, 1H), 3.41-3.52 (m, 1H), 1.33 (d, J=6.58 Hz, 6H).

### Step 4: Synthesis of 3-bromo-N-(2-(4-fluorophenyl)-5-isopropylpyrimidin-4-yl)-1H-pyrrolo[2,3-b]pyridin-4-amine

To the solution of N-(2-(4-fluorophenyl)-5-isopropylpyrimidin-4-yl)-1H-pyrrolo[2,3-b]pyridin-4-amine (1.0 g, 2.80 mmol, 1.0 eq) in 40 ml DMF, cooled at −20° C., was added NBS (0.614 g, 3.40 mmol, 1.2 eq.) portion wise under N₂ atmosphere. Reaction mass was stirred at same temperature for 10 minutes. After completion of reaction, reaction mass was poured in ice cooled water. Product was extracted in ethyl acetate. Organic layer was washed by brine solution (2×100 ml) then dried over anhydrous Na₂SO₄ and solvent was evaporated under reduced pressure at low temperature to obtain 0.550 g of 3-bromo-N-(2-(4-fluorophenyl)-5-isopropylpyrimidin-4-yl)-1H-pyrrolo[2,3-b]pyridin-4-amine. LCMS (M+H)=426.1, (M+2)=428.1 ¹H NMR (400 MHz, DMSO-d₆): δ 12.10 (br. s., 1H), 8.77 (s, 1H), 8.51 (s, 1H), 8.28 (d, J=5.26 Hz, 1H), 8.20-8.26 (m, 2H), 7.96 (d, J=5.26 Hz, 1H), 7.63 (br. s., 1H), 7.28 (t, J=8.55 Hz, 2H), 3.31-3.26 (m, 1H), 1.37 (d, J=6.58 Hz, 6H)

### Step 5: Synthesis of N-(2-(4-fluorophenyl)-5-isopropylpyrimidin-4-yl)-3-(piperazin-1-yl)-1H-pyrrolo[2,3-b] pyridin-4-amine

To a solution of 3-bromo-N-(2-(4-fluorophenyl)-5-isopropylpyrimidin-4-yl)-1H-pyrrolo[2,3-b]pyridin-4-amine (0.100 g, 0.234 mmol, 1.0 eq) in 3 ml of DMF, were added piperazine (0.100 g, 0.110 mmol, 5.0 eq) and K₂CO₃ (0.60 g, 0.47 mmol, 2.0 eq). Reaction mass was heated at 100° C. for 1 hour under microwave. After completion of reaction (LCMS monitoring), reaction mass was filtered to remove K₂CO₃ and filtrate was concentrated on high vacuum pump and crude reaction mixture was purified by PREP-HPLC to give 0.050 g of N-(2-(4-fluorophenyl)-5-isopropylpyrimidin-4-yl)-3-(piperazin-1-yl)-1H-pyrrolo[2,3-h]pyridin-4-amine as pale yellow solid. Compound was characterized by ¹H-NMR. LCMS (M+1): 432.2 ¹H NMR (400 MHz, DMSO-d₆): δ 11.42 (br. s., 1H), 9.77 (s, 1H), 8.58 (s, 1H), 8.42 (dd, J=5.70, 8.77 Hz, 2H), 8.31 (d, J=5.26 Hz, 1H), 8.18 (d, J=5.70 Hz, 2H), 7.37-7.42 (m, 3H), 7.36 (s, 1H), 3.41 (m, 1H), 2.97 (br. s., 9H), 1.38 (d, J=6.58 Hz, 6H)

### Step 6: Synthesis of N-(2-(4-fluorophenyl)-5-isopropylpyrimidin-4-yl)-3-(piperazin-1-yl)-1H-pyrrolo [2,3-b] pyridin-4-amine hydrochloride

To the clear solution of N-(2-(4-fluorophenyl)-5-isopropylpyrimidin-4-yl)-3-(piperazin-1-yl)-1H-pyrrolo[2,3-b]pyridin-4-amine (0.025 g, 0.057 mmol, 1.0 eq.) in 10 ml of ethanol, was added 5 ml of 1.23 M solution of HCl in ethanol. Solution was stirred at room temperature for 10 minutes then solvent was evaporated under reduced pressure at lower temperature. Similar procedure was repeated twice and then compound was submitted for lyophilisation to get 0.025 g of N-(2-(4-fluorophenyl)-5-isopropylpyrimidin-4-yl)-3-(piperazin-1-yl)-1H-pyrrolo [2, 3-b] pyridin-4-amine hydrochloride (Compound No. 1). LCMS (M+1): 432.2 ¹H NMR: (400 MHz, DMSO-d₆): δ 8.76 (s, 1H), 8.58 (d, J=6.85 Hz, 1H), 8.37-8.45 (m, 3H), 7.75 (s, 1H), 7.39 (t, J=8.80 Hz, 2H), 3.24 (br. s., 6H), 3.16-3.28 (m, J=6.36 Hz, 3H), 1.41 (d, J=6.85 Hz, 6H)

### Example 2: Preparation of N-(2-(4-fluorophenyl)-5-isopropylpyrimidin-4-yl)-3-(4-methylpiperazin-1-yl)-1H-pyrrolo [2, 3-b] pyridin-4-amine hydrochloride (Compound No. 2)

N-(2-(4-fluorophenyl)-5-isopropylpyrimidin-4-yl)-3-(4-methylpiperazin-1-yl)-1H-pyrrolo [2, 3-b] pyridin-4-amine hydrochloride (Compound No. 2) was prepared in accordance with steps illustrated in scheme 2 set out below:

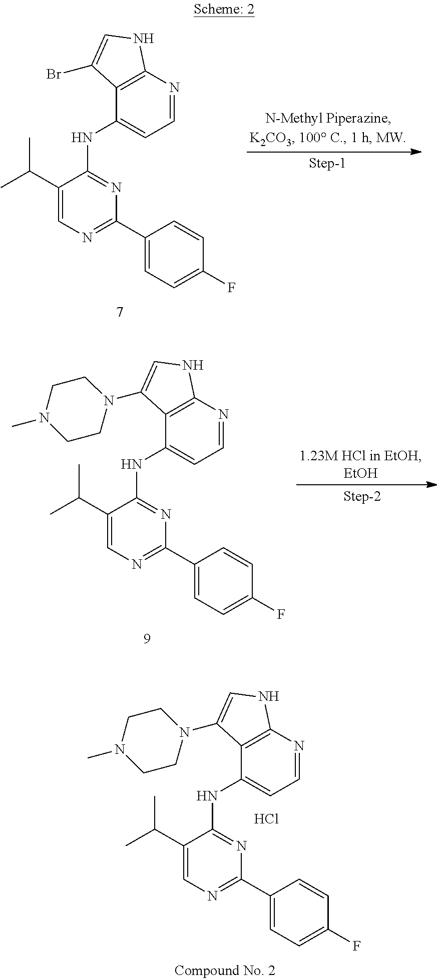 [SMILES: CC(C)c1cnc(-c2ccc(F)cc2)nc1Nc1ccnc2[nH]cc(Br)c12 ; CC(C)c1cnc(-c2ccc(F)cc2)nc1Nc1ccnc2[nH]cc(N3CCN(C)CC3)c12 ; CC(C)c1cnc(-c2ccc(F)cc2)nc1Nc1ccnc2[nH]cc(N3CCN(C)CC3)c12 ; Cl]

### Step 1: Synthesis of N-(2-(4-fluorophenyl)-5-isopropylpyrimidin-4-yl)-3-(piperidin-1-yl)-1H-pyrrolo [2,3-b] pyridin-4-amine

To a solution of 3-bromo-N-(2-(4-fluorophenyl)-5-isopropylpyrimidin-4-yl)-1H-pyrrolo[2,3-b]pyridin-4-amine (0.150 g, 0.351 mmol, 1.0 eq) in 6 ml of DMF, were added N-Methyl piperazine (0.176 g, 1.75 mmol, 5.0 eq) and K₂CO₃ (0.097 g, 0.702 mmol, 2.0 eq). Reaction mass was heated at 100° C. for 1 hour under microwave. After completion of reaction (LCMS monitoring), reaction mass was filtered to remove K₂CO₃ and filtrate was concentrated on high vacuum pump and crude was purified by PREP-HPLC to give 0.018 g of N-(2-(4-fluorophenyl)-5-isopropylpyrimidin-4-yl)-3-(piperidin-1-yl)-1H-pyrrolo [2, 3-b]pyridin-4-amine as white solid. LCMS (M+1): 446.3 ¹H NMR (400 MHz, DMSO-d₆): δ 11.47 (br. s., 1H), 9.73 (s, 1H), 8.58 (s, 1H), 8.42 (dd, J=5.92, 8.55 Hz, 2H), 8.32 (d, J=5.26 Hz, 1H), 8.19 (d, J=5.26 Hz, 1H), 7.49 (d, J=1.75 Hz, 1H), 7.38 (t, J=8.77 Hz, 2H), 3.11 (br. s., 5H), 2.79 (br. s., 4H), 1.39 (d, J=6.58 Hz, 6H)

### Step 2: Synthesis of N-(2-(4-fluorophenyl)-5-isopropylpyrimidin-4-yl)-3-(4-methylpiperazin-1-yl)-1H-pyrrolo [2,3-b] pyridin-4-amine hydrochloride

To the clear solution of N-(5-isopropyl-2-phenylpyrimidin-4-yl)-3-(4-methylpiperazin-1-yl)-1H-pyrrolo [2, 3-b] pyridin-4-amine (0.018 g, 0.040 mmol, 1.0 eq) in 10 ml of ethanol was added 5 ml of 1.23 M solution of HCl in ethanol. Solution was stirred at rt for 10 min then solvent was evaporated under reduced pressure at lower temperature. Similar procedure was repeated for 2 more times and then compound was submitted for lyophilisation to get 0.018 g of N-(2-(4-fluorophenyl)-5-isopropylpyrimidin-4-yl)-3-(4-methylpiperazin-1-yl)-1H-pyrrolo [2, 3-b] pyridin-4-amine hydrochloride (Compound No. 2). LCMS (M+1): 446.3 ¹H NMR (400 MHz, DMSO-d₆): δ 12.61 (br. s., 1H), 11.12 (br. s., 1H), 10.19 (br. s., 1H), 8.75 (s, 1H), 8.53 (br. s., 1H), 8.39-8.45 (m, 3H), 7.84 (br. s., 1H), 7.39 (t, J=8.80 Hz, 2H), 3.47 (br. s., 3H), 3.38 (br. s., 2H), 3.19-3.03 (m, 4H), 2.89 (d, J=3.91 Hz, 3H), 1.43 (d, J=6.85 Hz, 6H)

### Example 3: Preparation of N-(2-(4-fluorophenyl)-5-isopropylpyrimidin-4-yl)-3-(piperidin-1-yl)-1H-pyrrolo [2, 3-b] pyridin-4-amine hydrochloride (Compound No. 3)

N-(2-(4-fluorophenyl)-5-isopropylpyrimidin-4-yl)-3-(piperidin-1-yl)-1H-pyrrolo [2, 3-b] pyridin-4-amine hydrochloride (Compound No. 3) was prepared according to steps illustrated in scheme 3 set out below:

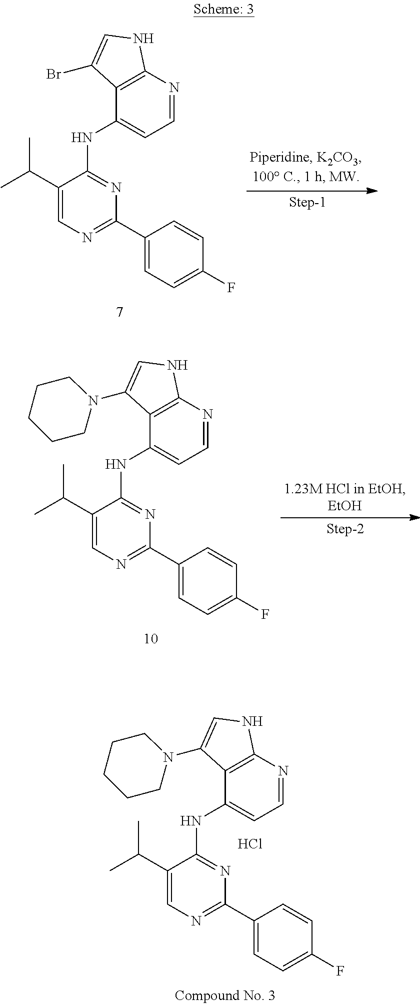 [SMILES: CC(C)c1cnc(-c2ccc(F)cc2)nc1Nc1ccnc2[nH]cc(Br)c12 ; CC(C)c1cnc(-c2ccc(F)cc2)nc1Nc1ccnc2[nH]cc(N3CCCCC3)c12 ; CC(C)c1cnc(-c2ccc(F)cc2)nc1Nc1ccnc2[nH]cc(N3CCCCC3)c12 ; Cl]

### Step 1: Synthesis of N-(2-(4-fluorophenyl)-5-isopropylpyrimidin-4-yl)-3-(piperidin-1-yl)-1H-pyrrolo [2,3-b] pyridin-4-amine

To a solution of 3-bromo-N-(2-(4-fluorophenyl)-5-isopropylpyrimidin-4-yl)-1H-pyrrolo[2,3-b]pyridin-4-amine (0.150 g, 0.352 mmol, 1.0 eq) in 6 ml of DMF, were added piperidine (0.150 g, 1.75 mmol, 5.0 eq) and K₂CO₃ (0.097 g, 0.704 mmol, 2.0 eq). Reaction mass was heated at 100° C. for 1 hour under microwave and it was monitored by LCMS. Reaction mixture was filtered and filtrate was concentrated on high vacuum pump and crude material was purified by PREP-HPLC to give 0.020 g of N-(2-(4-fluorophenyl)-5-isopropylpyrimidin-4-yl)-3-(piperidin-1-yl)-1H-pyrrolo [2, 3-b] pyridin-4-amine as pale white solid. LCMS (M+1): 431.3 ¹H NMR (400 MHz, DMSO-d<): δ 12.59 (br. s., 1H), 10.59 (br. s., 1H), 8.80 (s, 1H), 8.60 (d, J=7.02 Hz, 1H), 8.37-8.48 (m, 3H), 7.66 (br. s., 1H), 7.39 (t, J=8.44 Hz, 2H), 3.42-3.28 (m, 1H), 2.99 (br. s., 4H), 1.78 (br. s., 4H), 1.60 (br. s., 2H), 1.39 (d, J=6.58 Hz, 6H)

### Step 2: Synthesis of N-(2-(4-fluorophenyl)-5-isopropylpyrimidin-4-yl)-3-(piperidin-1-yl)-1H-pyrrolo [2,3-b] pyridin-4-amine hydrochloride

N-(5-isopropyl-2-phenylpyrimidin-4-yl)-3-(piperidin-1-yl)-1H-pyrrolo [2, 3-b]pyridin-4-amine (0.019 g, 0.044 mmol, 1.0 eq) was completely solubilized in 10 ml of ethanol and HCl in ethanol (5 ml of 1.23 M solution) was added to it. Solution was stirred at room temperature for 10 minutes then solvent was evaporated under reduced pressure at lower temperature. Similar procedure was repeated for 2 more times and then compound was submitted for lyophilization to get 0.021 g of N-(2-(4-fluorophenyl)-5-isopropylpyrimidin-4-yl)-3-(piperidin-1-yl)-1H-pyrrolo [2, 3-b] pyridin-4-amine hydrochloride (Compound No. 3).

LCMS (M+1): 431.3 ¹H NMR (400 MHz, DMSO-d₆): δ 12.27 (br. s., 1H), 10.46 (br. s., 1H), 8.76 (s, 1H), 8.55 (d, J=6.85 Hz, 1H), 8.44 (dd, J=5.62, 8.56 Hz, 2H), 8.37 (d, J=6.36 Hz, 2H), 7.61 (br. s., 1H), 7.40 (t, J=8.80 Hz, 2H), 2.99 (br. s., 5H), 1.70 (br. s., 4H), 1.61 (br. s., 2H), 1.39 (d, J=6.85 Hz, 6H).

### Example 4: Preparation of N-(2-(4-fluorophenyl)-5-isopropylpyrimidin-4-yl)-3-(2-methylpiperazin-1-yl)-1H-pyrrolo[2,3-b]pyridin-4-amine hydrochloride (Compound No. 4)

N-(2-(4-fluorophenyl)-5-isopropylpyrimidin-4-yl)-3-(2-methylpiperazin-1-yl)-1H-pyrrolo[2,3-b]pyridin-4-amine hydrochloride (Compound No. 4) was prepared according to steps of scheme 4 set out below:

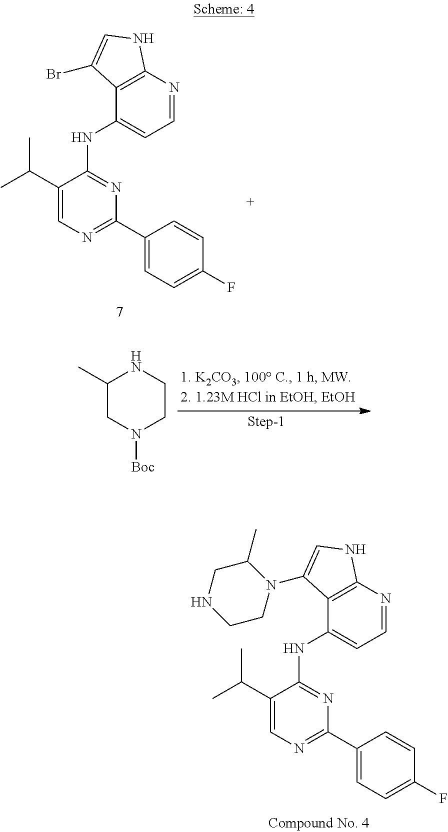 [SMILES: CC(C)c1cnc(-c2ccc(F)cc2)nc1Nc1ccnc2[nH]cc(Br)c12 ; CC1CN(C(=O)OC(C)(C)C)CCN1 ; CC(C)c1cnc(-c2ccc(F)cc2)nc1Nc1ccnc2[nH]cc(N3CCNCC3C)c12]

To a solution of 3-bromo-N-(2-(4-fluorophenyl)-5-isopropylpyrimidin-4-yl)-1H-pyrrolo[2,3-b]pyridin-4-amine (0.150 g, 0.352 mmol, 1.0 eq.) in 3 ml of DMF, was added tert-butyl 3-methylpiperazine-1-carboxylate (0.352 g, 1.76 mmol, 5.0 eq.) and K₂CO₃ (0.243 g, 1.76 mmol, 5.0 eq.). Reaction mass was heated at 100° C. for 1 hour under microwave and it was monitored by LCMS. Reaction mixture was filtered and filtrate was concentrated on high vacuum pump and crude material was purified by PREP-HPLC to give 60 mg of tert-butyl 4-(4-((2-(4-fluorophenyl)-5-isopropylpyrimidin-4-yl)amino)-1H-pyrrolo[2,3-b]pyridin-3-yl)-3-methylpiperazine-1-carboxylate as white solid. Desired product was characterized by LCMS.

Tert-butyl 4-(4-((2-(4-fluorophenyl)-5-isopropylpyrimidin-4-yl)amino)-1H-pyrrolo[2,3-b]pyridin-3-yl)-3-methylpiperazine-1-carboxylate (0.060 g, 0.11 mmol, 1.0 eq.) was solubilized in 2 ml of dioxane and dioxane HCl (5 ml of 4 M solution) was added to it. Solution was stirred at room temperature for 30 minutes then solvent was evaporated under reduced pressure at lower temperature. Similar procedure was repeated twice and then residue was submitted for lyophilization to get 0.030 g of N-(2-(4-fluorophenyl)-5-isopropylpyrimidin-4-yl)-3-(2-methylpiperazin-1-yl)-1H-pyrrolo[2,3-b]pyridin-4-amine hydrochloride (Compound No. 4). LCMS (M+1): 446.3 ¹H NMR (400 MHz, DMSO-d₆): δ 12.76 (br. s., 1H), 8.78 (s, 1H), 8.68 (d, J=6.65 Hz, 1H), 8.40-8.50 (m, 3H), 7.72 (br. s., 1H), 7.40 (t, J=8.80 Hz, 3H), 3.62-3.72 (m, 3H), 3.35-3.50 (m, 4H), 3.17 (m, 3H), 2.66-2.71 (m, 1H), 1.43 (d, J=18.00 Hz, 3H), 1.45 (d, J=18.00 Hz, 3H)

### Example 5: Preparation of N-(2-(4-fluorophenyl)-5-isopropylpyrimidin-4-yl)-3-morpholino-1H-pyrrolo [2,3-b] pyridin-4-amine hydrochloride (Compound No. 5)

N-(2-(4-fluorophenyl)-5-isopropylpyrimidin-4-yl)-3-morpholino-1H-pyrrolo [2, 3-b] pyridin-4-amine hydrochloride (Compound No. 5) was prepared according to steps illustrated in scheme 5 set out below:

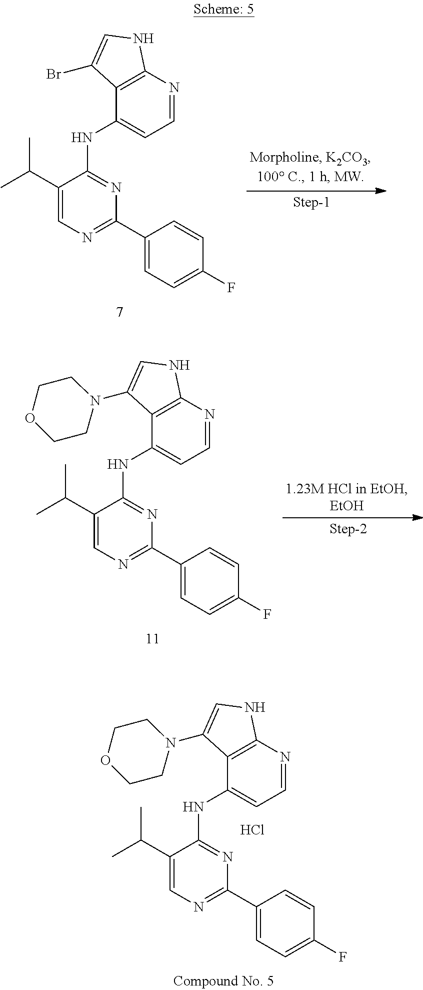 [SMILES: CC(C)c1cnc(-c2ccc(F)cc2)nc1Nc1ccnc2[nH]cc(Br)c12 ; CC(C)c1cnc(-c2ccc(F)cc2)nc1Nc1ccnc2[nH]cc(N3CCOCC3)c12 ; CC(C)c1cnc(-c2ccc(F)cc2)nc1Nc1ccnc2[nH]cc(N3CCOCC3)c12 ; Cl]

### Step 1: Synthesis of N-(2-(4-fluorophenyl)-5-isopropylpyrimidin-4-yl)-3-morpholino-1H-pyrrolo [2,3-b] pyridin-4-amine

To a solution of 3-bromo-N-(2-(4-fluorophenyl)-5-isopropylpyrimidin-4-yl)-1H-pyrrolo[2,3-b]pyridin-4-amine (0.100 g, 0.235 mmol, 1.0 eq) in 3 ml of DMF, were added Morpholine (0.102 ml, 1.17 mmol, 5.0 eq) and K₂CO₃ (0.064 g, 0.470 mmol, 2.0 eq). Reaction was heated at 100° C. for 1 hour under microwave and it was monitored by LCMS. Reaction mixture was filtered and filtrate was concentrated on high vacuum pump. Crude material was purified by PREP-HPLC to give 0.022 g of N-(2-(4-fluorophenyl)-5-isopropylpyrimidin-4-yl)-3-morpholino-1H-pyrrolo [2, 3-b] pyridin-4-amine. ¹H NMR (400 MHz, DMSO-d6): δ 9.94 (br. s., 1H), 8.65 (s, 1H), 8.44 (br. s., 1H), 8.42 (d, 7=5.48 Hz, 2H), 8.26 (d, 7=5.70 Hz, 1H), 7.58 (br. s., 2H), 7.39 (t, 7=8.77 Hz, 3H), 3.77 (br. s., 4H), 3.17 (s, 1H), 3.02 (br. s., 4H), 1.40 (d, 7=7.02 Hz, 6H)

### Step 2: Synthesis of N-(2-(4-fluorophenyl)-5-isopropylpyrimidin-4-yl)-3-morpholino-1H-pyrrolo [2,3-b] pyridin-4-amine hydrochloride

N-(2-(4-fluorophenyl)-5-isopropylpyrimidin-4-yl)-3-morpholino-1H-pyrrolo [2, 3-b] pyridin-4-amine (0.022 g, 0.050 mmol, 1.0 eq) was completely solubilized in ethanol (10 ml) and HCl in ethanol (5 ml of 1.23 M solution) was added to it. Reaction was stirred at room temperature for 10 minutes then solvent was evaporated under reduced pressure at lower temperature. Above procedure was repeated twice, solvent was evaporated and residue was lyophilized to get 0.022 g of N-(2-(4-fluorophenyl)-5-isopropylpyrimidin-4-yl)-3-morpholino-1H-pyrrolo [2, 3-b] pyridin-4-amine hydrochloride (Compound No. 5). LCMS (M+1): 433.3 ¹H NMR (400 MHz, DMSO-d₆): δ 12.56 (br. s., 1H), 10.31 (br. s., 1H), 8.78 (s, 1H), 8.59 (d, 7=7.34 Hz, 1H), 8.38-8.47 (m, 3H), 7.74 (s, 1H), 7.39 (t, 7=8.80 Hz, 3H), 3.72 (br. s., 3H), 3.36-3.44 (m, 2H), 3.05 (br. s., 5H), 1.41 (d, 7=6.85 Hz, 6H)

### Example 6: Preparation of N-(2-(4-fluorophenyl)-5-isopropylpyrimidin-4-yl)-3-(1H-1, 2, 4-triazol-1-yl)-1H-pyrrolo [2, 3-b] pyridin-4-amine hydrochloride (Compound No. 6)

N-(2-(4-fluorophenyl)-5-isopropylpyrimidin-4-yl)-3-(1H-1, 2, 4-triazol-1-yl)-1H-pyrrolo [2, 3-b] pyridin-4-amine hydrochloride (Compound No. 6) was prepared according to steps illustrated in scheme 6 set out below:

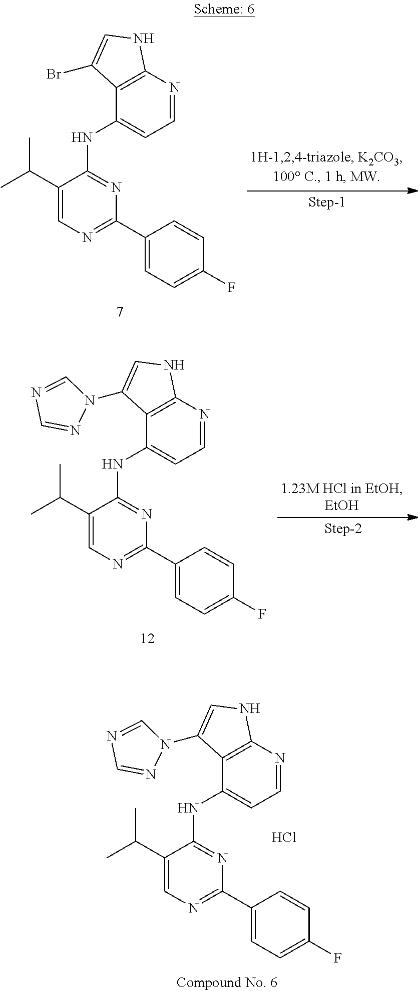 [SMILES: CC(C)c1cnc(-c2ccc(F)cc2)nc1Nc1ccnc2[nH]cc(Br)c12 ; CC(C)c1cnc(-c2ccc(F)cc2)nc1Nc1ccnc2[nH]cc(-n3cncn3)c12 ; CC(C)c1cnc(-c2ccc(F)cc2)nc1Nc1ccnc2[nH]cc(-n3cncn3)c12 ; Cl]

### Step 1: Synthesis of N-(2-(4-fluorophenyl)-5-isopropylpyrimidin-4-yl)-3-(1H-1,2,4-triazol-1-yl)-1H-pyrrolo [2,3-b] pyridin-4-amine

To a solution of 3-bromo-N-(2-(4-fluorophenyl)-5-isopropylpyrimidin-4-yl)-1H-pyrrolo[2,3-b]pyridin-4-amine (0.100 g, 0.235 mmol, 1.0 eq) in 10 ml of DMF, were added 1H-1,2,4-triazole (0.162 g, 1.17 mmol, 5.0 eq) and K₂CO₃ (0.064 g, 0.470 mmol, 2.0 eq). Reaction was heated at 100° C. for 1 hour under microwave and it was monitored by LCMS. Reaction mixture was filtered and filtrate was concentrated on high vacuum pump. Crude material was purified by PREP-HPLC to give 0.025 g of N-(2-(4-fluorophenyl)-5-isopropylpyrimidin-4-yl)-3-(1H-1, 2, 4-triazol-1-yl)-1H-pyrrolo [2, 3-b] pyridin-4-amine. LCMS (M+1): 415.3

### Step 2: Synthesis of N-(2-(4-fluorophenyl)-5-isopropylpyrimidin-4-yl)-3-(1H-1,2,4-triazol-1-yl)-1H-pyrrolo [2,3-b] pyridin-4-amine hydrochloride

N-(2-(4-fluorophenyl)-5-isopropylpyrimidin-4-yl)-3-(1H-1, 2, 4-triazol-1-yl)-1H-pyrrolo [2, 3-b] pyridin-4-amine (0.025 g, 0.060 mmol, 1.0 eq) was completely solubilized in ethanol (10 ml) and HCl in ethanol (5 ml of 1.23 M solution) was added to it. Reaction was stirred at room temperature for 10 minutes then solvent was evaporated under reduced pressure at lower temperature. Above procedure was repeated twice, solvent was evaporated and residue was lyophilized to get 0.020 g of N-(2-(4-fluorophenyl)-5-isopropylpyrimidin-4-yl)-3-(1H-1, 2, 4-triazol-1-yl)-1H-pyrrolo [2, 3-b] pyridin-4-amine hydrochloride (Compound No. 6). LCMS (M+1): 415.3 ¹H NMR (400 MHz, DMSO-d₆): δ 12.52 (br. s., 1H), 8.91 (br. s., 1H), 8.48 (s, 1H), 8.40 (d, J=5.87 Hz, 1H), 8.31 (s, 1H), 8.24 (d, J=5.87 Hz, 1H), 8.21 (br. s., 2H), 8.08 (d, J=1.96 Hz, 1H), 7.35 (t, J=9.00 Hz, 2H), 3.17-3.23 (m, 1H), 1.33 (d, J=7.04 Hz, 6H).

### Example 7 and 8: Preparation of N-(2-(4-fluorophenyl)-5-isopropylpyrimidin-4-yl)-3-(2-methylpiperazin-1-yl)-1H-pyrrolo [2, 3-b] pyridin-4-amine hydrochloride (Compound No. 7) and N-(2-(4-fluorophenyl)-5-isopropylpyrimidin-4-yl)-3-(2-methylpiperazin-1-yl)-1H-pyrrolo [2, 3-b] pyridin-4-amine hydrochloride (Compound No. 8)

N-(2-(4-fluorophenyl)-5-isopropylpyrimidin-4-yl)-3-(2-methylpiperazin-1-yl)-1H-pyrrolo [2, 3-b] pyridin-4-amine hydrochloride (Compound No. 7) and N-(2-(4-fluorophenyl)-5-isopropylpyrimidin-4-yl)-3-(2-methylpiperazin-1-yl)-1H-pyrrolo [2, 3-b]pyridin-4-amine hydrochloride (Compound No. 8) was prepared according to steps of scheme 8 set out below:

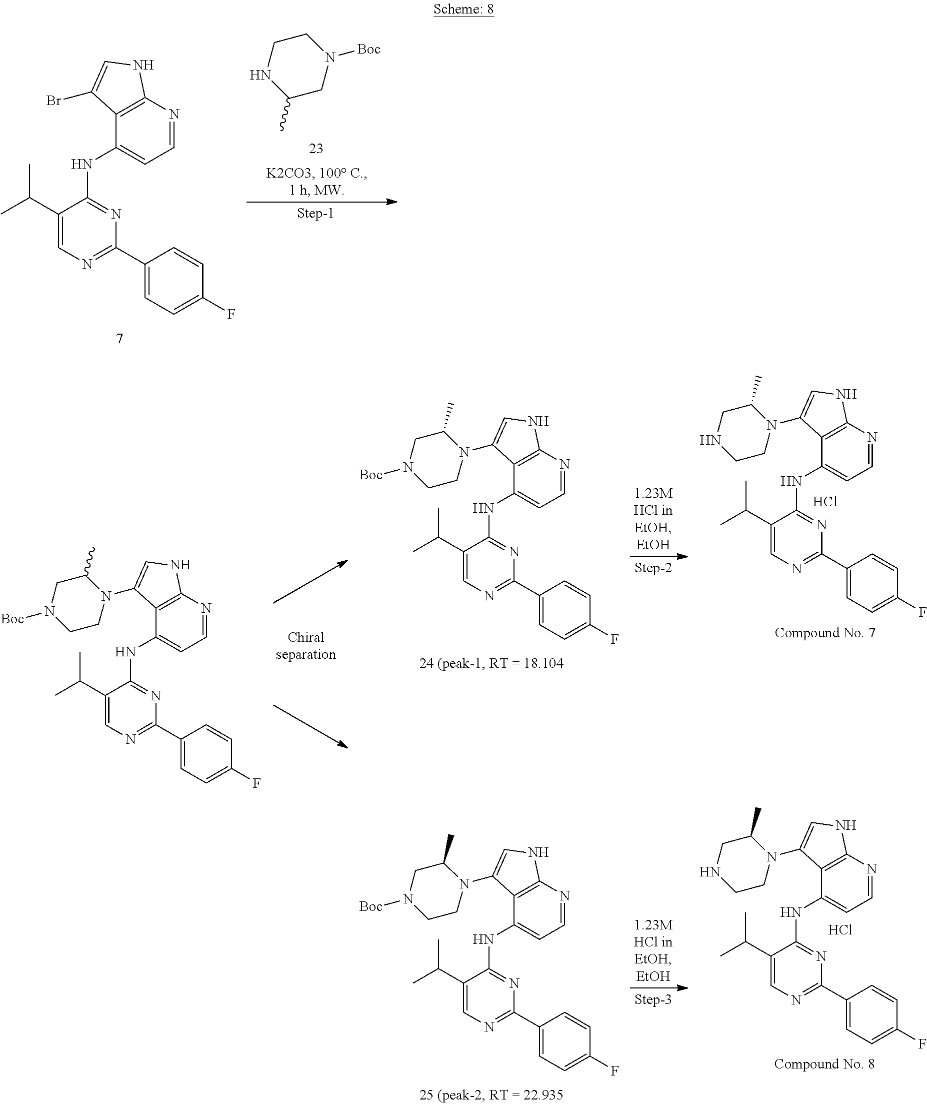

### Step 1: Synthesis of tert-butyl 4-(4-(2-(4-fluorophenyl)-5-isopropylpyrimidin-4-ylamino)-1H-pyrrolo [2, 3-b] pyridin-3-yl)-3-methylpiperazine-1-carboxylate

To a solution of 3-bromo-N-(2-(4-fluorophenyl)-5-isopropylpyrimidin-4-yl)-1H-pyrrolo[2,3-b]pyridin-4-amine (0.100 g, 0.235 mmol, 1.0 eq) in 4 ml of DMF, were added tert-butyl 3-methylpiperazine-1-carboxylate (0.324 mg, 1.17 mmol, 5.0 eq) and K₂CO₃ (0.162 g, 1.17 mmol, 5.0 eq). Reaction was heated at 100° C. for 1 hour under microwave and it was monitored by LCMS. Reaction mixture was filtered and filtrate was concentrated on high vacuum pump. Crude material was purified by CHIRAL-HPLC to give two enantiomers, Peak-1=10 mg (RT=18.104) and Peak-2=15 mg (RT=22.925). LCMS (M+1): 426.1 ¹H NMR (400 MHz, DMSO-d₆): δ 11.54 (br. s., 1H), 9.95 (s, 1H), 8.60 (s, 1H), 8.39-8.46 (m, 3H), 8.19 (d, J=5.70 Hz, 1H), 7.56 (d, J=2.96 Hz, 1H), 7.39 (t, J=8.88 Hz, 2H), 3.21 (br. s., 2H), 2.93 (s, 2H), 1.45 (s, 9H), 1.37 (dd, J=6.85, 11.56 Hz, 6H), 0.88 (d, J=6.14 Hz, 3H) (peak-1)

### Step 2: Synthesis of N-(2-(4-fluorophenyl)-5-isopropylpyrimidin-4-yl)-3-(2-methylpiperazin-1-yl)-1H-pyrrolo [2,3-b] pyridin-4-amine hydrochloride

Tert-butyl 4-(4-(2-(4-fluorophenyl)-5-isopropylpyrimidin-4-ylamino)-1H-pyrrolo [2, 3-b] pyridin-3-yl)-3-methylpiperazine-1-carboxylate (0.010 g, 0.018 mmol, 1.0 eq, compound-24) was completely solubilized in ethanol (10 ml) and HCl in ethanol (5 ml of 1.23 M solution) was added to it. Reaction was stirred at room temperature for 10 minutes then solvent was evaporated under reduced pressure at lower temperature. Above procedure was repeated twice, solvent was evaporated and residue was lyophilized to get 8 mg of N-(2-(4-fluorophenyl)-5-isopropylpyrimidin-4-yl)-3-(2-methylpiperazin-1-yl)-1H-pyrrolo [2, 3-b]pyridin-4-amine hydrochloride (Compound No. 7). LCMS (M+1): 426.1 ¹H NMR (400 MHz, DMSO-d₆): δ 10.04 (s, 1H), 9.74 (s, 1H), 9.45 (s, 1H), 8.75 (s, 1H), 8.63 (d, J=6.14 Hz, 1H), 8.45 (dd, J=5.59, 8.88 Hz, 3H), 8.39 (d, J=6.03 Hz, 1H), 7.69 (br. s., 1H), 7.40 (t, J=8.93 Hz, 3H), 3.70-3.77 (m, 2H), 3.37 (br. s., 2H), 3.01 (br. s., 2H), 2.94-2.87 (m, 2H), 1.46 (d, J=6.69 Hz, 3H), 1.42 (d, J=6.36 Hz, 3H), 0.93 (d, J=5.92 Hz, 3H)

### Step 3: Synthesis of N-(2-(4-fluorophenyl)-5-isopropylpyrimidin-4-yl)-3-(2-methylpiperazin-1-yl)-1H-pyrrolo [2,3-b] pyridin-4-amine hydrochloride

Tert-butyl 4-(4-(2-(4-fluorophenyl)-5-isopropylpyrimidin-4-ylamino)-1H-pyrrolo [2, 3-b] pyridin-3-yl)-3-methylpiperazine-1-carboxylate (0.015 g, 0.027 mmol, 1.0 eq, compound-25) was completely solubilized in ethanol (10 ml) and HCl in ethanol (5 ml of 1.23 M solution) was added to it. Reaction was stirred at room temperature for 10 min then solvent was evaporated under reduced pressure at lower temperature. Above procedure was repeated for twice, solvent was evaporated and residue was lyophilized to get 7 mg of N-(2-(4-fluorophenyl)-5-isopropylpyrimidin-4-yl)-3-(2-methylpiperazin-1-yl)-1H-pyrrolo [2, 3-b]pyridin-4-amine hydrochloride (Compound No. 8). LCMS (M+1): 426.1 ¹H NMR (400 MHz, DMSO-d₆): δ 12.46 (s, 1H), 10.00 (s, 1H), 9.77 (s, 1H), 9.44 (s, 1H), 8.73 (s, 1H), 8.61 (d, J=6.36 Hz, 1H), 8.44 (dd, J=5.65, 8.82 Hz, 3H), 8.38 (d, J=6.03 Hz, 1H), 7.67 (br. s., 1H), 7.40 (t, J=8.82 Hz, 3H), 3.17 (br. s., 3H), 3.00 (d, J=13.37 Hz, 2H), 1.44 (d, J=10.19 Hz, 3H), 1.39-1.49 (m, 3H), 0.92 (d, J=6.14 Hz, 3H).

### Example 9: Preparation of (R)—N-(2-(4-fluorophenyl)-5-isopropylpyrimidin-4-yl)-3-(3-methylpiperazin-1-yl)-1H-pyrrolo[2,3-b]pyridin-4-amine hydrochloride (Compound No. 9)

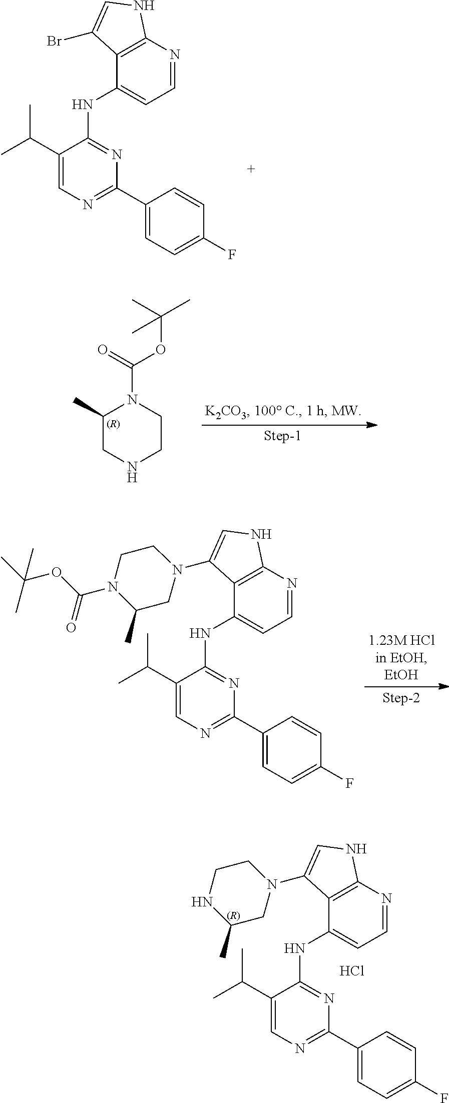

### Step 1: Synthesis of (R)-tert-butyl 4-(4-(2-(4-fluorophenyl)-5-isopropylpyrimidin-4-ylamino)-1H-pyrrolo[2,3-b]pyridin-3-yl)-2-methylpiperazine-1-carboxylate

To solution of 3-bromo-N-(2-(4-fluorophenyl)-5-isopropylpyrimidin-4-yl)-1H-pyrrolo[2,3-b]pyridin-4-amine (0.100 g, 0.234 mmol, 1.0 eq) in 4 ml of DMF were added (R)-tert-butyl 2-methylpiperazine-1-carboxylate (0.234 g, 1.17 mmol, 5.0 eq) and K₂CO₃ (0.162 g, 1.17 mmol, 5.0 eq). Reaction was heated at 100° C. for 1 h under microwave and it was monitored by LCMS. Reaction mixture was filtered and filtrate was concentrated on high vacuum pump. Crude material was purified by PREP-HPLC to give 0.020 g of (R)—N-(2-(4-fluorophenyl)-5-isopropylpyrimidin-4-yl)-3-(3-methylpiperazin-1-yl)-1H-pyrrolo[2,3-b]pyridin-4-amine. LCMS (M+1): 546.3 ¹H NMR (400 MHz, DMSO-d₆): δ 11.54 (br. s., 1H), 9.18 (br. s., 1H), 8.62 (s, 1H), 8.35 (dd, J=5.54, 8.61 Hz, 2H), 8.20 (d, J=5.92 Hz, 1H), 8.04 (d, J=5.48 Hz, 1H), 7.36 (s, 1H), 7.31-7.35 (m, 2H), 4.24 (br. s., 1H), 3.79 (d, J=14.36 Hz, 1H), 3.01-3.09 (m, 2H), 2.98 (br. s., 2H), 1.41 (s, 9H), 1.35 (d, J=8.77 Hz, 3H), 1.31-1.38 (m, 3H), 1.18 (d, J=6.80 Hz, 3H)

### Step 2: Synthesis of (R)—N-(2-(4-fluorophenyl)-5-isopropylpyrimidin-4-yl)-3-(3-methyl piperazin-1-yl)-1H-pyrrolo[2,3-b]pyridin-4-amine hydrochloride

(R)—N-(2-(4-fluorophenyl)-5-isopropylpyrimidin-4-yl)-3-(3-methylpiperazin-1-yl)-1H-pyrrolo[2,3-b]pyridin-4-amine (0.020 g, 0.036 mmol, 1.0 eq) was completely solubilized in ethanol (10 ml) and HCl in ethanol (5 ml of 1.23 M solution) was added to it. Reaction was stirred at room temperature for 10 minutes then solvent was evaporated under reduced pressure at lower temperature. Above procedure was repeated for twice, solvent was evaporated and residue was lyophilized to get 0.015 g of Synthesis of (R)—N-(2-(4-fluorophenyl)-5-isopropylpyrimidin-4-yl)-3-(3-methylpiperazin-1-yl)-1H-pyrrolo[2,3-b]pyridin-4-amine hydrochloride (Compound No. 9). LCMS (M+1): 446.2 ¹H NMR (400 MHz, DMSO-d₆): δ 12.51 (br. s., 1H), 10.02 (br. s., 1H), 9.63 (br. s., 1H), 9.20 (br. s., 1H), 8.74 (s, 1H), 8.51 (br. s., 1H), 8.37-8.44 (m, 3H), 7.69 (br. s., 1H), 7.39 (t, J=8.82 Hz, 2H), 3.48-2.39 (m, 2H), 3.39-3.30 (m, 2H), 3.13-3.19 (m, 2H), 3.12-3.01 (m, 2H), 1.41 (d, J=6.91 Hz, 6H), 1.26 (d, 7=6.14 Hz, 3H).

### Example 10: Preparation of 4-(4-(2-(4-fluorophenyl)-5-isopropylpyrimidin-4-ylamino)-1H-pyrrolo [2, 3-b] pyridin-3-yl) piperazin-2-one formate (Compound No. 10)

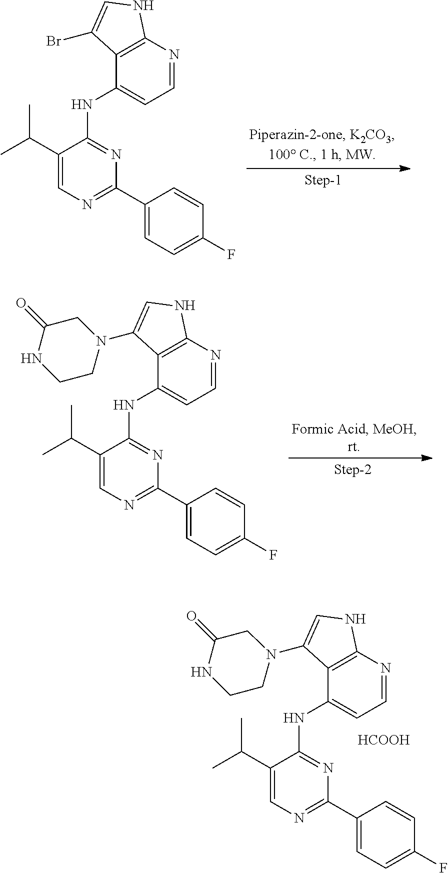 [SMILES: CC(C)c1cnc(-c2ccc(F)cc2)nc1Nc1ccnc2[nH]cc(Br)c12 ; CC(C)c1cnc(-c2ccc(F)cc2)nc1Nc1ccnc2[nH]cc(N3CCNC(=O)C3)c12 ; CC(C)c1cnc(-c2ccc(F)cc2)nc1Nc1ccnc2[nH]cc(N3CCNC(=O)C3)c12 ; "HCOOH"]

### Step 1: Synthesis of 4-(4-(2-(4-fluorophenyl)-5-isopropylpyrimidin-4-ylamino)-1H-pyrrolo [2, 3-b] pyridin-3-yl) piperazin-2-one

To a solution of 3-bromo-N-(2-(4-fluorophenyl)-5-isopropylpyrimidin-4-yl)-1H-pyrrolo[2,3-b]pyridin-4-amine (0.100 g, 0.235 mmol, 1.0 eq) in 4 ml of DMF, were added piperazin-2-one (0.117 g, 1.17 mmol, 5.0 eq) and K₂CO₃ (0.064 g, 0.470 mmol, 2.0 eq). Reaction was heated at 100° C. for 1 hour under microwave and it was monitored by LCMS. Reaction mixture was filtered and filtrate was concentrated on high vacuum pump. Crude material was purified by PREP-HPLC to give 15 mg of 4-(4-(2-(4-fluorophenyl)-5-isopropylpyrimidin-4-ylamino)-1H-pyrrolo [2, 3-b]pyridin-3-yl) piperazin-2-one. LCMS (M+1): 446.2 ¹H NMR (400 MHz, DMSO-d₆): δ 11.51 (d, J=2.30 Hz, 1H), 9.91 (s, 1H), 8.54 (s, 1H), 8.38-8.46 (m, 4H), 8.21 (d, J=5.48 Hz, 1H), 8.10 (s, 1H), 7.48 (d, J=2.63 Hz, 1H), 7.39 (t, J=8.82 Hz, 2H), 3.54 (s, 2H), 3.35-3.38 (m, 2H), 3.23-3.28 (m, 3H), 3.03-3.10 (m, 1H), 1.33 (d, J=6.69 Hz, 6H).

### Step 2: Synthesis of 4-(4-(2-(4-fluorophenyl)-5-isopropylpyrimidin-4-ylamino)-1H-pyrrolo [2, 3-b] pyridin-3-yl) piperazin-2-one formate

To a solution of 4-(4-(2-(4-fluorophenyl)-5-isopropylpyrimidin-4-ylamino)-1H-pyrrolo [2, 3-b] pyridin-3-yl) piperazin-2-one (0.010 g, 0.022 mmol, 1.0 eq) in 10 ml of Methanol, was added 1 ml formic acid. Solution was stirred for 15 min and then solvent was evaporated and residue was lyophilized to get 10 mg of 4-(4-(2-(4-fluorophenyl)-5-isopropylpyrimidin-4-ylamino)-1H-pyrrolo [2, 3-b] pyridin-3-yl) piperazin-2-one formate (Compound No. 10). LCMS (M+1): 446.2 ¹H NMR (400 MHz, DMSO-d₆): δ 11.51 (d, J=2.74 Hz, 1H), 9.90 (s, 1H), 8.54 (s, 1H), 8.44 (dd, J=5.48, 9.00 Hz, 2H), 8.39 (d, J=5.48 Hz, 1H), 8.21 (d, J=5.48 Hz, 1H), 8.10 (br. s., 1H), 7.39 (t, J=8.80 Hz, 2H), 3.54 (s, 2H), 3.36 (br. s., 2H), 3.23-3.27 (m, 2H), 3.10-3.05 (m, 1H), 1.33 (d, 7=6.65 Hz, 6H)

### Example 11: Preparation of (S)—N-(2-(4-fluorophenyl)-5-isopropylpyrimidin-4-yl)-3-(3-methylpiperazin-1-yl)-1H-pyrrolo[2,3-b]pyridin-4-amine hydrochloride (Compound No. 11)

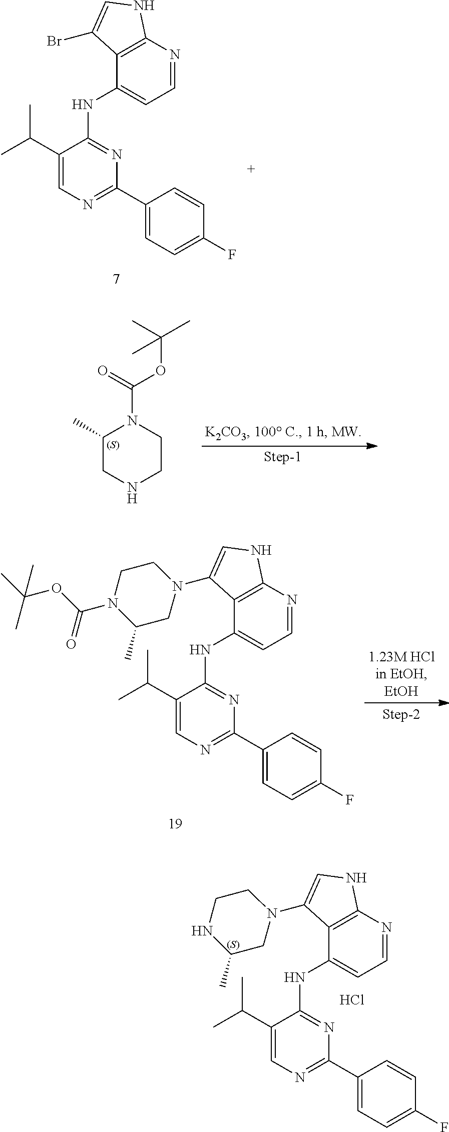

### Step 1: Synthesis of (S)-tert-butyl 4-(4-(2-(4-fluorophenyl)-5-isopropylpyrimidin-4-ylamino)-1H-pyrrolo[2,3-b]pyridin-3-yl)-2-methylpiperazine-1-carboxylate

To a solution of 3-bromo-N-(2-(4-fluorophenyl)-5-isopropylpyrimidin-4-yl)-1H-pyrrolo[2,3-b]pyridin-4-amine (0.100 g, 0.23 mmol, 1.0 eq) in 3 ml of DMF, were added (S)-tert-butyl 2-methylpiperazine-1-carboxylate (0.234 g, 1.17 mmol, 5.0 eq) and K₂CO₃ (0.162 g, 1.17 mmol, 5.0 eq). Reaction was heated at 100° C. for 1 hour under microwave and it was monitored by LCMS. Reaction mixture was filtered and filtrate was concentrated on high vacuum pump. Crude material was purified by SFC to give 0.069 g of (S)—N-(2-(4-fluorophenyl)-5-isopropylpyrimidin-4-yl)-3-(3-methylpiperazin-1-yl)-1H-pyrrolo[2,3-b]pyridin-4-amine. LCMS (M+1): 546.3 ¹H NMR (400 MHz, DMSO-d₆): δ 12.05 (br. s., 1H), 9.58 (br. s., 1H), 8.72 (s, 1H), 8.35-8-8.28 (m, 3H), 8.20-8.16 (m, 1H), 7.55 (s, 1H), 7.38-7.32 (t, J=8.82 Hz, 2H), 4.24 (br. s., 1H), 3.79 (d, J=14.36 Hz, 1H), 3.01-3.09 (m, 2H), 2.98 (br. s., 2H), 1.41 (s, 9H), 1.35 (d, J=8.77 Hz, 3H), 1.28-1.53 (m, 3H), 1.04-1.28 (m, 9H)

### Step 2: Synthesis of (S)—N-(2-(4-fluorophenyl)-5-isopropylpyrimidin-4-yl)-3-(3-methylpiperazin-1-yl)-1H-pyrrolo[2,3-b]pyridin-4-amine hydrochloride

To a solution of tert-butyl (S)-4-(4-((2-(4-fluorophenyl)-5-isopropylpyrimidin-4-yl)amino)-1H-pyrrolo[2,3-b]pyridin-3-yl)-2-methylpiperazine-1-carboxylate (0.067 g, 0.123 mmol, 1.0 eq.) in 2 ml of dioxane, was added 2 ml 4 M of dioxane in HCl. Solution was stirred for 15 minutes and then solvent was evaporated and this procedure was repeated twice. Solvent was evaporated and residue was lyophilized to get 35 mg of (S)—N-(2-(4-fluorophenyl)-5-isopropylpyrimidin-4-yl)-3-(3-methylpiperazin-1-yl)-1H-pyrrolo[2,3-b]pyridin-4-amine hydrochloride (Compound No. 11). LCMS (M+1): 446.1 ¹H NMR (400 MHz, DMSO-d₆): δ 12.73 (br. s., 1H), 10.14 (br. s., 1H), 9.86 (br. s., 1H), 9.39 (br. s., 1H), 8.76 (s, 1H), 8.57 (d, J=6.58 Hz, 1H), 8.37-8.45 (m, 3H), 7.72 (br. s., 1H), 7.36-7.43 (m, 2H), 3.43-3.51 (m, 2H), 3.29-3.40 (m, 2H), 3.13-3.22 (m, 2H), 3.01-3.08 (m, 2H), 1.42 (d, J=6.80 Hz, 6H), 1.27 (d, J=6.25 Hz, 3H).

### Example 12: Preparation of 1-(4-((2-(4-fluorophenyl)-5-isopropylpyrimidin-4-yl)amino)-1H-pyrrolo[2,3-b]pyridin-3-yl)piperidine-4-carboxamide formic acid (Compound No. 12)

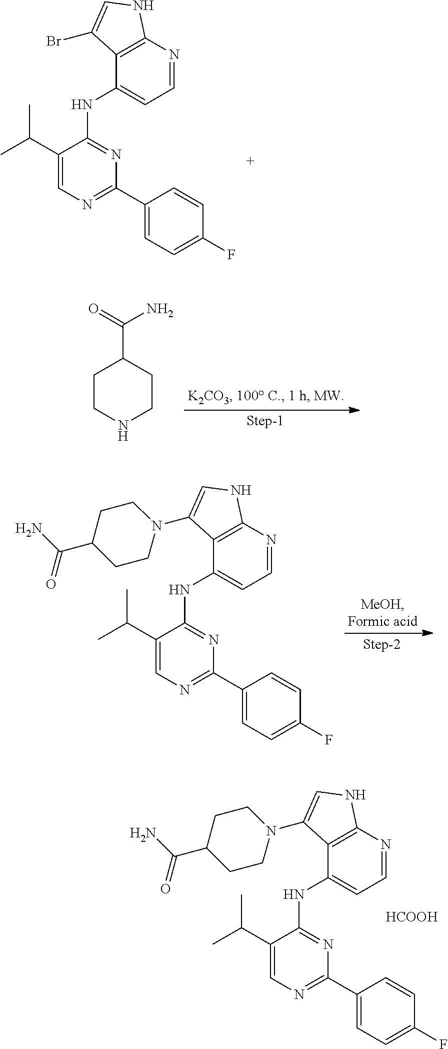 [SMILES: CC(C)c1cnc(-c2ccc(F)cc2)nc1Nc1ccnc2[nH]cc(Br)c12 ; NC(=O)C1CCNCC1 ; CC(C)c1cnc(-c2ccc(F)cc2)nc1Nc1ccnc2[nH]cc(N3CCC(C(N)=O)CC3)c12 ; CC(C)c1cnc(-c2ccc(F)cc2)nc1Nc1ccnc2[nH]cc(N3CCC(C(N)=O)CC3)c12 ; "HCOOH"]

### Step 1: Synthesis of 1-(4-((2-(4-fluorophenyl)-5-isopropylpyrimidin-4-yl)amino)-1H-pyrrolo[2,3-b]pyridin-3-yl)piperidine-4-carboxamide

To a solution of 3-bromo-N-(2-(4-fluorophenyl)-5-isopropylpyrimidin-4-yl)-1H-pyrrolo[2,3-b]pyridin-4-amine (0.15 g, 0.35 mmol, 1.0 eq.) in 4 ml of DMF, was added piperidine-4-carboxamide (0.225 g, 1.76 mmol, 5.0 eq.) and K₂CO₃ (0.243 g, 1.76 mmol, 5.0 eq.). Reaction was heated at 100° C. for 1 hour under microwave and it was monitored by LCMS. Reaction mixture was filtered and filtrate was concentrated on high vacuum pump. Crude material was purified by PREP-HPLC to give 28 mg of 1-(4-((2-(4-fluorophenyl)-5-isopropylpyrimidin-4-yl)amino)-1H-pyrrolo[2,3-b]pyridin-3-yl)piperidine-4-carboxamide. LCMS (M+1): 474.3 1H NMR (400 MHz, DMSO-d6): δ 12.31 (br. s., 1H), 10.43 (br. s., 1H), 8.74 (s, 1H), 8.63 (d, J=7.34 Hz, 1H), 8.38-8.46 (m, 3H), 7.66 (s, 1H), 7.34-7.42 (m, 3H), 6.81 (br. s., 1H), 3.35-3.40 (m, 1H), 3.26 (d, J=11.74 Hz, 2H), 2.80 (t, J=8.80 Hz, 2H), 2.33 (br. s., 1H), 1.82-1.86 (m, 4H), 1.40 (d, J=6.85 Hz, 6H).

### Step 2: Synthesis of 1-(4-((2-(4-fluorophenyl)-5-isopropylpyrimidin-4-yl)amino)-1H-pyrrolo[2,3-b]pyridin-3-yl)piperidine-4-carboxamide formic acid

To a solution of 1-(4-((2-(4-fluorophenyl)-5-isopropylpyrimidin-4-yl)amino)-1H-pyrrolo[2,3-b]pyridin-3-yl)piperidine-4-carboxamide (0.008 g, 0.123 mmol, 1.0 eq.) in 2 ml of methanol, was added 2 ml formic acid solution and reaction was stirred for 15 minutes and then solvent was evaporated and this procedure was repeated once. Solvent was evaporated and residue was lyophilized to get 7 mg 1-(4-((2-(4-fluorophenyl)-5-isopropylpyrimidin-4-yl)amino)-1H-pyrrolo[2,3-b]pyridin-3-yl)piperidine-4-carboxamide formic acid (Compound No. 12). LCMS (M+1): 474.1 ¹H NMR (400 MHz, DMSO-d₆): δ 11.39 (br. s., 1H), 9.94 (s, 1H), 8.57 (s, 1H), 8.44 (dd, J=5.87, 9.00 Hz, 2H), 8.38 (d, J=5.48 Hz, 2H), 8.17 (d, J=5.48 Hz, 1H), 7.32-7.43 (m, 4H), 6.79 (br. s., 1H), 3.21-3.27 (m, 1H), 2.76 (m, 3H), 2.67 (br. s., 1H), 2.30-2.36 (m, 1H), 1.83 (m, 4H), 1.38 (d, J=6.65 Hz, 6H).

### Example 13: Preparation of 4-(4-((2-(4-fluorophenyl)-5-isopropylpyrimidin-4-yl)amino)-1H-pyrrolo[2,3-b]pyridin-3-yl)piperidin-4-ol hydrochloride (Compound No. 13)

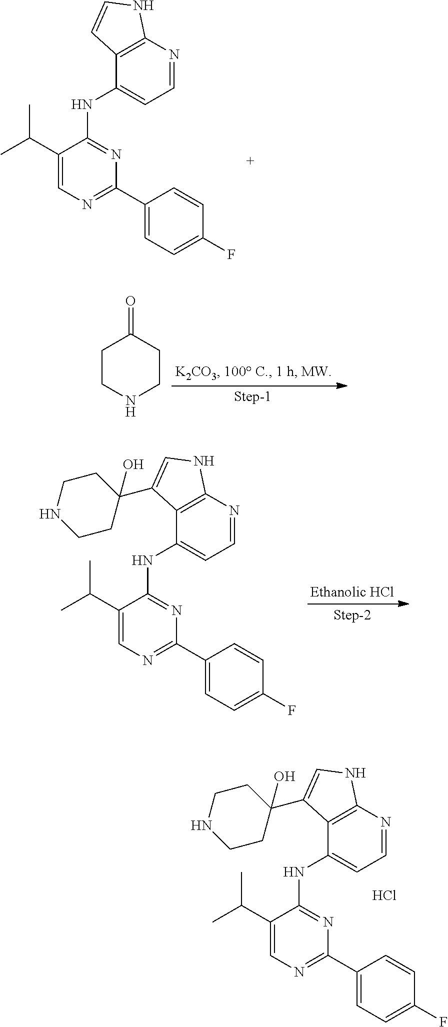 [SMILES: CC(C)c1cnc(-c2ccc(F)cc2)nc1Nc1ccnc2[nH]ccc12 ; O=C1CCNCC1 ; CC(C)c1cnc(-c2ccc(F)cc2)nc1Nc1ccnc2[nH]cc(C3(O)CCNCC3)c12 ; CC(C)c1cnc(-c2ccc(F)cc2)nc1Nc1ccnc2[nH]cc(C3(O)CCNCC3)c12 ; Cl]

### Step 1: Synthesis of 4-(4-((2-(4-fluorophenyl)-5-isopropylpyrimidin-4-yl)amino)-1H-pyrrolo[2,3-b]pyridin-3-yl)piperidin-4-ol

To a solution of N-(2-(4-fluorophenyl)-5-isopropylpyrimidin-4-yl)-1H-pyrrolo[2,3-b]pyridin-4-amine (0.1 g, 0.28 mmol, 1.0 eq.) in 120 ml of ethanol, was added piperidin-4-one (0.38 g, 2.88 mmol, 10.0 eq.) and powdered KOH (0.32 g, 5.7 mmol, 20.0 eq.). Reaction mixture was heated at 130° C. for 48 hours followed by monitoring with LCMS. Solvent was evaporated and residue was pre-cleaned by combi-flash. Then the crude material was purified by PREP-HPLC to give 11 mg of 4-(4-((2-(4-fluorophenyl)-5-isopropylpyrimidin-4-yl)amino)-1H-pyrrolo[2,3-b]pyridin-3-yl)piperidin-4-ol. LCMS (M+1): 447.1 ¹H NMR (400 MHz, DMSO-d₆): δ 11.60 (br. s., 1H), 10.59 (s, 1H), 8.56 (s, 1H), 8.39 (dd, J=5.87, 9.00 Hz, 2H), 8.22 (s, 2H), 7.32-7.38 (m, 3H), 6.99 (s, 1H), 3.38-3.45 (m, 1H), 3.20 (m, 4H), 2.12 (m, 4H), 1.34 (d, J=6.65 Hz, 6H).

### Step 2: Synthesis of 4-(4-((2-(4-fluorophenyl)-5-isopropylpyrimidin-4-yl)amino)-1H-pyrrolo[2,3-b]pyridin-3-yl)piperidin-4-ol hydrochloride

To a solution of 1-(4-((2-(4-fluorophenyl)-5-isopropylpyrimidin-4-yl)amino)-1H-pyrrolo[2,3-b]pyridin-3-yl)piperidine-4-carboxamide (0.011 g, 0.024 mmol, 1.0 eq.) in 2 ml of dioxane, was added 2 ml dioxane HCl solution and reaction was stirred for 15 minutes and then solvent was evaporated and this procedure was repeated twice. Solvent was evaporated and residue was lyophilized to get 7 mg 4-(4-((2-(4-fluorophenyl)-5-isopropylpyrimidin-4-yl)amino)-1H-pyrrolo[2,3-b]pyridin-3-yl)piperidin-4-ol hydrochloride (Compound No. 13). LCMS (M+1): 447.3 ¹H NMR (400 MHz, DMSO-d₆): δ 12.53 (s, 1H), 11.30 (s, 1H), 8.74 (m, 3H), 8.36-8.44 (m, 4H), 7.53 (br. s., 2H), 7.37 (t, J=8.80 Hz, 2H), 3.33-3.37 (m, 1H), 3.25 (m, 4H), 2.12-2.21 (m, 4H), 1.35 (d, J=6.65 Hz, 6H).

### Example 14: Preparation of N-(2-(4-fluorophenyl)-5-isopropylpyrimidin-4-yl)-3-(3-methylmorpholino)-1H-pyrrolo [2, 3-b] pyridin-4-amine hydrochloride (Compound No. 14)

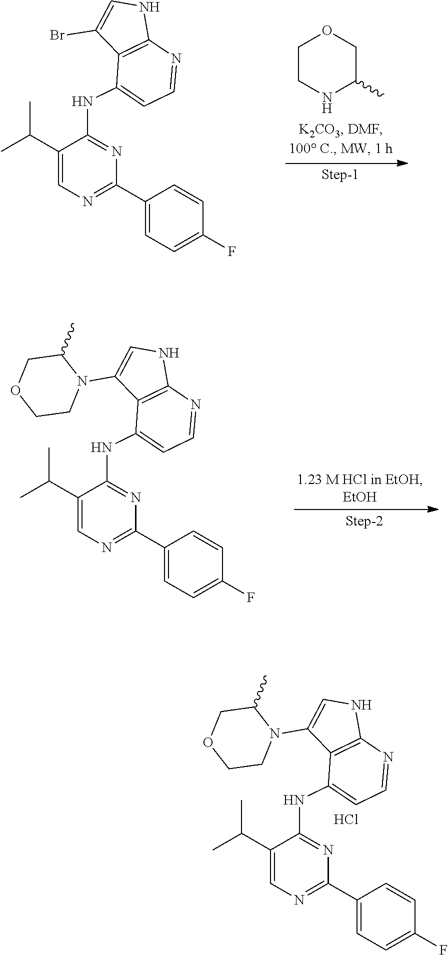

### Step 1: Synthesis of N-(2-(4-fluorophenyl)-5-isopropylpyrimidin-4-yl)-3-(3-methylmorpholino)-1H-pyrrolo [2,3-b] pyridin-4-amine

To a solution of 3-bromo-N-(2-(4-fluorophenyl)-5-isopropylpyrimidin-4-yl)-1H-pyrrolo[2,3-b]pyridin-4-amine (0.100 g, 0.234 mmol, 1.0 eq) in 3 ml of DMF, were added 3-methylmorpholine (0.118 g, 1.17 mmol, 5.0 eq) and K₂CO₃ (0.097 g, 0.702 mmol, 3.0 eq). Reaction mass was heated at 100° C. for 1 hour under microwave. After completion of reaction (LCMS monitoring), reaction mass was filtered to remove K₂CO₃ and filtrate was concentrated on high vacuum pump and crude reaction mixture was purified by PREP-HPLC to give 0.015 g of N-(2-(4-fluorophenyl)-5-isopropylpyrimidin-4-yl)-3-(3-methylmorpholino)-1H-pyrrolo [2, 3-b] pyridin-4-amine as pale yellow solid. LCMS: (M+1): 447.2 ¹H NMR (400 MHz, DMSO-d₆): δ 11.54 (br. s., 1H), 9.95 (s, 1H), 8.61 (s, 1H), 8.38-8.48 (m, 3H), 8.19 (d, J=5.26 Hz, 1H), 7.57 (s, 1H), 7.39 (t, J=8.77 Hz, 2H), 3.93 (d, J=11.40 Hz, 2H), 3.65 (br. s., 1H), 3.42 (br. s., 1H), 3.21-3.29 (m, 1H), 3.07-3.17 (m, 2H), 3.03 (d, J=3.07 Hz, 1H), 1.39 (d, J=12.28 Hz, 3H), 1.41 (d, J=12.28 Hz, 3H), 0.80 (d, J=6.14 Hz, 3H).

### Step 2: Synthesis of N-(2-(4-fluorophenyl)-5-isopropylpyrimidin-4-yl)-3-(3-methylmorpholino)-1H-pyrrolo [2, 3-b] pyridin-4-amine hydrochloride

To the clear solution of N-(2-(4-fluorophenyl)-5-isopropylpyrimidin-4-yl)-3-(3-methylmorpholino)-1H-pyrrolo [2, 3-b] pyridin-4-amine (0.015 g, 0.033 mmol, 1.0 eq.) in 5 ml of ethanol was added 5 ml of 1.23 M solution of HCl in ethanol. Reaction mixture was stirred at it for 10 min and solvent was evaporated under reduced pressure at lower temperature. Similar procedure was repeated twice, residue was submitted for lyophilization to get 0.015 g of N-(2-(4-fluorophenyl)-5-isopropylpyrimidin-4-yl)-3-(3-methylmorpholino)-1H-pyrrolo [2, 3-b]pyridin-4-amine hydrochloride as white solid (Compound No. 14). LCMS (M+1): 447.2; ¹H NMR (400 MHz, DMSO-d₆): δ 12.84 (br. s., 1H), 10.55 (br. s., 1H), 8.82 (s, 1H), 8.71 (d, J=6.65 Hz, 1H), 8.45 (br. s., 3H), 7.85 (br. s., 1H), 7.39 (t, J=8.61 Hz, 2H), 3.95 (d, J=9.00 Hz, 2H), 3.64 (br. s., 1H), 3.43-3.29 (m, 1H), 3.28-3.20 (m, 1H), 3.24-3.16 (m, 2H), 3.15-3.05 (m, 1H), 1.39 (d, J=6.65 Hz, 3H), 1.44 (d, J=6.65 Hz, 3H), 0.82 (d, J=5.87 Hz, 3H).

### Example 15: Preparation of 3-(4-aminopiperidin-1-yl)-N-(2-(4-fluorophenyl)-5-isopropylpyrimidin-4-yl)-1H-pyrrolo[2,3-b]pyridin-4-amine hydrochloride (Compound No. 15)

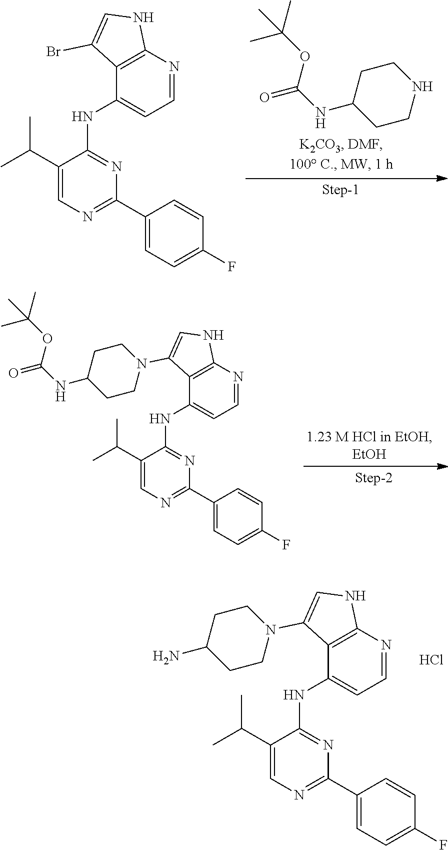 [SMILES: CC(C)c1cnc(-c2ccc(F)cc2)nc1Nc1ccnc2[nH]cc(Br)c12 ; CC(C)(C)OC(=O)NC1CCNCC1 ; CC(C)c1cnc(-c2ccc(F)cc2)nc1Nc1ccnc2[nH]cc(N3CCC(NC(=O)OC(C)(C)C)CC3)c12 ; CC(C)c1cnc(-c2ccc(F)cc2)nc1Nc1ccnc2[nH]cc(N3CCC(N)CC3)c12 ; Cl]

### Step 1: Synthesis of tert-butyl 1-(4-(2-(4-fluorophenyl)-5-isopropylpyrimidin-4-ylamino)-1H-pyrrolo[2,3-b]pyridin-3-yl)piperidin-4-yl carbamate

To a solution of 3-bromo-N-(2-(4-fluorophenyl)-5-isopropylpyrimidin-4-yl)-1H-pyrrolo[2,3-b]pyridin-4-amine (0.100 g, 0.234 mmol, 1.0 eq) in 3 ml of DMF, were added tert-butyl piperidin-4-ylcarbamate (0.234 g, 1.17 mmol, 5.0 eq) and K₂CO₃ (0.064 g, 0.468 mmol, 3.0 eq). Reaction mass was heated at 100° C. for 1 hour under microwave. After completion of reaction (LCMS monitoring), reaction mass was filtered to remove K₂CO₃ and filtrate was concentrated on high vacuum pump and crude reaction mixture was purified by PREP-HPLC to give 0.019 g of tert-butyl 1-(4-(2-(4-fluorophenyl)-5-isopropylpyrimidin-4-ylamino)-1H-pyrrolo[2,3-b]pyridin-3-yl)piperidin-4-ylcarbamate as white solid. ¹H NMR (400 MHz, DMSO-d₆): δ 11.38 (br. s., 1H), 9.89 (br. s., 1H), 8.57 (s, 1H), 8.44 (dd, J=5.92, 8.99 Hz, 2H), 8.37 (d, J=5.70 Hz, 1H), 8.25 (br. s., 1H), 8.16 (d, J=5.70 Hz, 1H), 7.45 (d, J=2.19 Hz, 1H), 7.39 (t, J=8.99 Hz, 2H), 7.09 (br. s., 1H), 3.47 (br. s., 2H), 3.24-3.16 (m, 1H), 2.82-2.91 (m, 2H), 1.85 (br. s., 2H), 1.61 (d, J=10.52 Hz, 3H), 1.35-1.46 (m, 15H).

### Step 2: Synthesis of 3-(4-aminopiperidin-1-yl)-N-(2-(4-fluorophenyl)-5-isopropylpyrimidin-4-yl)-1H-pyrrolo[2,3-b]pyridin-4-amine hydrochloride

To the clear solution of tert-butyl 1-(4-(2-(4-fluorophenyl)-5-isopropylpyrimidin-4-ylamino)-1H-pyrrolo[2,3-b]pyridin-3-yl)piperidin-4-ylcarbamate (0.010 g, 0.018 mmol, 1.0 eq.) in 5 ml of ethanol was added 5 ml of 1.23 M solution of HCl in ethanol. Reaction mixture was stirred at rt for 10 min and solvent was evaporated under reduced pressure at lower temperature. Similar procedure was repeated twice, residue was submitted for lyophilization to get 0.010 g of 3-(4-aminopiperidin-1-yl)-N-(2-(4-fluorophenyl)-5-isopropylpyrimidin-4-yl)-1H-pyrrolo[2,3-b]pyridin-4-amine hydrochloride as white solid (Compound No. 15). LCMS: (M+1)=446.1 ¹H NMR (400 MHz, DMSO-d₆): δ 12.76 (br. s., 1H), 10.51 (s, 1H), 8.75 (br. s., 1H), 8.65 (br. s., 1H), 8.39 (s, 3H), 8.43 (s, 3H), 7.83 (br. s., 1H), 7.39 (t, J=8.41 Hz, 2H), 3.32-3.24 (m, 2H), 3.18-3.12 (m, 2H), 3.16-2.92 (m, 2H), 1.98-1.88 (m, 2H), 1.88-1.78 (m, 2H), 1.43 (d, J=6.65 Hz, 6H).

### Example 16: Preparation of 2-(4-((2-(4-fluorophenyl)-5-isopropylpyrimidin-4-yl) amino)-1H-pyrrolo[2,3-b]pyridin-3-yl)-N,N-dimethyl-2-oxoacetamide (Compound No. 16)

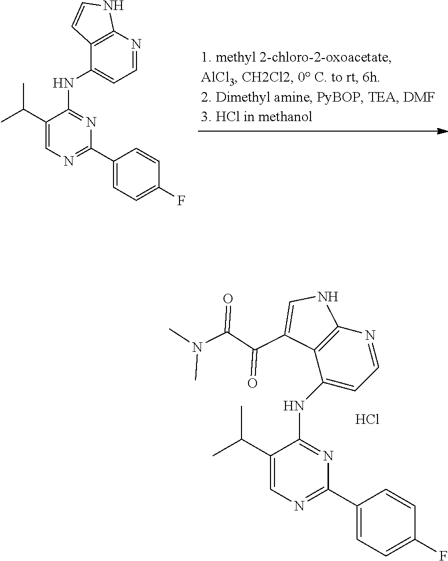 [SMILES: CC(C)c1cnc(-c2ccc(F)cc2)nc1Nc1ccnc2[nH]ccc12 ; CC(C)c1cnc(-c2ccc(F)cc2)nc1Nc1ccnc2[nH]cc(C(=O)C(=O)N(C)C)c12 ; Cl]

To the solution of N-(2-(4-fluorophenyl)-5-isopropylpyrimidin-4-yl)-1H-pyrrolo[2,3-b]pyridin-4-amine (0.15 g, 0.43 mmol, 1.0 eq.) in 30 ml CH₂Cl₂ cooled at 0° C. were added aluminum trichloride (0.288 g, 2.15 mmol, 5.0 eq.) and methyl 2-chloro-2-oxoacetate (0.389 g, 3.58 mmol, 8.6 eq. (d=1.33 g/ml)) under N₂ atmosphere. Reaction mass was allowed to warm at room temperature and then stirred for 48 h. After completion reaction (LCMS monitoring) methanol (30 ml) was added to reaction mass and again it was stirred at overnight. Then methanol was removed by distillation and crude was taken in 100 ml of ethyl acetate. Organic layer was washed by water twice (2×100 ml) and brine solution twice (2×100 ml). Dried over anhydrous Na2SO4, and solvent was evaporated under reduced pressure at low temperature to obtain 0.250 g of crude material. Crude material was dissolved in THF and its pH was adjusted to 9-10 using 2M aq. NaOH and reaction was stirred for 18 h to get corresponding acid (confirmed by LCMS). Reaction mixture was acidified to pH 1 using aq. HCl and then was extracted with ethyl acetate thrice; the organic layer was evaporated to get 0.2 g of crude acid (confirmed by LCMS). The acid (crude 0.08 g, 0.19 mmol, 1 eq.) was dissolved in DMF (5 ml) add to it PyBop (0.129 g, 0.24 mmol, 1.3 eq.), triethyl amine (0.05 g, 0.49 mmol, 2.6 eq.) and 2M dimethyl amine solution in THF (10 ml) and reaction was stirred at room temperature for 24 h followed by LCMS. Solvent was evaporated and residue was diluted with water and then extracted with ethylacetae three times and separated organic layer was evaporated to get crude product. The crude product was purified by PREP-HPLC. The product was then treated with 4 M dioxane in HCl and solvent was evaporated. The residue was lyophilized to get 12 mg of 2-(4-((2-(4-fluorophenyl)-5-isopropylpyrimidin-4-yl) amino)-1H-pyrrolo[2,3-b]pyridin-3-yl)-N,N-dimethyl-2-oxoacetamide. LCMS (M+1): 447.2; ¹H-NMR: (400 MHz, DMSO-d6) δ 11.22 (s, 1H), 8.77 (d, J=5.70 Hz, 1H), 8.60 (s, 1H), 8.38-8.47 (m, 3H), 8.37 (s, 1H), 7.40 (t, J=8.77 Hz, 2H), 3.56-3.63 (m, 1H), 2.99-3.03 (m, 6H), 1.32-1.41 (m, 6H); ¹H NMR D₂O (400 MHz, DMSO-d6) δ 8.76 (d, J=6.14 Hz, 1H), 8.53 (s, 1H), 8.33-8.42 (m, 3H), 8.29 (s, 1H), 7.37 (t, J=8.99 Hz, 2H), 3.50-3.55 (m, 1H), 1.33 (d, J=7.02 Hz, 6H).

### Example 17: Preparation of 2-(4-((2-(4-fluorophenyl)-5-isopropylpyrimidin-4-yl)amino)-1H-pyrrolo[2,3-b]pyridin-3-yl)-2-oxoacetamide hydrochloride (Compound No. 17)

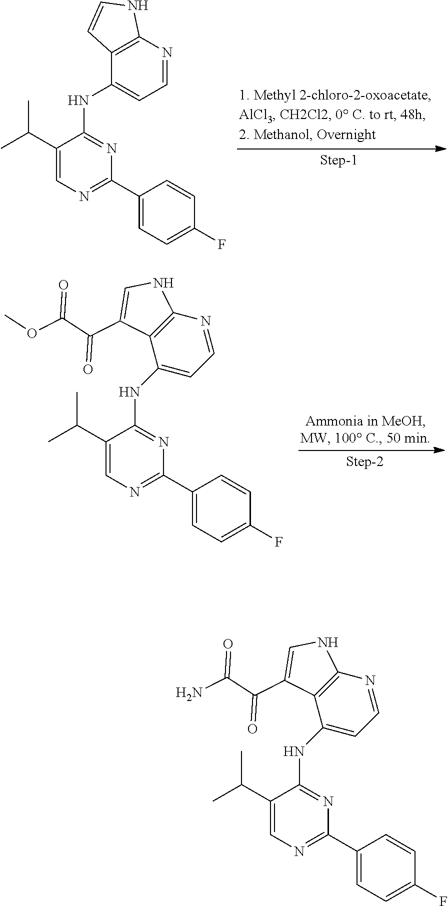

### Step-1: Synthesis of methyl 2-(4-((2-(4-fluorophenyl)-5-isopropylpyrimidin-4-yl)amino)-1H-pyrrolo[2,3-b]pyridin-3-yl)-2-oxoacetate

To the solution of N-(2-(4-fluorophenyl)-5-isopropylpyrimidin-4-yl)-1H-pyrrolo[2,3-b]pyridin-4-amine (0.6 g, 1.72 mmol, 1.0 eq.) in CH₂Cl₂ (100 ml) cooled at 0° C. were added aluminum trichloride (1.14 g, 8.6 mmol, 5.0 eq.) and methyl 2-chloro-2-oxoacetate (3.11 g, 28.6 mmol, 16.6 eq. (d=1.33 g/ml) under N₂ atmosphere. Reaction mass was allowed to warm at room temperature and then stirred for 48 h. After completion reaction (LCMS monitoring) methanol (30 ml) was added to reaction mass and again it was stirred at overnight. Then methanol was removed by distillation and crude was taken in 100 ml of ethyl acetate. Organic layer was washed by water twice (2×100 ml) and brine solution twice (2×100 ml). Dried over anhydrous Na₂SO₄, and solvent was evaporated under reduced pressure at low temperature to obtain 1.2 g of crude material. The crude compound was purified by combi-flash chromatography using 0-5% MeOH in dichloroform get 0.4 g of methyl 2-(4-((2-(4-fluorophenyl)-5-isopropylpyrimidin-4-yl)amino)-1H-pyrrolo[2,3-b]pyridin-3-yl)-2-oxoacetate. LCMS (M+1): 434.2; ¹H NMR (400 MHz, DMSO-d6) δ 11.09 (s, 1H), 8.67-8.75 (m, 2H), 8.60 (s, 1H), 8.36-8.45 (m, 3H), 7.40 (t, J=8.77 Hz, 3H), 3.96 (s, 3H), 3.50-3.56 (m, 1H), 1.37 (d, J=6.58 Hz, 6H); ¹H NMR D20 (400 MHz, DMSO-d6) δ 8.69 (d, J=6.14 Hz, 1H), 8.63 (s, 1H), 8.53 (s, 1H), 8.41 (d, J=6.14 Hz, 1H), 8.35 (dd, J=5.48, 8.55 Hz, 2H), 7.37 (t, J=8.77 Hz, 2H), 3.93 (s, 3H), 3.46-3.53 (m, 1H), 1.34 (d, J=7.02 Hz, 6H).

### Step-2: Synthesis of 2-(4-((2-(4-fluorophenyl)-5-isopropylpyrimidin-4-yl)amino)-1H-pyrrolo[2,3-b]pyridin-3-yl)-2-oxoacetamide hydrochloride

To the solution of methyl 2-(4-((2-(4-fluorophenyl)-5-isopropylpyrimidin-4-yl)amino)-1H-pyrrolo[2,3-b]pyridin-3-yl)-2-oxoacetate (0.08 g, 0.18 mmol, 1.0 eq.) in methanol (0.2 ml) was added ammonia in methanol (6 M, 2 ml) and reaction was heated in microwave at 100° C. for 50 min, keeping cooling temperature 40° C. After first run solvent was evaporated and above procedure was repeated twice. Solvent was evaporated and residue was purified by RP-HPLC to get 28 mg of 2-(4-((2-(4-fluorophenyl)-5-isopropylpyrimidin-4-yl)amino)-1H-pyrrolo[2,3-b]pyridin-3-yl)-2-oxoacetamide. To the solution of 2-(4-((2-(4-fluorophenyl)-5-isopropylpyrimidin-4-yl)amino)-1H-pyrrolo[2,3-b]pyridin-3-yl)-2-oxoacetamide (0.027 g, 0.064 mmol, 1.0 eq.) in ethanol (2 ml) was added HCl in methanol (2 ml) and reaction was stirred at room temperature for 2 h. Solvent was evaporated and residue was lyophilized to get 14 mg of 2-(4-((2-(4-fluorophenyl)-5-isopropylpyrimidin-4-yl)amino)-1H-pyrrolo[2,3-b]pyridin-3-yl)-2-oxoacetamide hydrochloride. LCMS (M+1): 419.3; ¹H-NMR: (400 MHz, DMSO-d6) δ 11.26 (s, 1H), 8.65-8.72 (m, 2H), 8.60 (s, 1H), 8.42 (dd, J=5.92, 8.55 Hz, 2H), 8.36 (d, J=5.70 Hz, 1H), 8.23 (br. s., 1H), 7.95 (br. s., 1H), 7.37 (t, J=8.77 Hz, 2H), 3.56-3.62 (m, 1H), 1.38 (d, J=6.58 Hz, 6H); ¹H NMR D20: (400 MHz, DMSO-d6) δ 8.69 (dd, J=3.51, 5.70 Hz, 1H), 8.62 (s, 1H), 8.52 (s, 1H), 8.30-8.42 (m, 3H), 7.33 (t, J=8.77 Hz, 2H), 3.51-3.56 (m, 1H), 1.33 (d, J=6.58 Hz, 6H).

### Example 18: Preparation of Synthesis of 2-(4-((2-(5-chloro-2-fluorophenyl)-5-isopropylpyrimidin-4-yl)amino)-1H-pyrrolo[2,3-b]pyridin-3-yl)-N-methyl-2-oxoacetamide hydrochloride (Compound No. 18)

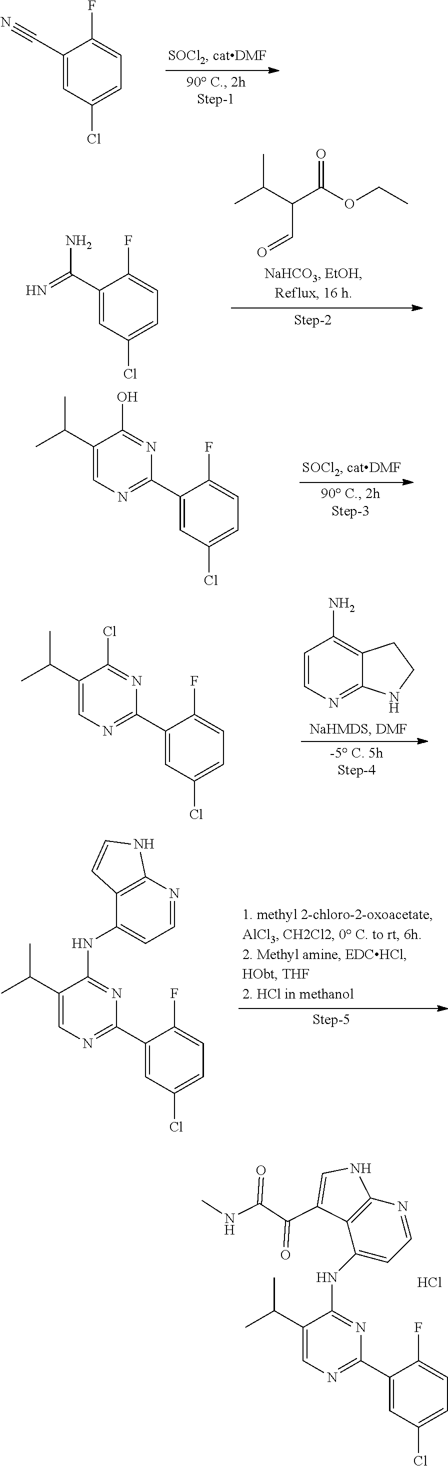

### Step-1 and Step-2: Synthesis of 2-(5-chloro-2-fluorophenyl)-5-isopropylpyrimidin-4-ol

To the solution of 5-chloro-2-fluorobenzonitrile (5.0 g, 32.0 mmol, 1.0 eq.) in CH₂Cl₂ (500 ml) at 0° C. NaHDMS (17.68 g, 96.0 mmol, 3 eq.) was added drop wise. The reaction was stirred at 0° C. for 30 min and then it was warmed to room temperature and then stirring was continued for 2 h. Solvent was evaporated and residue was washed with water and then extracted with ethyl acetate thrice. Combined organic layer was washed with 1 N HCl, separated organic layer was evaporated to get solid compound. The material was solubilized in ethanol (600 ml) and ethyl 2-formyl-3-methylbutanoate (5.07 g, 32.0 mmol, 1 eq.), NaHCO₃ (8.09 g, 96.3 mmol, 3.0 eq) was added to it, reaction mixture was heated to reflux for 4 h. The progress of reaction was monitored by TLC and LCMS. Upon completion of the reaction ethanol was removed under reduced pressure to get crude product. Crude material was triturated with n-pentane to give 5.0 g of 2-(5-chloro-2-fluorophenyl)-5-isopropylpyrimidin-4-ol. The product was confirmed by 1H-NMR and LCMS. LCMS (M+1): 267.1; ¹H-NMR: (400 MHz, CHLOROFORM-d) δ 8.13 (dd, J=2.63, 6.58 Hz, 1H), 7.93 (s, 1H), 7.47 (ddd, J=2.85, 4.17, 8.77 Hz, 1H), 7.16 (dd, J=8.77, 10.96 Hz, 1H), 3.03-3.14 (m, 1H), 1.26 (d, J=7.02 Hz, 6H).

### Step-3: Synthesis of 4-chloro-2-(5-chloro-2-fluorophenyl)-5-isopropylpyrimidine

To the suspension of 2-(5-chloro-2-fluorophenyl)-5-isopropylpyrimidin-4-ol (3.0 g, 11.2 mmol, 1 eq.) in 15 ml thionyl chloride taken in 50 ml sealed tube was added 2-3 drops of DMF. Reaction mass was heated at 85° C. for 1.5 h. Progress of reaction was monitored by TLC. Upon completion of reaction thionyl chloride was removed under reduced pressure. Residue was dissolved in ethyl acetate 150 ml, and then organic layer was washed with 10% NaHCO₃ twice to remove remaining thionyl chloride followed by water and brine solution. Dried over anhydrous Na₂SO₄, and solvent was evaporated under reduced pressure at low temperature to obtain 3 g of 4-chloro-2-(5-chloro-2-fluorophenyl)-5-isopropylpyrimidine as white solid. Product was confirmed by 1H-NMR. LCMS (M+1): 285.0; ¹H-NMR: (400 MHz, CHLOROFORM-d) δ 8.70 (s, 1H), 8.08 (dd, J=2.63, 6.58 Hz, 1H), 7.40 (td, J=3.34, 8.66 Hz, 1H), 7.10-7.18 (m, 1H), 3.29-3.41 (m, 1H), 1.37 (d, J=7.02 Hz, 6H).

### Step-4: Synthesis of N-(2-(5-chloro-2-fluorophenyl)-5-isopropylpyrimidin-4-yl)-1H-pyrrolo[2,3-b]pyridin-4-amine

To the solution of compound 4-amino 7-azaindole (0.5 g, 3.75 mmol, 1.0 eq.) in 5 ml DMF cooled at −0° C. were added 4-chloro-2-(5-chloro-2-fluorophenyl)-5-isopropylpyrimidine (1.07 g, 3.75 mmol, 1.0 eq.) followed by drop wise addition of NaHMDS solution (4.13 g (13.05 ml), 22.5 mmol, 35% in THF, 6 eq.) in two lots, under N₂ atm. Reaction mass was continued at same temperature for 5 h. After completion of reaction (TLC monitoring), reaction mass was poured in 250 g of ice cooled water. Resulting solid was filtered out to get 1.4 g of crude product. Crude material was purified by silica gel column chromatography (100-200) mesh silica with 0-3% MeOH:DCM to obtain 1.2 g of N-(2-(5-chloro-2-fluorophenyl)-5-isopropylpyrimidin-4-yl)-1H-pyrrolo[2,3-b]pyridin-4-amine, product was characterized by ¹H-NMR and LCMS. LCMS (M+1): 382.2; ¹H-NMR: (400 MHz, DMSO-d6) δ 11.56 (br. s., 1H), 8.81 (br. s., 1H), 8.51 (s, 1H), 8.12 (d, J=5.26 Hz, 1H), 7.88 (dd, J=2.85, 6.36 Hz, 1H), 7.45-7.59 (m, 2H), 7.24-7.42 (m, 2H), 6.47 (d, J=3.51 Hz, 1H), 3.37-3.53 (m, 1H), 1.34 (d, J=6.58 Hz, 6H).

### Step-5: Synthesis of 2-(4-((2-(5-chloro-2-fluorophenyl)-5-isopropylpyrimidin-4-yl)amino)-1H-pyrrolo[2,3-b]pyridin-3-yl)-N-methyl-2-oxoacetamide hydrochloride

To the solution of N-(2-(5-chloro-2-fluorophenyl)-5-isopropylpyrimidin-4-yl)-1H-pyrrolo[2,3-b]pyridin-4-amine (0.7 g, 1.8 mmol, 1.0 eq.) in CH₂Cl₂ (50 ml) cooled at 0° C. were added aluminum trichloride (1.22 g, 9.1 mmol, 5.0 eq.) and methyl 2-chloro-2-oxoacetate (3.3 g, 30.0 mmol, 16.6 eq. (d=1.33 g/ml)) under N₂ atmosphere. Reaction mass was allowed to warm at room temperature and then stirred for 48 h. After completion reaction (LCMS monitoring) methanol (30 ml) was added to reaction mass and again it was stirred at overnight. Then methanol was removed and reaction mixture was stirred in THF (50 ml) and 2 M NaOH solution for 12 h, then solvent was evaporated and residue was acidified using 2 M HCl again extracted with ethyl acetate thrice. Organic layer was dried over anhydrous Na₂SO₄, and solvent was evaporated under reduced pressure at low temperature to obtain 0.15 g of crude material of methyl 2-(4-((2-(5-chloro-2-fluorophenyl)-5-isopropylpyrimidin-4-yl)amino)-1H-pyrrolo[2,3-b]pyridin-3-yl)-2-oxoacetate. To the solution of methyl methyl 2-(4-((2-(5-chloro-2-fluorophenyl)-5-isopropylpyrimidin-4-yl)amino)-1H-pyrrolo[2,3-b]pyridin-3-yl)-2-oxoacetate (0.1 g, 2.0 mmol, 1.0 eq.) in methanol (0.8 ml) was added ammonia in methanol (6 M, 2 ml) and reaction was heated in microwave at 100° C. for 50 min, keeping cooling temperature 40° C. After first run solvent was evaporated and above procedure was repeated twice. Solvent was evaporated and residue was purified by RP-HPLC. Purified compound was treated with 4 M dioxane in HCl (3×2 ml), solvent was evaporated and residue was lyophilized to get 18 mg of 2-(4-((2-(5-chloro-2-fluorophenyl)-5-isopropylpyrimidin-4-yl)amino)-1H-pyrrolo[2,3-b]pyridin-3-yl)-N-methyl-2-oxoacetamide hydrochloride. LCMS (M+1): 467.2; ¹H-NMR: (400 MHz, DMSO-d6) δ 11.32 (s, 1H), 8.73-8.81 (m, 3H), 8.65 (s, 1H), 8.25 (d, J=5.70 Hz, 1H), 8.08 (dd, J=2.63, 6.58 Hz, 1H), 7.62 (br. s., 2H), 6.52 (s, 2H), 3.58-3.66 (m, 1H), 2.81 (d, J=4.82 Hz, 3H), 1.39 (d, J=7.02 Hz, 6H).

### Example 19: Preparation of 2-(4-((2-(4-fluorophenyl)-5-isopropylpyridin-4-yl)amino)-1H-pyrrolo[2,3-b]pyridin-3-yl)-2-oxoacetamide hydrochloride (Compound No. 19)

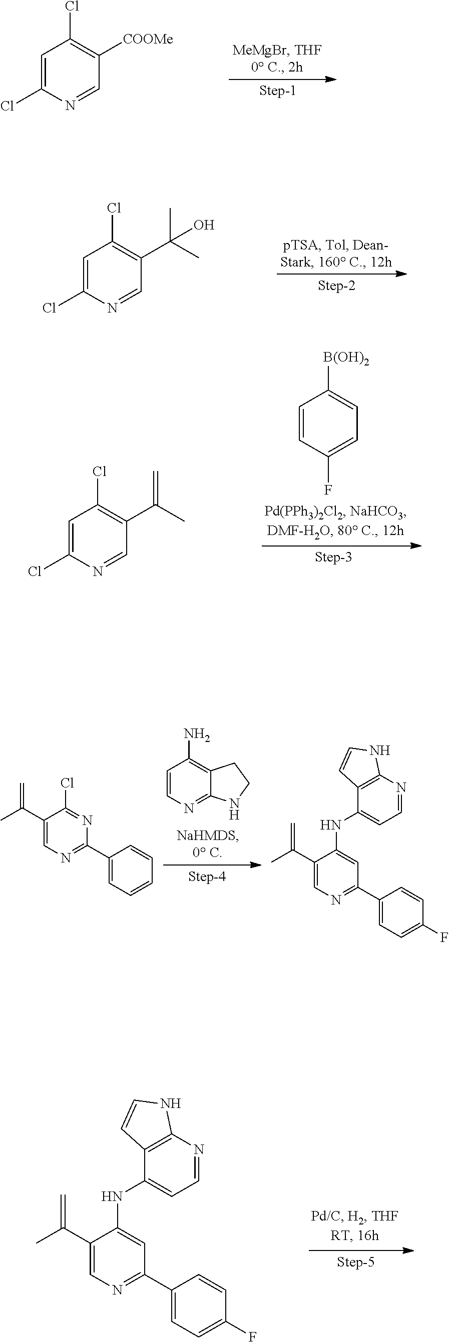

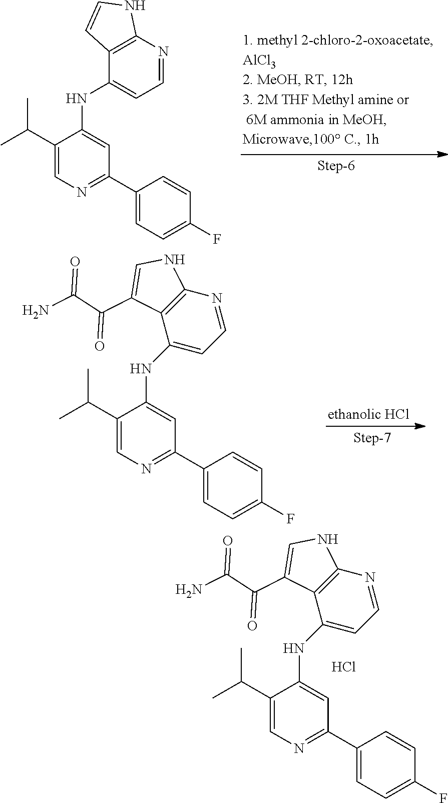

### Step-1: Synthesis of 2-(4,6-dichloropyridin-3-yl)propan-2-ol

To solution of methyl 4,6-dichloronicotinate (4.5 g, 22 mmol, 1.0 eq) in 40 ml of THF cooled at 0° C. was added solution of MeMgBr (25.4 ml, 76 mmol, 3.5 eq, 3 M in ether) under nitrogen atmosphere. Reaction mass was stirred at same temperature for 4 h. After completion of reaction (TLC monitoring) excess MeMgBr was quenched by saturated NH₄Cl solution. Desired product was extracted from aq. in ethyl acetate twice (2×100 ml). Organic layer was washed with brine (2×50 ml) and water (2×50 ml). Dried over anhydrous Na₂SO₄, and solvent was evaporated to obtain 4.5 g 2-(4,6-dichloropyridin-3-yl)propan-2-ol as pale yellow compound. LCMS (M+1): 208; 1H-NMR: (400 MHz, CHCl3-d3) δ 8.8 (s, 1H), 7.38 (s, 1H), 1.74 (s, 6H).

### Step-2: Synthesis of 2,4-dichloro-5-(prop-1-en-2-yl)pyridine

To solution of 2-(4,6-dichloropyridin-3-yl)propan-2-ol (4.5 g, 21 mmol, 1.0 eq) in 100 ml of Toluene was added pTSA (4.1 g, 24 mmol, 1.1 eq) under nitrogen atmosphere. Reaction mass was heated to reflux under dean stark condition to remove water. After completion of reaction toluene was removed by distillation and then crude product was dissolved in ethyl acetate 250 ml. Organic layer was washed with brine (2×50 ml) and water (2×50 ml). Dried over anhydrous Na₂SO₄, and solvent was evaporated and crude was purified by column chromatography (100-200) mesh silica, eluent 5-10% EtOAc:Hexane to obtain 3.6 g 2,4-dichloro-5-(prop-1-en-2-yl)pyridine as pale yellow liquid. ¹H NMR: (400 MHz, CHLOROFORM-d) δ 8.20 (s, 1H), 7.38 (s, 1H), 5.38 (broad s, 1H), 5.06 (broad s, 1H), 2.07 (s, 3H).

### Step-3: Synthesis of 4-chloro-2-(4-fluorophenyl)-5-(prop-1-en-2-yl)pyridine

Solution of (4-fluorophenyl)boronic acid (1.1 g, 8.01 mmol, 1.0 eq), 2,6-dichloro-3-(prop-1-en-2-yl) pyridine (1.5 g, 8.01 mmol, 1.0 eq) and NaHCO₃ (2.0 g, 24.03 mmol, 3.0 eq) in 60 ml of DMF and 15 ml of water was purged with N₂ gas for 15 min. After completion of purging Pd(PPh₃)₂Cl₂ (281 mg, 0.40 mmol, 0.05 eq) was added and reaction mass was again purged with N2 gas for 15 min. Then reaction mass was heated at 80° for 6 h. After completion of reaction, reaction mass was poured in 600 ml of ice water and product was extracted in ethyl acetate. Solvent was evaporated to get 1.6 g of crude 4-chloro-2-(4-fluorophenyl)-5-(prop-1-en-2-yl)pyridine. LCMS (M+1): 248.06

### Step-4: Synthesis of N-(2-(4-fluorophenyl)-5-(prop-1-en-2-yl)pyridin-4-yl)-1H-pyrrolo[2,3-b]pyridin-4-amine

Solution of 4-chloro-2-(4-fluorophenyl)-5-(prop-1-en-2-yl)pyridine (1.2 gm, 4.84 mmol, 1.0 eq), 1H-pyrrolo[2,3-b]pyridin-4-amine (0.646 g, 4.84 mmol, 1.0 eq) and K₃PO₄ (2.0 g, 9.46 mmol, 2.0 eq) in 20 ml of dioxane was purged with N₂ gas for 15 min. After completion of purging Pd₂ (dba)₃ (665 mg, 0.726 mmol, 0.15 eq) and Xanthphos (0.420 g, 0.726 mmol, 0.15 mmol) was added and reaction mass was again purged with N2 gas for 15 min. Then reaction mass was heated at 110° for 48 h. After completion of reaction, reaction mass was poured in 200 ml of ice water and product was extracted in ethyl acetate and solvent was evaporated to obtain crude 1.0 g of N-(2-(4-fluorophenyl)-5-(prop-1-en-2-yl)pyridin-4-yl)-1H-pyrrolo[2,3-b]pyridin-4-amine. LCMS (M+1): 345.14

### Step-5: Synthesis of N-(2-(4-fluorophenyl)-5-isopropylpyridin-4-yl)-1H-pyrrolo[2,3-b]pyridin-4-amine

To solution of crude N-(2-(4-fluorophenyl)-5-(prop-1-en-2-yl)pyridin-4-yl)-1H-pyrrolo[2,3-b]pyridin-4-amine (1.0 g, 2.90 mmol, 1.0 eq) in 100 ml of THF was added Pd/C (300 Mg, 10%). Reaction mass was stirred at rt under H₂ atmosphere for 16 h. After completion of reaction (1H-NMR monitoring), reaction mass was filtered through celite bed and filtrate was concentrated to get 0.9 g of N-(2-(4-fluorophenyl)-5-isopropylpyridin-4-yl)-1H-pyrrolo[2,3-b]pyridin-4-amine as dark solid. Product was confirmed by LCMS. LCMS (M+1): 347.16

### Step-6: Synthesis of 2-(4-((2-(4-fluorophenyl)-5-isopropylpyridin-4-yl) amino)-1H-pyrrolo [2,3-b]pyridin-3-yl)-2-oxoacetamide

To the solution of N-(2-(4-fluorophenyl)-5-isopropylpyridin-4-yl)-1H-pyrrolo[2,3-b]pyridin-4-amine (0.7 g, 1.8 mmol, 1.0 eq.) in 300 ml CH₂Cl₂ cooled at 0° C. was added aluminum trichloride (1.21 g, 9.1 mmol, 5.0 eq.) and methyl 2-chloro-2-oxoacetate (1.98 g, 18.0 mmol, 10 eq.) under N₂ atmosphere. Reaction mass was allowed to warm at room temperature and then stirred for 48 h. After completion reaction (LCMS monitoring) methanol (50 ml) was added to reaction mass and again it was stirred for overnight. Then methanol was removed by distillation and crude was taken in 250 ml of ethyl acetate. Organic layer was washed by water twice (2×100 ml) and brine solution twice (2×100 ml). Dried over anhydrous Na₂SO₄, and solvent was evaporated under reduced pressure. Crude product was purified by flash column chromatography to give 0.3 g of methyl 2-(4-((2-(4-fluorophenyl)-5-isopropylpyridin-4-yl)amino)-1H-pyrrolo[2,3-b]pyridin-3-yl)-2-oxoacetate. Solution of methyl 2-(4-((2-(4-fluorophenyl)-5-isopropylpyridin-4-yl)amino)-1H-pyrrolo[2,3-b]pyridin-3-yl)-2-oxoacetate (0.1 g, 0.231 mmol, 1.0 eq) in 15 ml of methanolic ammonia was heated at 100° C. for 1 h in microwave. After completion of reaction methanol was removed by distillation and crude was purified by PREP-HPLC to give 26 mg of 2-(4-((2-(4-fluorophenyl)-5-isopropylpyridin-4-yl) amino)-1H-pyrrolo [2,3-b]pyridin-3-yl)-2-oxoacetamide. LCMS (M+1): 418.2; ¹H NMR: (400 MHz, DMSO-d6) δ 10.38 (broad s, 1H), 8.61 (d, J=3.07 Hz, 2H), 8.21 (broad s, 1H), 8.05-8.13 (m, 3H), 7.89-7.93 (m, 2H), 7.29 (t, J=8.77 Hz, 2H), 7.03 (d, J=6.14 Hz, 1H), 3.37-3.43 (m, 1H), 1.37 (d, J=7.02 Hz, 6H).

### Step-7; Synthesis of 2-(4-((2-(4-fluorophenyl)-5-isopropylpyridin-4-yl)amino)-1H-pyrrolo[2,3-b]pyridin-3-yl)-2-oxoacetamide hydrochloride

To the clear solution of 2-(4-((2-(4-fluorophenyl)-5-isopropylpyridin-4-yl)amino)-1H-pyrrolo[2,3-b]pyridin-3-yl)-2-oxoacetamide (0.024 g, 0.057 mmol, 1.0 eq) in 8 ml of methanol was added 5 ml of solution of HCl in methanol. Solution was stirred at rt for 10 min then solvent was evaporated under reduced pressure at lower temperature. Similar procedure was repeated for 2 more times and then compound was submitted for lypholisation to get 22 mg of 2-(4-((2-(4-fluorophenyl)-5-isopropylpyridin-4-yl)amino)-1H-pyrrolo[2,3-b]pyridin-3-yl)-2-oxoacetamide hydrochloride. LCMS (M+1): 418.2; ¹H-NMR: (400 MHz, DMSO-d6) δ 11.03 (s, 1H), 8.76 (s, 1H), 8.44 (s, 1H), 8.34 (d, J=5.38 Hz, 1H), 8.21 (br. s., 1H), 7.96-8.04 (m, 4H), 7.43-7.53 (m, 3H), 3.49-3.58 (m, 1H), 1.43 (d, J=6.85 Hz, 6H).

### Example 20: Preparation of N-butyl-2-(4-(2-(4-fluorophenyl)-5-isopropylpyrimidin-4-ylamino)-1H-pyrrolo [2, 3-b] pyridin-3-yl)-2-oxoacetamide hydrochloride (Compound No. 20)

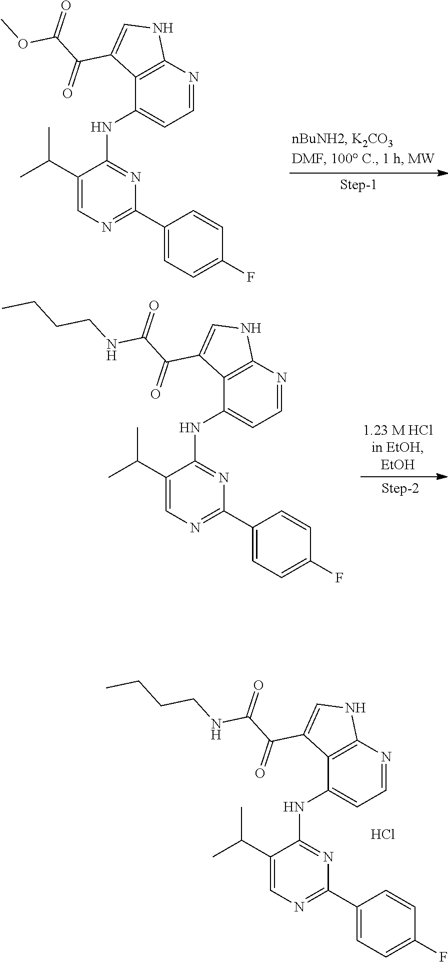

### Step-1; Synthesis of N-butyl-2-(4-(2-(4-fluorophenyl)-5-isopropylpyrimidin-4-ylamino)-1H-pyrrolo [2, 3-b] pyridin-3-yl)-2-oxoacetamide

To solution of methyl 2-(4-(2-(4-fluorophenyl)-5-isopropylpyrimidin-4-ylamino)-1H-pyrrolo [2, 3-b] pyridin-3-yl)-2-oxoacetate (0.150 g, 0.346 mmol, 1.0 eq) in 15 ml of DMF was added n-BuNH₂ (0.45 ml, 4.20 mmol, 5.0 eq). Reaction mass was heated at 100° C. for 1 h under microwave. After completion of reaction (LCMS monitoring), DMF was removed by distillation using high vacuum pump and crude was purified by PREP-HPLC to give 0.025 g of N-butyl-2-(4-(2-(4-fluorophenyl)-5-isopropylpyrimidin-4-ylamino)-1H-pyrrolo [2, 3-b]pyridin-3-yl)-2-oxoacetamide as white solid.

LCMS (M+1): 475.2; ¹H NMR: (400 MHz, DMSO-d₆) δ 13.02 (br. s., 1H), 11.24 (s, 1H), 8.77 (br. s., 1H), 8.64-8.69 (m, 2H), 8.60 (s, 1H), 8.42 (dd, J=5.87, 8.31 Hz, 2H), 8.35 (d, J=5.87 Hz, 1H), 7.37 (t, J=8.80 Hz, 2H), 3.55-3.64 (m, 1H), 3.24-3.28 (m, 2H), 1.49-1.58 (m, 2H), 1.38 (d, J=6.85 Hz, 6H), 1.31-1.36 (m, 2H), 0.92 (t, J=7.34 Hz, 3H).

### Step-2: Synthesis of N-butyl-2-(4-(2-(4-fluorophenyl)-5-isopropylpyrimidin-4-ylamino)-1H-pyrrolo [2, 3-b] pyridin-3-yl)-2-oxoacetamide hydrochloride

To the clear solution of N-butyl-2-(4-(2-(4-fluorophenyl)-5-isopropylpyrimidin-4-ylamino)-1H-pyrrolo [2, 3-b] pyridin-3-yl)-2-oxoacetamide (0.022 g, 0.043 mmol, 1.0 eq) in 10 ml of ethanol was added 5 ml of 1.23 M solution of HCl in ethanol. Solution was stirred at rt for 10 min then solvent was evaporated under reduced pressure at lower temperature. Similar procedure was repeated for 2 more times and then compound was submitted for lyophilisation to get 0.022 g of N-butyl-2-(4-(2-(4-fluorophenyl)-5-isopropylpyrimidin-4-ylamino)-1H-pyrrolo [2, 3-b] pyridin-3-yl)-2-oxoacetamide hydrochloride. LCMS (M+1): 475.2; ¹H NMR: (400 MHz, DMSO-d₆) δ 13.15 (br. s., 1H), 11.36 (s, 1H), 8.82 (t, J=6.11 Hz, 1H), 8.71 (s, 1H), 8.68 (d, J=5.87 Hz, 1H), 8.61 (s, 1H), 8.43 (br. s., 1H), 8.40 (d, J=5.87 Hz, 2H), 7.39 (t, J=8.80 Hz, 2H), 3.54-3.64 (m, 1H), 3.27 (q, J=6.85 Hz, 2H), 1.54 (quin, J=7.21 Hz, 2H), 1.39 (d, J=6.85 Hz, 6H), 1.30-1.36 (m, 2H), 0.92 (t, J=7.34 Hz, 3H).

### Example 21: Preparation of 2-(4-((2-(4-fluorophenyl)-5-isopropylpyridin-4-yl)amino)-1H-pyrrolo[2,3-b]pyridin-3-yl)-N-methyl-2-oxoacetamide hydrochloride (Compound No. 21)

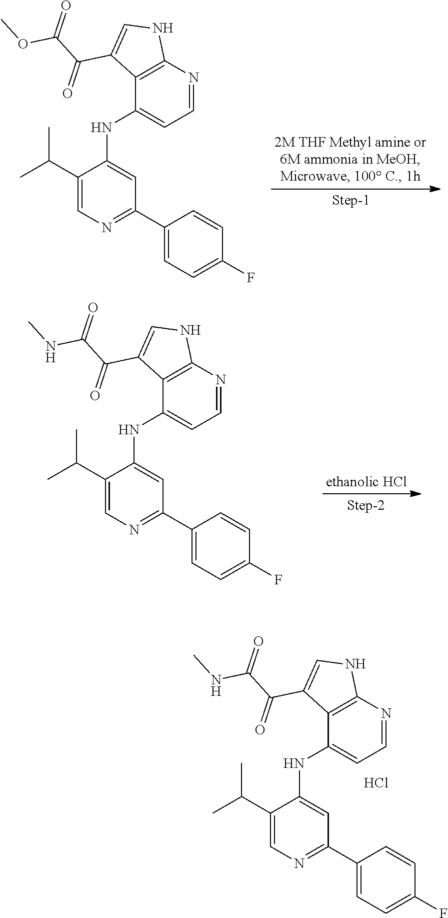 [SMILES: COC(=O)C(=O)c1c[nH]c2nccc(Nc3cc(-c4ccc(F)cc4)ncc3C(C)C)c12 ; CNC(=O)C(=O)c1c[nH]c2nccc(Nc3cc(-c4ccc(F)cc4)ncc3C(C)C)c12 ; CNC(=O)C(=O)c1c[nH]c2nccc(Nc3cc(-c4ccc(F)cc4)ncc3C(C)C)c12 ; Cl]

### Step-1: Synthesis of 2-(4-((2-(4-fluorophenyl)-5-isopropylpyridin-4-yl)amino)-1H-pyrrolo[2,3-b]pyridin-3-yl)-N-methyl-2-oxoacetamide

Crude compound methyl 2-(4-((2-(4-fluorophenyl)-5-isopropylpyridin-4-yl)amino)-1H-pyrrolo[2,3-b]pyridin-3-yl)-2-oxoacetate (0.1 g, 0.231 mmol, 1.0 eq) was taken in 1 ml of methanol and then 5 ml of 2 M methyl amine in THF was added to it. Reaction mixture was heated at 100° C. for 1 h in microwave. After completion of reaction solvent was removed by distillation and crude was purified by PREP-HPLC to give 24 mg of 2-(4-((2-(4-fluorophenyl)-5-isopropylpyridin-4-yl)amino)-1H-pyrrolo[2,3-b]pyridin-3-yl)-N-methyl-2-oxoacetamide. LCMS (M+1): 432.3; ¹H NMR: (400 MHz, DMSO-d6) δ 10.36 (s, 1H), 8.72-8.77 (m, 2H), 8.62 (s, 1H), 8.21 (s, 1H), 8.03-8.12 (m, 3H), 7.93 (s, 1H), 7.29 (t, J=8.77 Hz, 2H), 7.01 (d, J=5.70 Hz, 1H), 3.18-3.26 (m, 1H), 2.77 (d, J=4.82 Hz, 3H), 1.36 (d, J=7.02 Hz, 6H).

### Step-2: Synthesis of 2-(4-((2-(4-fluorophenyl)-5-isopropylpyridin-4-yl)amino)-1H-pyrrolo[2,3-b]pyridin-3-yl)-N-methyl-2-oxoacetamide hydrochloride

To the clear solution of 2-(4-((2-(4-fluorophenyl)-5-isopropylpyridin-4-yl)amino)-1H-pyrrolo[2,3-b]pyridin-3-yl)-N-methyl-2-oxoacetamide (0.024 g, 0.057 mmol, 1.0 eq) in 8 ml of methanol was added 5 ml of solution of HCl in methanol. Solution was stirred at rt for 10 min then solvent was evaporated under reduced pressure at lower temperature. Similar procedure was repeated for 2 more times and then compound was submitted for lypholisation to get 18 mg of 2-(4-((2-(4-fluorophenyl)-5-isopropylpyridin-4-yl)amino)-1H-pyrrolo[2,3-b]pyridin-3-yl)-N-methyl-2-oxoacetamide hydrochloride. LCMS (M+1): 432.2; ¹H NMR: (400 MHz, DMSO-d6) δ 10.94 (br. s., 1H), 8.84 (s, 1H), 8.77 (br. s., 1H), 8.45 (s, 1H), 8.32 (d, J=5.38 Hz, 1H), 7.99-8.05 (m, 2H), 7.97 (s, 1H), 7.38-7.50 (m, 3H), 3.58-3.47 (m, 1H), 2.78 (d, J=4.89 Hz, 3H), 1.43 (d, J=6.85 Hz, 6H).

### Example 22: Preparation of 1-(4-(2-(4-fluorophenyl)-5-isopropylpyrimidin-4-ylamino)-1H-pyrrolo [2, 3-b] pyridin-3-yl)-2-morpholinoethane-1, 2-dione hydrochloride (Compound No. 22)

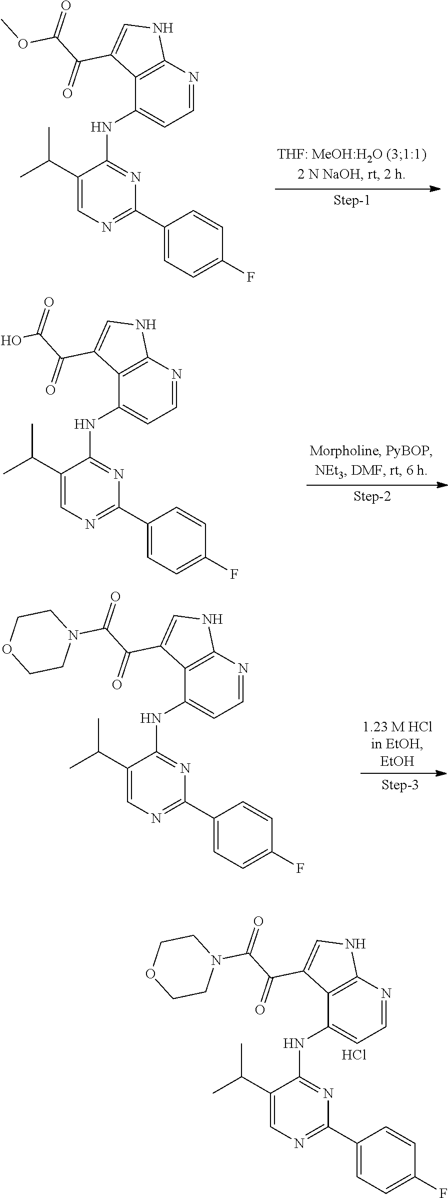

### Step-1: Synthesis of 2-(4-(2-(4-fluorophenyl)-5-isopropylpyrimidin-4-ylamino)-1H-pyrrolo [2,3-b] pyridin-3-yl)-2-oxoacetic acid

To a solution of methyl 2-(4-(2-(4-fluorophenyl)-5-isopropylpyrimidin-4-ylamino)-1H-pyrrolo [2, 3-b] pyridin-3-yl)-2-oxoacetate (1.5 g, 3.46 mmol, 1.0 eq) in 100 ml system of THF:MeOH:H2O (3:1:1) was slowly added 15 ml of 2 M aq.NaOH solution at 0° C. Reaction mass was allowed to warm at rt and then stirred for 2 h. After completion of reaction, organic solvent was removed by distillation. Remaining aq, was extracted with ethyl acetate three times (3×100 ml) to remove starting material and other impurities. Aq layer was acidify by 2 N HCl solution, resulting solid was filtered to get 0.9 g of 2-(4-(2-(4-fluorophenyl)-5-isopropylpyrimidin-4-ylamino)-1H-pyrrolo [2,3-b] pyridin-3-yl)-2-oxoacetic acid as white solid. LCMS (M+1): 420.1

### Step-2: Synthesis of 1-(4-(2-(4-fluorophenyl)-5-isopropylpyrimidin-4-ylamino)-1H-pyrrolo [2, 3-b] pyridin-3-yl)-2-morpholinoethane-1, 2-dione

To solution of 2-(4-(2-(4-fluorophenyl)-5-isopropylpyrimidin-4-ylamino)-1H-pyrrolo [2, 3-b] pyridin-3-yl)-2-oxoacetic (0.100 g, 0.238 mmol, 1.0 eq) in 15 ml of DMF were added NEt₃ (0.1 ml, 0.715 mmol, 3.0 eq) and PyBOP (0.160 g, 0.309 mmol, 1.3 eq) at rt. Reaction mass was allowed to stirred at rt for 15 min and then morpholine (0.04 ml, 0.476 mmol, 2.0 eq) was added slowly drop wise. Reaction was stirred for 6 h under N₂ atm. After completion of reaction DMF was removed by high vacuum distillation and product was purified by PREP-HPLC to get 0.019 g of 1-(4-(2-(4-fluorophenyl)-5-isopropylpyrimidin-4-ylamino)-1H-pyrrolo [2, 3-b] pyridin-3-yl)-2-morpholinoethane-1, 2-dione as white solid. LCMS (M+1): 489.1; ¹H NMR: (400 MHz, DMSO-d₆) δ 13.19 (br. s., 1H), 11.04 (s, 1H), 8.77 (d, J=5.87 Hz, 1H), 8.60 (s, 1H), 8.46 (s, 1H), 8.42 (dd, J=6.60, 9.54 Hz, 2H), 8.39 (s, 1H), 7.38 (t, J=8.80 Hz, 2H), 3.74 (d, J=4.89 Hz, 2H), 3.66 (d, J=4.89 Hz, 2H), 3.58 (br. s., 1H), 3.54 (d, J=4.89 Hz, 2H), 3.42 (d, J=4.40 Hz, 2H), 1.36 (d, J=6.36 Hz, 6H).

### Step-3: Synthesis of 1-(4-(2-(4-fluorophenyl)-5-isopropylpyrimidin-4-ylamino)-1H-pyrrolo [2, 3-b] pyridin-3-yl)-2-morpholinoethane-1, 2-dione hydrochloride

To the clear solution of 1-(4-(2-(4-fluorophenyl)-5-isopropylpyrimidin-4-ylamino)-1H-pyrrolo [2, 3-b] pyridin-3-yl)-2-morpholinoethane-1, 2-dione (0.019 g, 0.038 mmol, 1.0 eq) in 10 ml of ethanol was added 5 ml of 1.23 M solution of HCl in ethanol. Solution was stirred at rt for 10 min then solvent was evaporated under reduced pressure at lower temperature. Similar procedure was repeated for 2 more times and then compound was submitted for lyophilisation to get 0.019 g of 1-(4-(2-(4-fluorophenyl)-5-isopropylpyrimidin-4-ylamino)-1H-pyrrolo [2, 3-b] pyridin-3-yl)-2-morpholinoethane-1, 2-dione hydrochloride. LCMS (M+1): 489.1; ¹H NMR: (400 MHz, DMSO-d₆) δ 13.31 (br. s., 1H), 11.15 (s, 1H), 8.75 (d, J=5.70 Hz, 1H), 8.60 (s, 1H), 8.48 (s, 1H), 8.41-8.45 (m, 2H), 8.38-8.41 (m, 1H), 3.74 (d, J=4.82 Hz, 3H), 3.52-3.55 (m, 3H), 3.42 (d, J=4.38 Hz, 2H), 1.36 (d, J=7.02 Hz, 6H).

### Example 23: Preparation of 1-(4-(2-(4-fluorophenyl)-5-isopropylpyrimidin-4-ylamino)-1H-pyrrolo [2, 3-b] pyridin-3-yl)-2-(piperazin-1-yl) ethane-1,2-dione hydrochloride (Compound No. 23)

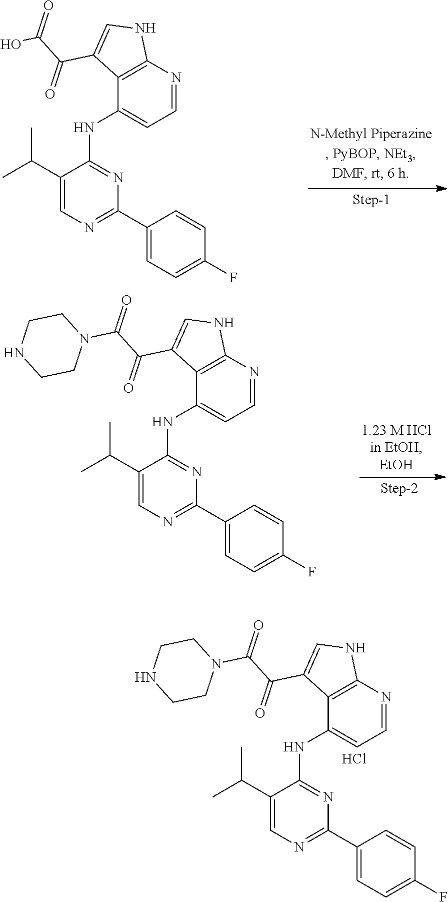

### Step-1: Synthesis of 1-(4-(2-(4-fluorophenyl)-5-isopropylpyrimidin-4-ylamino)-1H-pyrrolo [2, 3-b] pyridin-3-yl)-2-(piperazin-1-yl) ethane-1, 2-dione

To solution of 2-(4-(2-(4-fluorophenyl)-5-isopropylpyrimidin-4-ylamino)-1H-pyrrolo [2, 3-b]pyridin-3-yl)-2-oxoacetic (0.100 g, 0.238 mmol, 1.0 eq) in 15 ml of DMF were added NEt₃ (0.1 ml, 0.714 mmol, 3.0 eq) and PyBOP (0.161 g, 0.309 mmol, 1.3 eq) at rt. Reaction mass was allowed to stirred at rt for 15 min and then piperazine (0.040 g, 0.476 mmol, 2.0 eq) was added slowly drop wise. Reaction was stirred for 6 h under N₂ atm. After completion of reaction DMF was removed by high vacuum distillation and product was purified by PREP-HPLC to get 0.030 g of 1-(4-(2-(4-fluorophenyl)-5-isopropylpyrimidin-4-ylamino)-1H-pyrrolo [2, 3-b] pyridin-3-yl)-2-(piperazin-1-yl) ethane-1, 2-dione as white solid. LCMS (M+1): 488.2; ¹H NMR: (400 MHz, DMSO-d₆) δ 11.07 (s, 1H), 8.76 (d, J=5.38 Hz, 1H), 8.60 (s, 1H), 8.37-8.45 (m, 4H), 7.38 (t, J=8.80 Hz, 2H), 3.59 (br. s., 4H), 2.86 (br. s., 2H), 2.68 (br. s., 3H), 1.36 (d, J=6.85 Hz, 6H).

### Step-2: Synthesis of 1-(4-(2-(4-fluorophenyl)-5-isopropylpyrimidin-4-ylamino)-1H-pyrrolo [2, 3-b] pyridin-3-yl)-2-(piperazin-1-yl) ethane-1, 2-dione hydrochloride

To the clear solution of 1-(4-(2-(4-fluorophenyl)-5-isopropylpyrimidin-4-ylamino)-1H-pyrrolo [2, 3-b] pyridin-3-yl)-2-(piperazin-1-yl) ethane-1, 2-dione (0.030 g, 0.061 mmol, 1.0 eq) in 10 ml of ethanol was added 5 ml of 1.23 M solution of HCl in ethanol. Solution was stirred at rt for 10 min then solvent was evaporated under reduced pressure at lower temperature. Similar procedure was repeated for 2 more times and then compound was submitted for lyophilisation to get 0.033 g of 1-(4-(2-(4-fluorophenyl)-5-isopropylpyrimidin-4-ylamino)-1H-pyrrolo [2, 3-b] pyridin-3-yl)-2-(piperazin-1-yl) ethane-1, 2-dione hydrochloride. LCMS (M+1): 488.2; ¹H NMR: (400 MHz, DMSO-d₆) δ 11.28 (s, 1H), 9.59 (br. s., 2H), 8.74 (d, J=5.70 Hz, 1H), 8.63 (s, 1H), 8.59 (s, 1H), 8.46 (d, J=6.14 Hz, 1H), 8.40-8.45 (m, 2H), 7.42 (t, J=8.77 Hz, 2H), 3.88 (br. s., 2H), 3.68 (br. s., 2H), 3.58 (br. s., 1H), 3.31 (br. s., 2H), 3.05 (br. s., 2H), 1.38 (d, J=6.58 Hz, 6H).

### Example 24: Preparation of 1-(4-(2-(4-fluorophenyl)-5-isopropylpyrimidin-4-ylamino)-1H-pyrrolo [2, 3-b] pyridin-3-yl)-2-(4-methylpiperazin-1-yl) ethane-1,2-dione hydrochloride (Compound No. 24)

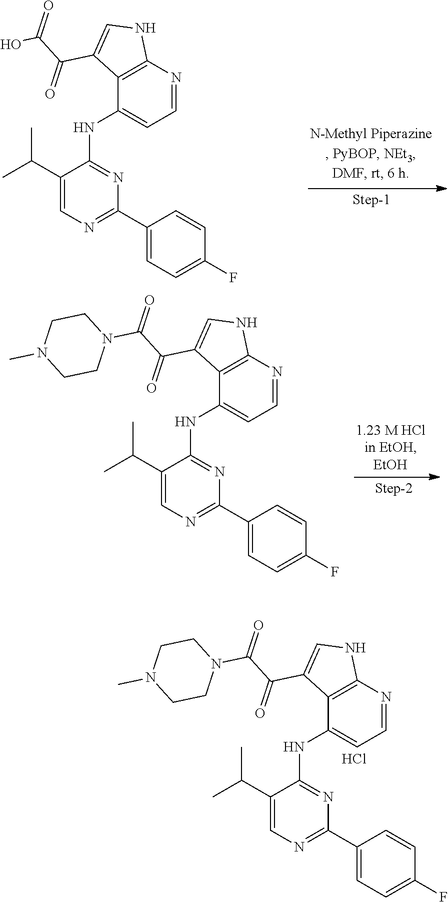

### Step-1: Synthesis of 1-(4-(2-(4-fluorophenyl)-5-isopropylpyrimidin-4-ylamino)-1H-pyrrolo [2,3-b] pyridin-3-yl)-2-(4-methylpiperazin-1-yl) ethane-1,2-dione

To solution of 2-(4-(2-(4-fluorophenyl)-5-isopropylpyrimidin-4-ylamino)-1H-pyrrolo [2, 3-b]pyridin-3-yl)-2-oxoacetic (0.150 g, 0.357 mmol, 1.0 eq) in 15 ml of DMF were added NEt₃ (0.15 ml, 1.07 mmol, 3.0 eq) and PyBOP (0.240 g, 0.464 mmol, 1.3 eq) at it. Reaction mass was allowed to stirred at rt for 15 min and then N-methyl piperazine (0.08 ml, 0.714 mmol, 2.0 eq) was added slowly drop wise. Reaction was stirred for 6 h under N₂ atm. After completion of reaction DMF was removed by high vacuum distillation and product was purified by PREP-HPLC to get 0.035 g of 1-(4-(2-(4-fluorophenyl)-5-isopropylpyrimidin-4-ylamino)-1H-pyrrolo [2, 3-b] pyridin-3-yl)-2-(4-methylpiperazin-1-yl) ethane-1, 2-dione as white solid. Desired product was confirmed by 1H-NMR. LCMS (M+1): 502.3; ¹H NMR (400 MHz, DMSO-d₆) δ 13.22 (br. s., 1H), 11.02 (br. s., 1H), 8.79 (d, J=5.70 Hz, 1H), 8.61 (s, 1H), 8.46-8.38 (m, 4H), 7.39 (t, J=8.77 Hz, 2H), 3.62 (m, 5H), 2.85-2.75 (m, 4H), 2.62 (s, 3H), 1.38 (d, J=7.02 Hz, 6H).

### Step-2: Synthesis of 1-(4-(2-(4-fluorophenyl)-5-isopropylpyrimidin-4-ylamino)-1H-pyrrolo [2, 3-b] pyridin-3-yl)-2-(4-methylpiperazin-1-yl) ethane-1, 2-dione hydrochloride

To the clear solution of 1-(4-(2-(4-fluorophenyl)-5-isopropylpyrimidin-4-ylamino)-1H-pyrrolo [2, 3-b] pyridin-3-yl)-2-(4-methylpiperazin-1-yl) ethane-1, 2-dione (0.035 g, 0.0698 mmol, 1.0 eq) in 10 ml of ethanol was added 5 ml of 1.23 M solution of HCl in ethanol. Reaction mixture was stirred at rt for 10 min then solvent was evaporated under reduced pressure at lower temperature. Similar procedure was repeated twice and then compound was submitted for lyophilization to get 0.035 g of 1-(4-(2-(4-fluorophenyl)-5-isopropylpyrimidin-4-ylamino)-1H-pyrrolo [2, 3-b] pyridin-3-yl)-2-(4-methylpiperazin-1-yl) ethane-1, 2-dione hydrochloride. LCMS (M+1): 502.3; ¹H NMR (400 MHz, DMSO-d₆) δ 11.22 (br. s., 1H), 8.75 (d, J=5.70 Hz, 1H), 8.60 (s, 1H), 8.57 (s, 1H), 8.45 (d, J=6.14 Hz, 1H), 8.39-8.44 (m, 2H), 7.42 (t, J=8.77 Hz, 2H), 4.49 (d, J=13.15 Hz, 2H), 3.92 (d, J=15.35 Hz, 3H), 3.50-3.64 (m, 4H), 3.37 (d, J=14.47 Hz, 2H), 3.10-2.99 (m, 1H), 2.80 (br. s., 3H), 1.38 (d, J=7.02 Hz, 6H).

### Example 25: Preparation of 2-(4-((5-isopropyl-2-(4-methoxyphenyl)

pyrimidin-4-yl) amino)-1H-pyrrolo[2,3-b]pyridin-3-yl)-2-oxoacetamide hydrochloride (Compound No. 25)

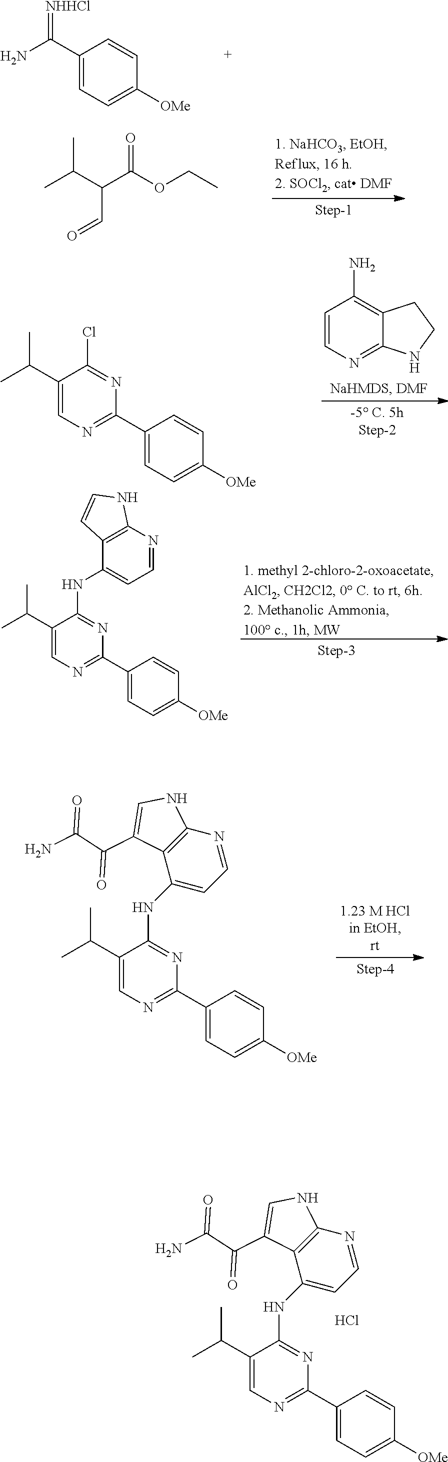

### Step-1: Synthesis of 4-chloro-5-isopropyl-2-(4-methoxyphenyl) pyrimidine

To the solution of 4-methoxybenzimidamide hydrochloride (2.0 g, 10.70 mmol, 1.0 eq.) in ethanol (50 ml), ethyl 2-formyl-3-methylbutanoate (2.2 g, 13.91 mmol, 1.3 eq.) was added followed by NaHCO3 (2.6 g, 32.13 mmol, 3.0 eq). Reaction mixture was refluxed at 85° C. for 16 h. The progress of reaction was monitored by TLC and LCMS. Upon completion of reaction ethanol was removed under reduced pressure to get crude product. Crude material was triturated with n-pentane to give 3.0 g of 5-isopropyl-2-(4-methoxyphenyl) pyrimidin-4-ol. To the suspension of 5-isopropyl-2-(4-methoxyphenyl) pyrimidin-4-ol (3.0 g, 12.23 mmol) in 14 ml thionyl chloride taken in 100 ml sealed tube was added 2-3 drops of DMF. Reaction mass was heated at 90° C. for 2 h. Progress of reaction was monitored by TLC. Upon completion of reaction thionyl chloride was removed under reduced pressure. Remaining was dissolved in ethyl acetate 250 ml, and then organic layer was washed with 10% NaHCO3 twice to remove remaining thionyl chloride followed by water and brine solution. Dried over anhydrous Na2SO4, and solvent was evaporated under reduced pressure at low temperature to obtain 2.0 g of 4-chloro-5-isopropyl-2-(4-methoxyphenyl)pyrimidine as yellow solid. LCMS (M+1): 263.1; ¹H NMR (400 MHz, DMSO-d₆) δ 8.83 (s, 1H), 8.23-8.29 (m, J=8.80 Hz, 2H), 7.04-7.10 (m, J=9.29 Hz, 2H), 3.84 (s, 3H), 3.16-3.24 (m, 1H), 1.30 (d, J=6.85 Hz, 6H).

### Step-2; Synthesis of N-(5-isopropyl-2-(4-methoxyphenyl) pyrimidin-4-yl)-1H-pyrrolo[2,3-b]pyridin-4-amine

To the solution of compound 4-amino 7-azaindole (0.608 g, 4.56 mmol, 1.0 eq) in 5 ml DMF cooled at −5° were added 4-chloro-5-isopropyl-2-(4-methoxyphenyl)pyrimidine (1.2 g, 4.56 mmol, 1.0 eq) followed by slow drop wise addition of solution of NaHMDS (14 ml, 27.39 mmol, 35% in THF) in two lots, under N2 atm. Reaction mass was continued at same temperature for 5 h. After completion of reaction (TLC monitoring), reaction mass was poured in 1 lit of ice cooled water. Resulting solid was filtered out to get 1.5 g of crude product. Product was purified by column chromatography (100-200) mesh silica; product was eluted in 3% MeOH:DCM. Wt=0.7 g, white solid. LCMS (M+1): 360.2; ¹H NMR (400 MHz, DMSO-d₆) δ 11.55 (br. s., 1H), 8.66 (s, 1H), 8.44 (s, 1H), 8.16-8.20 (m, 2H), 8.14 (s, 1H), 7.56 (d, J=5.38 Hz, 1H), 7.32-7.37 (m, 1H), 6.98 (d, J=8.80 Hz, 2H), 6.47 (dd, J=1.71, 3.18 Hz, 1H), 3.79 (s, 3H), 3.42-3.50 (m, 1H), 1.32 (d, J=6.85 Hz, 6H).

### Step-3; Synthesis of 2-(4-((5-isopropyl-2-(4-methoxyphenyl) pyrimidin-4-yl) amino)-1H-pyrrolo[2,3-b]pyridin-3-yl)-2-oxoacetamide

To the solution of N-(5-isopropyl-2-(4-methoxyphenyl) pyrimidin-4-yl)-1H-pyrrolo[2,3-b]pyridin-4-amine (0.7 g, 1.94 mmol, 1.0 eq) in 30 ml CH₂Cl₂ cooled at 0° C. were added aluminum trichloride (1.3 g, 9.70 mmol, 5.0 eq) and methyl 2-chloro-2-oxoacetate (1.7 ml, 19.47 mmol, 10 eq, d=1.33 g/ml) under N₂ atmosphere. Reaction mass was allowed to warm at room temperature and then stirred for 48 h. After completion reaction (LCMS monitoring) methanol (50 ml) was added to reaction mass and again it was stirred at overnight. Then methanol was removed by distillation and crude was taken in 250 ml of ethyl acetate. Organic layer was washed by water twice (2×100 ml) and brine solution twice (2×100 ml). Dried over anhydrous Na₂SO₄, and solvent was evaporated under reduced pressure at low temperature to obtain 0.8 g of methyl 2-(4-((5-isopropyl-2-(4-methoxyphenyl) pyrimidin-4-yl) amino)-1H-pyrrolo [2, 3-b]pyridin-3-yl)-2-oxoacetate as yellow solid. Solution of methyl 2-(4-((5-isopropyl-2-(4-methoxyphenyl) pyrimidin-4-yl) amino)-1H-pyrrolo [2, 3-b] pyridin-3-yl)-2-oxoacetate (0.100 g, 0.224 mmol, 1.0 eq) in 7 ml of methanolic ammonia was heated at 100° C. for 1 h in microwave. After completion of reaction methanol was removed by distillation and crude was purified by PREP-HPLC to give 0.035 g of 2-(4-((5-isopropyl-2-(4-methoxyphenyl) pyrimidin-4-yl) amino)-1H-pyrrolo[2,3-b]pyridin-3-yl)-2-oxoacetamide. LCMS (M+1): 431.2; ¹H NMR: (400 MHz, DMSO-d₆) δ 11.21 (s, 1H), 8.75 (d, J=5.70 Hz, 1H), 8.67 (s, 1H), 8.56 (s, 1H), 8.30-8.38 (m, 3H), 7.96 (br. s., 1H), 7.10 (d, J=9.21 Hz, 2H), 6.54 (s, 2H), 3.84 (s, 3H), 3.55-3.61 (m, 1H), 1.37 (d, J=6.58 Hz, 6H).

### Step-4; Synthesis of 2-(4-((5-isopropyl-2-(4-methoxyphenyl) pyrimidin-4-yl) amino)-1H-pyrrolo[2,3-b]pyridin-3-yl)-2-oxoacetamide hydrochloride

To the clear solution of 2-(4-((5-isopropyl-2-(4-methoxyphenyl) pyrimidin-4-yl) amino)-1H-pyrrolo[2,3-b]pyridin-3-yl)-2-oxoacetamide (0.035 g, 0.081 mmol, 1.0 eq) in 10 ml of ethanol was added 5 ml of 1.23 M solution of HCl in ethanol. Reaction mixture was stirred at rt for 10 min then solvent was evaporated under reduced pressure at lower temperature. Similar procedure was repeated twice and then residue was lyophilized to get 0.035 g of 2-(4-(5-isopropyl-2-(4-methoxyphenyl)pyrimidin-4-yl)amino)-1H-pyrrolo[2,3-b]pyridin-3-yl)-2-oxoacetamide hydrochloride. LCMS (M+1): 431.2; ¹H NMR: (400 MHz, DMSO-d₆) δ 11.69 (br. s., 1H), 8.78 (s, 1H), 8.69 (d, J=5.70 Hz, 1H), 8.46 (s, 1H), 8.45 (br. s., 1H), 8.31-8.37 (m, J=8.77 Hz, 2H), 8.26 (br. s., 1H), 8.00 (br. s., 1H), 7.14-7.20 (m, J=8.77 Hz, 2H), 3.88 (s, 3H), 3.59-3.63 (m, 1H), 1.39 (d, J=7.02 Hz, 6H).

### Example 26; Preparation of 2-(4-((5-isopropyl-2-phenylpyrimidin-4-yl)amino)-1H-pyrrolo[2,3-b]pyridin-3-yl)-2-oxoacetamide hydrochloride (Compound No. 26)

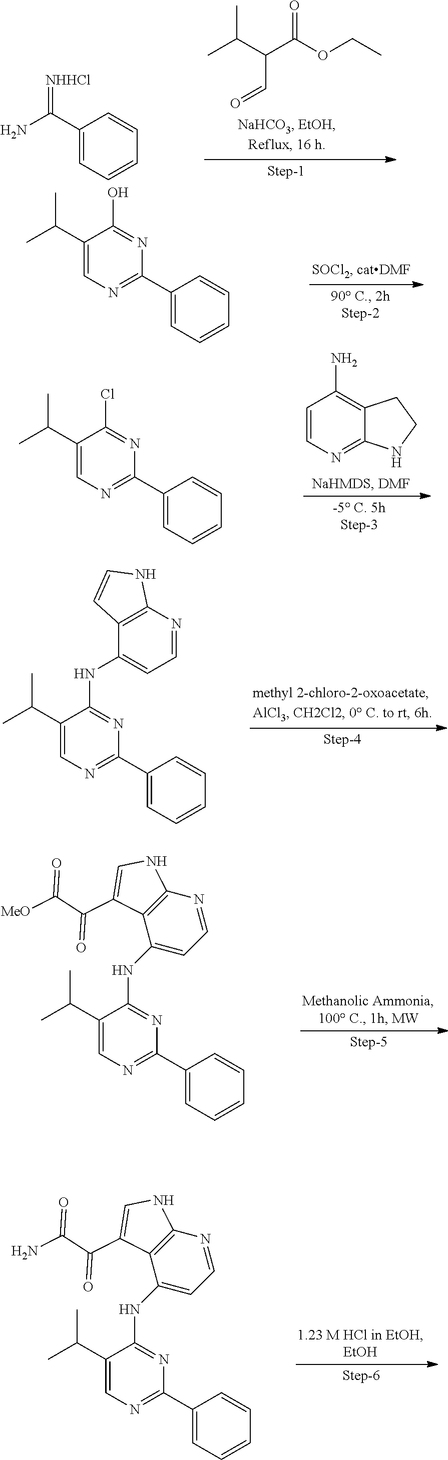

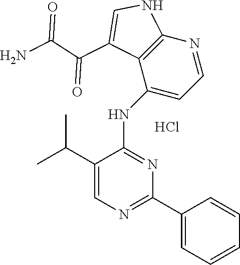 [SMILES: CC(C)c1cnc(-c2ccccc2)nc1Nc1ccnc2[nH]cc(C(=O)C(N)=O)c12 ; Cl]

### Step-1: Synthesis of 5-isopropyl-2-(4-methoxyphenyl) pyrimidin-4-ol

To the solution of benzimidamide hydrochloride (2.0 g, 12.8 mmol, 1.0 eq.) and ethyl 2-formyl-3-methylbutanoate (2.02 g, 12.8 mmol, 1 eq.) in ethanol (40 ml) was added NaHCO₃ (3.23 g, 38.4 mmol, 3.0 eq.). Reaction mixture was refluxed at 85° C. for 16 h. The progress of reaction was monitored by TLC and LCMS. Upon completion the ethanol was removed under reduced pressure to get crude product. Crude material was triturated with n-pentane to give 2.5 g of 5-isopropyl-2-phenylpyrimidin-4-ol. LCMS (M+1): 215.11

### Step-2: Synthesis of 4-chloro-5-isopropyl-2-phenylpyrimidine

To the suspension of 5-isopropyl-2-(4-methoxyphenyl) pyrimidin-4-ol (1.2 g, 5.6 mmol) in 12 ml thionyl chloride taken in 100 ml sealed tube was added 2-3 drops of DMF. Reaction mixture was heated at 90° C. for 2 h. Progress of reaction was monitored by TLC. Upon completion of reaction thionyl chloride was removed under reduced pressure. Remaining was dissolved in ethyl acetate 250 ml, and then organic layer was washed with 10% NaHCO3 twice to remove remaining thionyl chloride followed by water and brine solution. Dried over anhydrous Na2SO4, and solvent was evaporated under reduced pressure at low temperature to obtain 1.1 g of 4-chloro-5-isopropyl-2-phenylpyrimidine as white solid. LCMS (M+1): 233.1; ¹H NMR: (400 MHz, CHLOROFORM-d) δ 8.64 (s, 1H), 8.41 (dd, J=2.69, 6.60 Hz, 2H), 7.40-7.53 (m, 3H), 3.27-3.38 (m, 1H), 1.32-1.40 (m, 6H).

### Step-3; Synthesis of N-(5-isopropyl-2-phenylpyrimidin-4-yl)-1H-pyrrolo [2,3-b]pyridin-4-amine

To the solution of compound 4-amino 7-azaindole (0.550 g, 4.1 mmol, 1.0 eq.) in 2 ml DMF cooled at −5° C. and then 4-chloro-5-isopropyl-2-phenylpyrimidine (0.959 g, 4.1 mmol, 1.0 eq.) was added followed by drop wise addition of solution of NaHMDS (14 ml, 27.39 mmol, 35% in THF) in two lots, under N₂ atm. Reaction mixture was continued at same temperature for 5 h. After completion of reaction (TLC monitoring), reaction mass was poured in 1 lit of ice cooled water. Resulting solid was filtered out to get 1.5 g of crude compound of N-(5-isopropyl-2-phenylpyrimidin-4-yl)-1H-pyrrolo [2,3-b]pyridin-4-amine as white solid. LCMS (M+1): 330.16

### Step-4; Synthesis of methyl 2-(4-((5-isopropyl-2-phenylpyrimidin-4-yl)amino)-1H-pyrrolo[2,3-b]pyridin-3-yl)-2-oxoacetate

To the solution of N-(5-isopropyl-2-phenylpyrimidin-4-yl)-1H-pyrrolo[2,3-b]pyridin-4-amine (0.43 g, 1.3 mmol, 1.0 eq.) in 25 ml CH₂Cl₂ cooled at 0° C. was added aluminum trichloride (0.87 g, 6.5 mmol, 5.0 eq.) and methyl 2-chloro-2-oxoacetate (2.35 g, 21.6 mmol, 16.6 eq.) under N₂ atmosphere. Reaction mixture was allowed to warm at room temperature and then stirred for 48 h. After completion reaction (LCMS monitoring) methanol (50 ml) was added to reaction mixture at 0° C. and again it was stirred at room temperature for 18 h. Organic solvent was removed by distillation and crude was taken in 250 ml of ethyl acetate. Organic layer was washed by water twice (2×100 ml) and brine solution twice (2×100 ml). Dried over anhydrous Na2SO4, and solvent was evaporated under reduced pressure at low temperature to obtain 0.25 g of methyl 2-(4-((5-isopropyl-2-phenylpyrimidin-4-yl)amino)-1H-pyrrolo[2,3-b]pyridin-3-yl)-2-oxoacetate as light yellow solid compound. LCMS (M+1): 416.16

### Step-5; Synthesis of 2-(4-((5-isopropyl-2-phenylpyrimidin-4-yl)amino)-1H-pyrrolo[2,3-b]pyridin-3-yl)-2-oxoacetamide

Solution of crude methyl 2-(4-((5-isopropyl-2-(4-methoxyphenyl) pyrimidin-4-yl) amino)-1H-pyrrolo [2, 3-b] pyridin-3-yl)-2-oxoacetate (0.2 g, 0.4 mmol, 1.0 eq.) in 3 ml of methanolic ammonia was heated at 100° C. for 1 h in microwave. Solvent was evaporated and above procedure was repeated once. After completion of reaction methanol was removed by distillation and crude was purified by PREP-HPLC to give 9 mg of 2-(4-((5-isopropyl-2-phenylpyrimidin-4-yl)amino)-1H-pyrrolo[2,3-b]pyridin-3-yl)-2-oxoacetamide. LCMS (M+1): 401.2; ¹H NMR: (400 MHz, DMSO-d6) δ 11.28 (s, 1H), 8.76 (d, J=5.87 Hz, 1H), 8.68 (s, 1H), 8.61 (s, 1H), 8.34-8.42 (m, 3H), 8.22 (br. s., 1H), 7.94 (br. s., 1H), 7.51-7.59 (m, 3H), 3.59-3.64 (m, 1H), 1.38 (d, J=6.65 Hz, 6H).

### Step-6: Synthesis of 2-(4-((5-isopropyl-2-phenylpyrimidin-4-yl)amino)-1H-pyrrolo[2,3-b]pyridin-3-yl)-2-oxoacetamide hydrochloride

To the clear solution of 2-(4-((5-isopropyl-2-phenylpyrimidin-4-yl)amino)-1H-pyrrolo[2,3-b]pyridin-3-yl)-2-oxoacetamide (9 mg, 0.025 mmol, 1.0 eq.) in 2 ml of ethanol was added 2 ml of 1.23 M solution of HCl in ethanol. Reaction mixture was stirred at rt for 10 min and solvent was evaporated under reduced pressure at lower temperature. Similar procedure was repeated twice, residue was submitted for lyophilization to get 7.5 mg of 2-(4-((5-isopropyl-2-phenylpyrimidin-4-yl)amino)-1H-pyrrolo[2,3-b]pyridin-3-yl)-2-oxoacetamide hydrochloride. LCMS (M+1): 401.2; ¹H NMR: (400 MHz, DMSO-d6) δ 11.40 (s, 1H), 8.76 (d, J=6.14 Hz, 1H), 8.72 (s, 1H), 8.61 (s, 1H), 8.34-8.43 (m, 3H), 8.25 (br. s., 1H), 7.98 (br. s., 1H), 7.54-7.60 (m, 3H), 3.59-3.63 (m, 1H), 1.39 (d, J=7.02 Hz, 6H).

### Example 27: Preparation of 2-(4-(6-(4-fluorophenyl)-3-isopropylpyridin-2-ylamino)-1H-pyrrolo [2,3-b] pyridin-3-yl)-2-oxoacetamide hydrochloride (Compound No. 27)

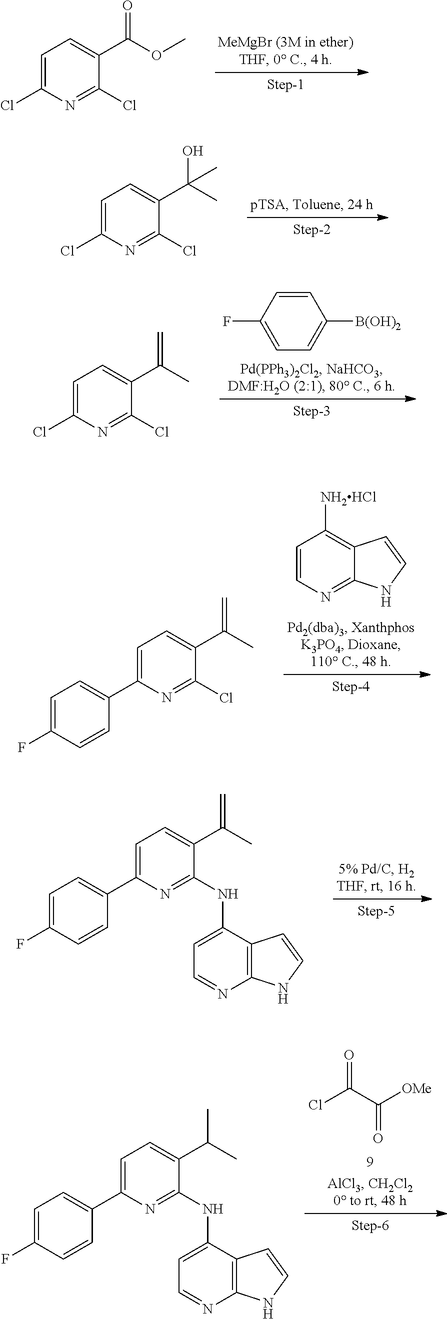

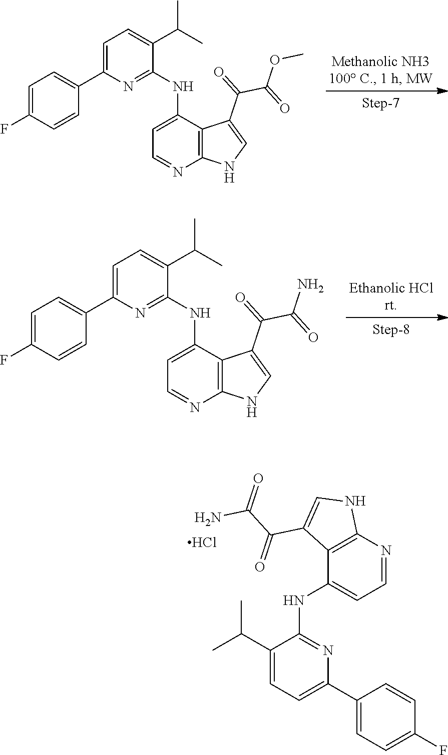

### Step-1: Synthesis of 2-(2, 6-dichloropyridin-3-yl) propan-2-ol

To solution of methyl 2, 6-dichloronicotinate (4.5 g, 22 mmol, 1.0 eq) in 40 ml of THF cooled at 0° C. was added solution of MeMgBr (25.4 ml, 76 mmol, 3.5 eq, 3 M in ether) under nitrogen atmosphere. Reaction mass was stirred at same temperature for 4 h. After completion of reaction (TLC monitoring) excess MeMgBr was quenched by saturated NH₄Cl solution. Desired product was extracted from aq. in ethyl acetate twice (2×100 ml). Organic layer was washed with brine (2×50 ml) and water (2×50 ml). Dried over anhydrous Na₂SO₄, and solvent was evaporated to obtain 4.5 g 2-(2, 6-dichloropyridin-3-yl) propan-2-ol as pale yellow compound. LCMS (M+1): 206.0.

### Step-2: Synthesis of 2, 6-dichloro-3-(prop-1-en-2-yl) pyridine

To solution of methyl 2-(2, 6-dichloropyridin-3-yl) propan-2-ol (4.5 g, 21 mmol, 1.0 eq) in 100 ml of Toluene was added pTSA (4.1 g, 24 mmol, 1.1 eq) under nitrogen atmosphere. Reaction mass was heated to reflux under dean stark condition to remove water. After completion of reaction toluene was removed by distillation and then crude product was dissolved in ethyl acetate 250 ml. Organic layer was washed with brine (2×50 ml) and water (2×50 ml). Dried over anhydrous Na₂SO₄, and solvent was evaporated and crude was purified by column chromatography (100-200) mesh silica, eluent 5-10% EtOAc:Hexane to obtain 3.6 g 2, 6-dichloro-3-(prop-1-en-2-yl) pyridine as pale yellow liquid. ¹H NMR: (400 MHz, CHLOROFORM-d) δ 7.50 (d, J=7.89 Hz, 1H), 7.24-7.28 (m, 1H), 5.32 (s, 1H), 5.05 (s, 1H), 2.10 (s, 3H).

### Step-3: Synthesis of 2-chloro-6-(4-fluorophenyl)-3-(prop-1-en-2-yl) pyridine

Solution of (4-fluorophenyl)boronic acid (1.1 g, 8.01 mmol, 1.0 eq), 2,6-dichloro-3-(prop-1-en-2-yl) pyridine (1.5 g, 8.01 mmol, 1.0 eq) and NaHCO₃ (2.0 g, 24.03 mmol, 3.0 eq) in 60 ml of DMF and 15 ml of water was purged with N₂ gas for 15 min. After completion of purging Pd(PPh₃)₂Cl₂ (0.281 g, 0.40 mmol, 0.05 eq) was added and reaction mass was again purged with N₂ gas for 15 min. Then reaction mass was heated at 80° for 6 h. After completion of reaction, reaction mass was poured in 600 ml of ice water and product was extracted in ethyl acetate. Washed with brine solution twice (2×100 ml), dried over anhydrous Na₂SO₄ and solvent was evaporated and crude was purified by column chromatography (100-200) mesh silica, eluent 5-10% EtOAc: Hexane to obtain 1.6 g of 2-chloro-6-(4-fluorophenyl)-3-(prop-1-en-2-yl) pyridine as pale yellow solid. LCMS (M+1): 248.1

### Step-4: Synthesis of N-(6-(4-fluorophenyl)-3-(prop-1-en-2-yl) pyridin-2-yl)-1H-pyrrolo[2,3-b]pyridin-4-Amine

Solution of 2-chloro-6-(4-fluorophenyl)-3-(prop-1-en-2-yl) pyridine (1.2 g, 4.84 mmol, 1.0 eq), 1H-pyrrolo[2,3-b]pyridin-4-amine (0.646 g, 4.84 mmol, 1.0 eq) and K3PO4 (2.0 g, 9.46 mmol, 2.0 eq) in 20 ml of dioxane was purged with N2 gas for 15 min. After completion of purging Pd2 (dba)3 (0.665 g, 0.726 mmol, 0.15 eq) and Xanthphos (0.420 g, 0.726 mmol, 0.15 mmol) was added and reaction mass was again purged with N2 gas for 15 min. Then reaction mass was heated at 110° C. for 48 h. After completion of reaction, reaction mass was poured in 200 ml of ice water and product was extracted in ethyl acetate. Washed with brine solution twice (2×100 ml), dried over anhydrous Na2SO4 and solvent was evaporated and crude was purified by column chromatography (100-200) mesh silica, eluent 40-50% EtOAc:Hexane to obtain 1.0 g of N-(6-(4-fluorophenyl)-3-(prop-1-en-2-yl) pyridin-2-yl)-1H-pyrrolo[2,3-b]pyridin-4-Amine as pale white solid. LCMS (M+1): 344.9

### Step-5: Synthesis of N-(6-(4-fluorophenyl)-3-isopropylpyridin-2-yl)-1H-pyrrolo[2,3-b]pyridin-4-amine

To solution of N-(6-(4-fluorophenyl)-3-isopropylpyridin-2-yl)-1H-pyrrolo[2,3-b]pyridin-4-amine (1.0 g, 2.90 mmol, 1.0 eq) in 100 ml of THF was added Pd/C (300 Mg, 10%). Reaction mass was stirred at rt under H₂ atmosphere for 16 h. After completion of reaction (1H-NMR monitoring), reaction mass was filtered through celite bed and filtrate was concentrated to get 0.9 g of N-(6-(4-fluorophenyl)-3-isopropylpyridin-2-yl)-1H-pyrrolo[2,3-b]pyridin-4-amine as dark solid. ¹H NMR: (400 MHz, DMSO-d₆) δ 11.38 (br. s., 1H), 8.32 (s, 1H), 7.96-8.09 (m, 3H), 7.74-7.79 (m, 1H), 7.62 (d, J=7.89 Hz, 1H), 7.25-7.31 (m, 2H), 7.20-7.25 (m, 2H), 6.46-6.50 (m, 1H), 3.38-3.46 (m, 1H), 1.19-1.27 (m, 6H).

### Step-6: Synthesis of methyl 2-(4-(6-(4-fluorophenyl)-3-isopropylpyridin-2-ylamino)-1H-pyrrolo [2, 3-b] pyridin-3-yl)-2-oxoacetate

To the solution of methyl 2-(4-(6-(4-fluorophenyl)-3-isopropylpyridin-2-ylamino)-1H-pyrrolo[2,3-b]pyridin-3-yl)-2-oxoacetate (0.2 g, 0.57 mmol, 1.0 eq) in 30 ml CH2Cl2 cooled at 0° C. were added aluminum trichloride (0.384 g, 2.88 mmol, 5.0 eq) and methyl 2-chloro-2-oxoacetate (0.50 ml, 5.77 mmol, 10 eq, d=1.33 g/ml) under N2 atmosphere. Reaction mass was allowed to warm at room temperature and then stirred for 48 h. After completion reaction (LCMS monitoring) methanol (50 ml) was added to reaction mass and again it was stirred for overnight. Then methanol was removed by distillation and crude was taken in 250 ml of ethyl acetate. Organic layer was washed by water twice (2×100 ml) and brine solution twice (2×100 ml). Dried over anhydrous Na₂SO₄, and solvent was evaporated under reduced pressure. Crude product was purified by flash column chromatography to give 0.3 g of methyl 2-(4-(6-(4-fluorophenyl)-3-isopropylpyridin-2-ylamino)-1H-pyrrolo [2,3-b] pyridin-3-yl)-2-oxoacetate.

### Step-7: Synthesis of 2-(4-(6-(4-fluorophenyl)-3-isopropylpyridin-2-ylamino)-1H-pyrrolo [2,3-b] pyridin-3-yl)-2-oxoacetamide

Solution of methyl 2-(4-(6-(4-fluorophenyl)-3-isopropylpyridin-2-ylamino)-1H-pyrrolo [2, 3-b] pyridin-3-yl)-2-oxoacetate (0.100 g, 0.231 mmol, 1.0 eq) in 15 ml of methanolic ammonia was heated at 100° C. for 1 h in microwave. After completion of reaction methanol was removed by distillation and crude was purified by PREP-HPLC to give 0.025 g of 2-(4-(6-(4-fluorophenyl)-3-isopropylpyridin-2-ylamino)-1H-pyrrolo [2,3-b] pyridin-3-yl)-2-oxoacetamide. LCMS (M+1): 418.2

### Step-8: Synthesis of 2-(4-(6-(4-fluorophenyl)-3-isopropylpyridin-2-ylamino)-1H-pyrrolo [2, 3-b] pyridin-3-yl)-2-oxoacetamide hydrochloride

To the clear solution of 2-(4-(6-(4-fluorophenyl)-3-isopropylpyridin-2-ylamino)-1H-pyrrolo [2, 3-b]pyridin-3-yl)-2-oxoacetamide (0.025 g, 0.059 mmol, 1.0 eq) in 10 ml of ethanol was added 5 ml of 1.23M solution of HCl in ethanol. Solution was stirred at rt for 10 min then solvent was evaporated under reduced pressure at lower temperature. Similar procedure was repeated for 2 more times and then compound was submitted for lypholisation to get 0.025 g of 2-(4-(6-(4-fluorophenyl)-3-isopropylpyridin-2-ylamino)-1H-pyrrolo [2, 3-b] pyridin-3-yl)-2-oxoacetamide hydrochloride. LCMS (M+1): 418.2; ¹H NMR: (400 MHz, DMSO-d₆) δ 11.30 (br. s., 1H), 8.68 (s, 1H), 8.28 (br. s., 1H), 8.26 (s, 2H), 8.13 (dd, J=5.48, 9.00 Hz, 2H), 8.01 (br. s., 1H), 7.90 (d, J=7.83 Hz, 1H), 7.75 (d, J=7.83 Hz, 1H), 7.34 (t, J=9.00 Hz, 2H), 3.47-3.52 (m, 2H), 1.33 (d, J=6.65 Hz, 6H).

### Example 28: Preparation of 2-(4-(2-(4-fluorophenyl)-5-isopropylpyrimidin-4-ylamino)-1H-indol-3-yl)-2-oxoacetamide hydrochloride (Compound No. 28)

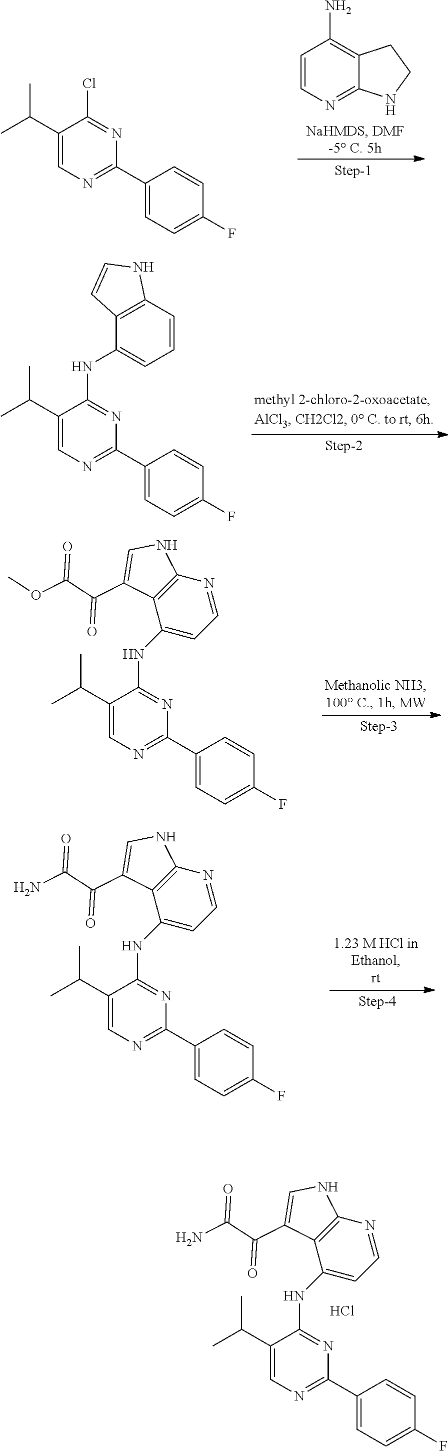

### Step-1: Synthesis of N-(2-(4-fluorophenyl)-5-isopropylpyrimidin-4-yl)-1H-indol-4-amine

To the solution of compound 1H-indol-4-amine (1.04 g, 7.91 mmol, 1.1 eq) in 5 ml DMF cooled at −5° C. were added 4-chloro-2-(4-fluorophenyl)-5-isopropylpyrimidine (1.8 g, 7.19 mmol) followed by slow drop wise addition of NaHMDS solution (22.7 ml, 43.14 mmol, 35% in THF) in two lots, under N₂ atmosphere. Reaction mass was continued at same temperature for 5 h. After completion of reaction (TLC monitoring), reaction mass was poured in 500 ml of ice cooled water. Resulting solid product was filtered out, which was further purified by flash column chromatography to give 0.6 g of N-(2-(4-fluorophenyl)-5-isopropylpyrimidin-4-yl)-1H-indol-4-amine as pale pink solid. LCMS (M+1): 347.3;

### Step-2: Synthesis of methyl 2-(4-(2-(4-fluorophenyl)-5-isopropylpyrimidin-4-ylamino)-1H-indol-3-yl)-2-oxoacetate

To the solution of N-(2-(4-fluorophenyl)-5-isopropylpyrimidin-4-yl)-1H-indol-4-amine (0.3 g, 0.866 mmol, 1.0 eq) in 30 ml CH₂Cl₂ cooled at 0° C. were added aluminum trichloride (0.577 g, 4.33 mmol, 5.0 eq) and methyl 2-chloro-2-oxoacetate (0.8 ml, 8.66 mmol, 10 eq, d=1.33 g/ml) under N2 atmosphere. Reaction mass was allowed to warm at room temperature and then stirred for 48 h. After completion reaction (LCMS monitoring) methanol (20 ml) was added to reaction mass and again it was stirred for overnight. Then methanol was removed by distillation and crude material was taken in 100 ml of ethyl acetate. Organic layer were washed by water twice (2×100 ml) and brine solution twice (2×100 ml). Dried over anhydrous Na₂SO₄, and solvent was evaporated under reduced pressure at low temperature to obtain 0.6 g of methyl 2-(4-(2-(4-fluorophenyl)-5-isopropylpyrimidin-4-ylamino)-1H-indol-3-yl)-2-oxoacetate as dark solid. LCMS (M+1): 433.16

### Step-3: Synthesis of 2-(4-(2-(4-fluorophenyl)-5-isopropylpyrimidin-4-ylamino)-1H-indol-3-yl)-2-oxoacetamide

Solution of methyl 2-(4-(2-(4-fluorophenyl)-5-isopropylpyrimidin-4-ylamino)-1H-indol-3-yl)-2-oxoacetate (0.6 g, 1.38 mmol, 1.0 eq) in 10 ml of methanolic ammonia was heated at 100° C. for 1 h in microwave. After completion of reaction methanol was removed by distillation and crude material was purified by PREP-HPLC to give 35 mg of 2-(4-(2-(4-fluorophenyl)-5-isopropylpyrimidin-4-ylamino)-1H-indol-3-yl)-2-oxoacetamide as white solid. ¹H NMR: (400 MHz, DMSO-d₆) δ 12.64 (br. s., 1H), 11.12 (br. s., 1H), 8.62 (d, J=3.07 Hz, 1H), 8.54 (d, J=7.67 Hz, 1H), 8.42 (s, 1H), 8.31-8.38 (m, 2H), 8.19 (br. s., 1H), 7.89 (br. s., 1H), 7.36-7.41 (m, 2H), 7.35 (br. s., 1H), 7.29 (d, J=7.67 Hz, 1H), 3.57-3.65 (m, 2H), 1.37 (d, J=6.58 Hz, 6H).

### Step-4: Synthesis of 2-(4-(2-(4-fluorophenyl)-5-isopropylpyrimidin-4-ylamino)-1H-indol-3-yl)-2-oxoacetamide hydrochloride

2-(4-(2-(4-fluorophenyl)-5-isopropylpyrimidin-4-ylamino)-1H-indol-3-yl)-2-oxoacetamide (0.035 g, 0.083 mmol, 1.0 eq) was completely solubilized in 15 ml of ethanol and HCl in ethanol (5 ml of 1.23 M solution) was added to it. Solution was stirred at rt for 10 min then solvent was evaporated under reduced pressure at lower temperature. Similar procedure was repeated for 2 more times and then compound was submitted for lyophilisation to get 35 mg of 2-(4-(2-(4-fluorophenyl)-5-isopropylpyrimidin-4-ylamino)-1H-indol-3-yl)-2-oxoacetamide hydrochloride. LCMS (M+1): 418.1; ¹H NMR: (400 MHz, DMSO-d6) δ 12.8 (s, 1H), 11.40 (s, 1H), 8.75 (s, 1H), 8.50 (d, 1H), 8.35 (s, 3H), 8.20 (s, 1H), 7.95 (s, 1H), 7.45 (dd, 2H), 7.40 (s, 2H), 3.58-3.63 (m, 1H), 1.30

### Example 29: Preparation of 2-(4-((2-(4-fluorophenyl)-5-isopropylpyrimidin-4-yl)amino)-1H-pyrrolo[2,3-b]pyridin-3-yl)-N-methyl-2-oxoacetamide hydrochloride (Compound No. 29)

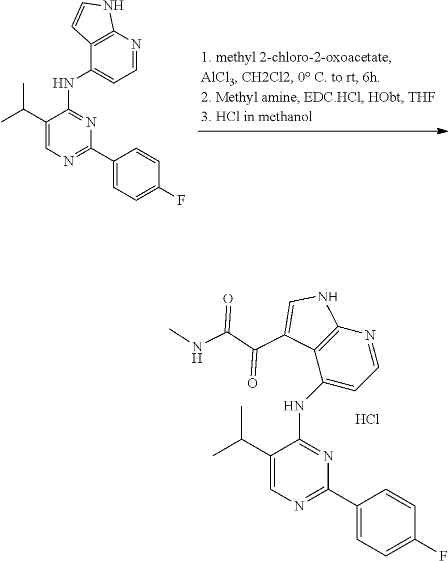 [SMILES: CC(C)c1cnc(-c2ccc(F)cc2)nc1Nc1ccnc2[nH]ccc12 ; CNC(=O)C(=O)c1c[nH]c2nccc(Nc3nc(-c4ccc(F)cc4)ncc3C(C)C)c12 ; Cl]

To the solution of N-(2-(4-fluorophenyl)-5-isopropylpyrimidin-4-yl)-1H-pyrrolo[2,3-b]pyridin-4-amine (0.25 g, 0.72 mmol, 1.0 eq.) in 30 ml CH₂Cl₂ cooled at 0° C. were added aluminum trichloride (0.479 g, 3.6 mmol, 5.0 eq.) and methyl 2-chloro-2-oxoacetate (1.29 g, 11.9 mmol, 16.6 eq. (d=1.33 g/ml)) under N₂ atmosphere. Reaction mass was allowed to warm at room temperature and then stirred for 48 h. After completion reaction (LCMS monitoring) methanol (50 ml) was added to reaction mass and again it was stirred at overnight. Then methanol was removed by distillation and crude was taken in 100 ml of ethyl acetate. Organic layer was washed by water twice (2×100 ml) and brine solution twice (2×100 ml). Dried over anhydrous Na₂SO₄, and solvent was evaporated under reduced pressure at low temperature to obtain 0.4 g of crude material. Crude material was dissolved in THF and its pH was adjusted to 9-10 using 2M aq. NaOH and reaction was stirred for 18 h to get corresponding acid (confirmed by LCMS). Reaction mixture was acidified to pH 1 using aq. HCl and then was extracted with ethyl acetate thrice; the organic layer was evaporated to get crude acid (confirmed by LCMS). The acid (crude 0.3 g, 0.71 mmol, 1 eq.) was dissolved in DMF (5 ml) add to it EDC.HCl (0.275 g, 1.43 mmol, 2 eq.), HOBt (0.009 g, 0.071 mmol, 0.1 eq.) and 2M methyl amine solution (0.221 g, 7.1 mmol, 10 eq.) and reaction was stirred at 50° C. for 24 h in a closed vessel followed by LCMS. Solvent was evaporated and residue was diluted with water and then extracted with ethylacetae three times and separated organic layer was evaporated to get crude product. The crude product was purified by flash chromatography. The product was then treated with 4 M dioxane in HCl and solvent was evaporated. The residue was lyophilized to get 2-(4-((2-(4-fluorophenyl)-5-isopropylpyrimidin-4-yl) amino)-1H-pyrrolo [2,3-b] pyridin-3-yl)-N-methyl-2-oxoacetamide hydrochloride. Product was confirmed by 1H-NMR. ¹H-NMR (400 MHz, DMSO-d6) □ 11.35 (s, 1H), 8.82 (s, 1H), 8.78 (d, J=4.82 Hz, 1H), 8.70 (d, J=5.70 Hz, 1H), 8.61 (s, 1H), 8.38-8.44 (m, 3H), 7.39 (t, J=8.77 Hz, 2H), 3.56-3.63 (m, 1H), 2.81 (d, J=4.82 Hz, 3H), 1.39 (d, J=6.58 Hz, 6H); ¹H NMR D₂O: (400 MHz, DMSO-d6) δ 8.78 (s, 1H), 8.70 (d, J=5.70 Hz, 1H), 8.54 (s, 1H), 8.32-8.41 (m, 3H), 7.36 (t, J=8.77 Hz, 2H), 3.51-3.55 (m, 1H), 1.30-1.38 (m, 6H); LCMS: 433.2[M+H]⁺

The effect of the compounds represented by general Formula III on Transforming Growth Factor Beta Receptor 1 (ALK5) activity and Mitogen-Activated Protein Kinase 14 (P38α) activity in in vitro conditions were investigated.

In Vitro ALK-5 and P38α Inhibitory Activity

ALK5 Kinase Assay for IC₅₀ Determination:

IC₅₀ values of compounds against TGFβ kinase was determined by LanthaScreen™ Terbium Labeled TR-FRET assay. Kinase assays were performed in IX kinase buffer (# PV6135, Invitrogen, Life Technologies Grand Island, N.Y.) where total reaction volume was 10 μL in low-volume 384-well plates (#4511,Corning). Serially diluted compounds (3-fold) were incubated with TGFβ kinase (6 nM) for 10 min, following which a mixture of ATP (10 μM) (# A1852, Sigma, St-Louis, Mo.) and fluorescent-PolyGT substrate (200 nM) (# PV3610, Invitrogen, Life Technologies Grand Island, N.Y.) was added and incubated in dark at room temperature for 1 h. After 1 h, 10 μL stop solution containing Terbium labeled antibody (4 nM) (# PV3529, Invitrogen, Life Technologies Grand Island, N.Y.) and EDTA (# E5134, Sigma, St-Louis, Mo.) (20 mM) in TR-FRET dilution buffer (# PV3574, Invitrogen, Life Technologies Grand Island, N.Y.) was added. Readings were taken in a Synergy Neo Plate reader (BioTek, Winooski) at single excitation of 340 nm and Dual emission at 495 nm and 520 nm respectively.

The % activity of test samples was calculated as (Sample−Min)×100/(Max−Min). [Max: DMSO control, complete reaction with enzyme with DMSO and Min: No enzyme with DMSO]. Percent inhibition (100-% activity) was fitted to the “four-parameter logistic model” in XLfit for determination of IC₅₀ values.

MAPK14/p38α Kinase Assay for IC₅₀ Determination:

IC₅₀ values of compounds against MAPK14/P38α kinase were determined by Lanthascreen based TR-FRET assay. Kinase reactions were performed in a 10 μL volume in low-volume 384-well plates. The concentration of substrate was maintained at 400 nM in the assay, and the kinase reaction buffer (1×; Cat # PV3189, ThermoFisher) consisted of 50 mM HEPES pH 7.5, 0.01% BRIJ-35, 10 mM MgCl2, and 1 mM EGTA. Serially diluted compounds (3-fold) in DMSO (0.5% in final reaction) were incubated with cocktail of MAPK14/p38α kinase (20 ng/ml; Cat # PV3304, ThermoFisher), Substrate GFP-ATF2 (19-96), (0.4 μM; Cat # PV4445, ThermoFisher), ATP (90 μM; Cat # PV3227, ThermoFisher) and buffer. Kinase reactions were allowed to proceed for 1 hour at room temperature before EDTA (10 mM; Cat # P2825, ThermoFisher) and LanthaScreen™ Tb-antipATF2 (pThr71) Antibody (2 nM; Cat # PV4451, ThermoFisher) in TR-FRET dilution buffer (IX; Cat # PV3574, ThermoFisher) were added. The plate was allowed to incubate at room temperature for 30 minutes before being read on a BMG Pherastar plate reader using the LanthaScreen™ filter block for LanthaScreen™ TR-FRET.

The resulting TR-FRET emission ratio was plotted against the concentration of inhibitor, and the data was fit to a sigmoidal dose-response curve with a variable slope. The IC₅₀ for the inhibitor was calculated from the curve.

Table 2 below illustrates percentage Inhibition of enzyme activity (ALK-5 and P38 Alpha MAP kinase) at 100 nM concentration of compounds

| | | Enzyme Activity | Enzyme Activity |
|---|---|---|---|
| | | TGFBR1 | MAPK14 |
| Compound | | (ALK5) | (p38 alpha) |
| No. | Structure of NCE | % Inhibition at 100 nM | % Inhibition at 100 nM |
| 1 | 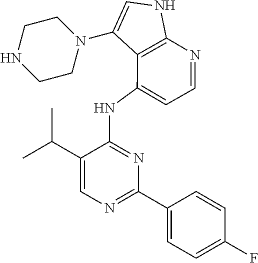 [SMILES: CC(C)c1cnc(-c2ccc(F)cc2)nc1Nc1ccnc2[nH]cc(N3CCNCC3)c12] | 99 | 61 |
| 2 | 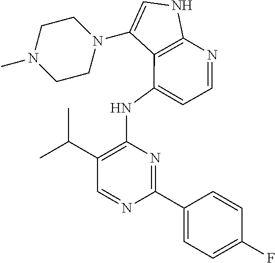 [SMILES: CC(C)c1cnc(-c2ccc(F)cc2)nc1Nc1ccnc2[nH]cc(N3CCN(C)CC3)c12] | 84 | 19 |
| 3 | 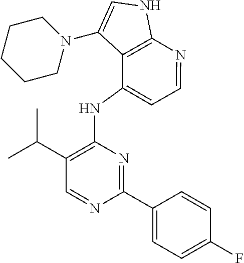 [SMILES: CC(C)c1cnc(-c2ccc(F)cc2)nc1Nc1ccnc2[nH]cc(N3CCCCC3)c12] | 72 | 42 |
| 4 | 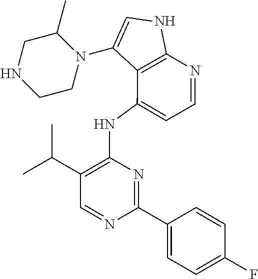 [SMILES: CC(C)c1cnc(-c2ccc(F)cc2)nc1Nc1ccnc2[nH]cc(N3CCNCC3C)c12] | 94 | 72 |
| 5 | 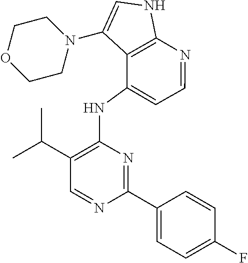 [SMILES: CC(C)c1cnc(-c2ccc(F)cc2)nc1Nc1ccnc2[nH]cc(N3CCOCC3)c12] | 88 | 7 |
| 6 | 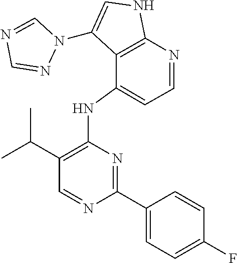 [SMILES: CC(C)c1cnc(-c2ccc(F)cc2)nc1Nc1ccnc2[nH]cc(-n3cncn3)c12] | 97 | 0 |

Table 3 below illustrates IC₅₀ value of dual ALK-5 and P38 Alpha MAP kinase inhibitor compounds

| Compound | | IC₅₀ (ALK5) | IC₅₀ (P38 alpha) |
|---|---|---|---|
| No. | Structure of NCE | (μM) | (μM) |
| 7 | 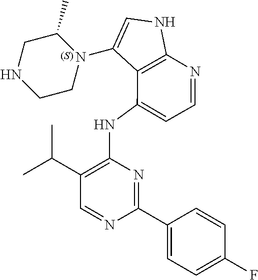 | 0.155 | 0.042 |
| 8 | 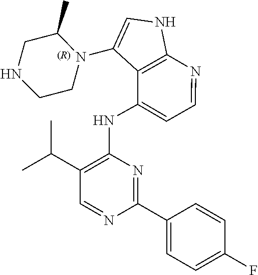 | 0.095 | 0.061 |
| 17 | 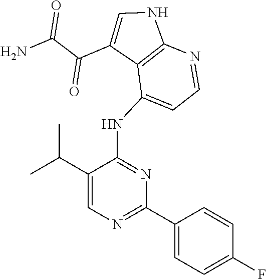 [SMILES: CC(C)c1cnc(-c2ccc(F)cc2)nc1Nc1ccnc2[nH]cc(C(=O)C(N)=O)c12] | >5 | 0.309 |
| 18 | 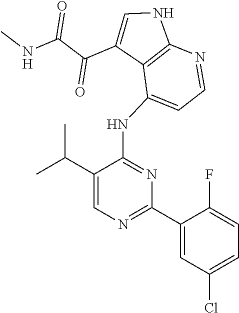 [SMILES: CNC(=O)C(=O)c1c[nH]c2nccc(Nc3nc(-c4cc(Cl)ccc4F)ncc3C(C)C)c12] | >5 | 0.349 |
| 19 | 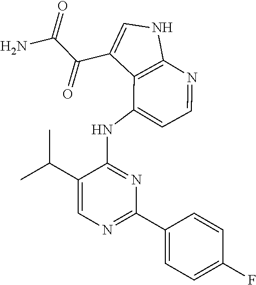 [SMILES: CC(C)c1cnc(-c2ccc(F)cc2)nc1Nc1ccnc2[nH]cc(C(=O)C(N)=O)c12] | 0.278 | 1.76 |
| 23 | 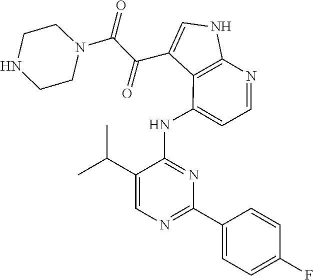 [SMILES: CC(C)c1cnc(-c2ccc(F)cc2)nc1Nc1ccnc2[nH]cc(C(=O)C(=O)N3CCNCC3)c12] | 0.671 | 1.3 |
| 26 | 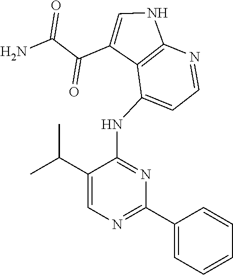 [SMILES: CC(C)c1cnc(-c2ccccc2)nc1Nc1ccnc2[nH]cc(C(=O)C(N)=O)c12] | 0.602 | 0.244 |

Table 4 below illustrates percentage Inhibition of enzyme activity (P38 Alpha MAP kinase) at 100 nM concentration of compounds as well as IC₅₀ value towards ALK-5 inhibition.

| | | Enzyme Activity | |
|---|---|---|---|
| | | MAPK14 | |
| Compound | | (p38alpha) % Inh | ALK5 IC₅₀ |
| No. | Structure | at 100 nM | (μM) |
| 9 | 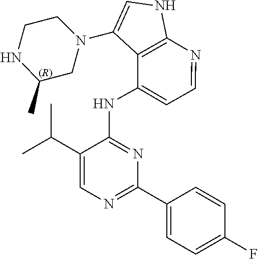 | 52 | 0.103 |
| 10 | 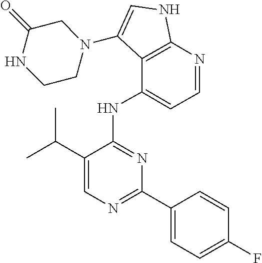 [SMILES: CC(C)c1cnc(-c2ccc(F)cc2)nc1Nc1ccnc2[nH]cc(N3CCNC(=O)C3)c12] | 28 | 0.303 |
| 11 | 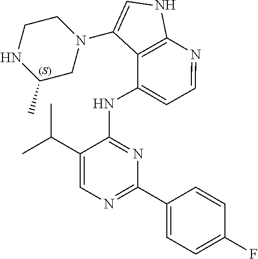 | 18 | 0.147 |
| 12 | 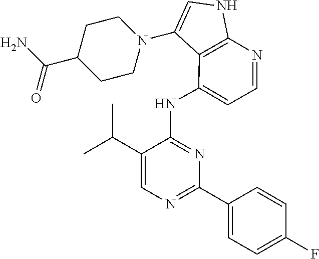 [SMILES: CC(C)c1cnc(-c2ccc(F)cc2)nc1Nc1ccnc2[nH]cc(N3CCC(C(N)=O)CC3)c12] | 37 | 1.17 |
| 27 | 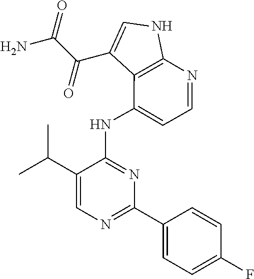 [SMILES: CC(C)c1cnc(-c2ccc(F)cc2)nc1Nc1ccnc2[nH]cc(C(=O)C(N)=O)c12] | 0 | >5 |
| 28 | 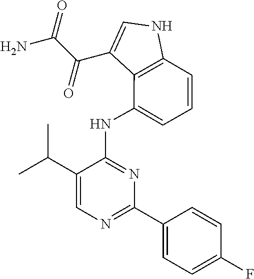 [SMILES: CC(C)c1cnc(-c2ccc(F)cc2)nc1Nc1cccc2[nH]cc(C(=O)C(N)=O)c12] | 21 | >5 |

Table 5 below illustrates compounds exhibiting dual inhibitory activity with their IC₅₀ value towards ALK-5 inhibition.

| Compound No. | Structure | ALK5 IC₅₀ (μM) |
|---|---|---|
| 13 | 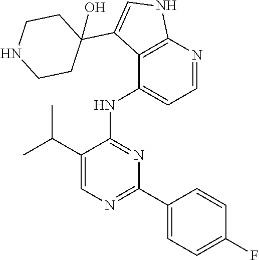 [SMILES: CC(C)c1cnc(-c2ccc(F)cc2)nc1Nc1ccnc2[nH]cc(C3(O)CCNCC3)c12] | 0.108 |
| 14 | 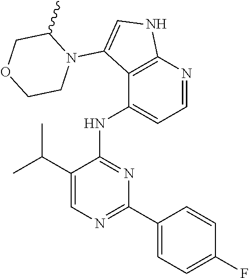 [SMILES: CC(C)c1cnc(-c2ccc(F)cc2)nc1Nc1ccnc2[nH]cc(N3CCOCC3C)c12] | 3.711 |
| 15 | 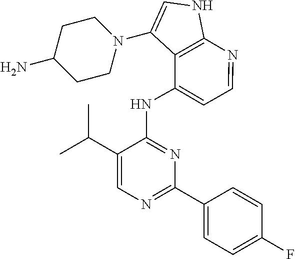 [SMILES: CC(C)c1cnc(-c2ccc(F)cc2)nc1Nc1ccnc2[nH]cc(N3CCC(N)CC3)c12] | 0.245 |
| 16 | 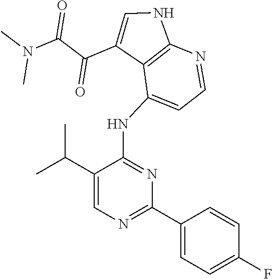 [SMILES: CC(C)c1cnc(-c2ccc(F)cc2)nc1Nc1ccnc2[nH]cc(C(=O)C(=O)N(C)C)c12] | 1.925 |
| 20 | 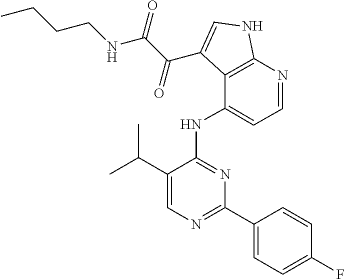 [SMILES: CCCCNC(=O)C(=O)c1c[nH]c2nccc(Nc3nc(-c4ccc(F)cc4)ncc3C(C)C)c12] | >5 |
| 21 | 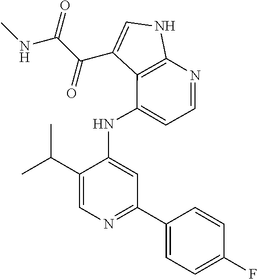 [SMILES: CNC(=O)C(=O)c1c[nH]c2nccc(Nc3cc(-c4ccc(F)cc4)ncc3C(C)C)c12] | 0.574 |
| 22 | 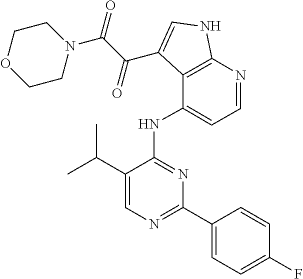 [SMILES: CC(C)c1cnc(-c2ccc(F)cc2)nc1Nc1ccnc2[nH]cc(C(=O)C(=O)N3CCOCC3)c12] | 3.951 |
| 24 | 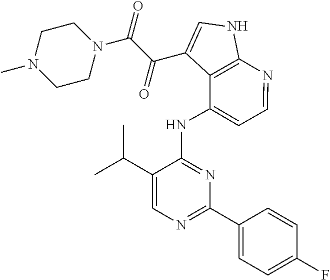 [SMILES: CC(C)c1cnc(-c2ccc(F)cc2)nc1Nc1ccnc2[nH]cc(C(=O)C(=O)N3CCN(C)CC3)c12] | 1.502 |
| 25 | 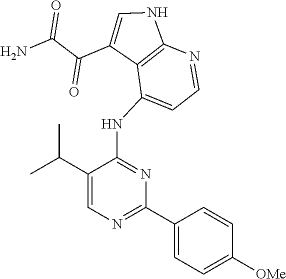 [SMILES: COc1ccc(-c2ncc(C(C)C)c(Nc3ccnc4[nH]cc(C(=O)C(N)=O)c34)n2)cc1] | 2.57 |
| 29 | 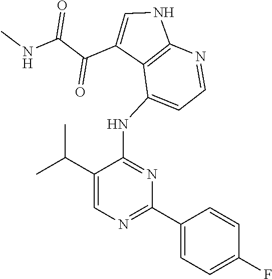 [SMILES: CNC(=O)C(=O)c1c[nH]c2nccc(Nc3nc(-c4ccc(F)cc4)ncc3C(C)C)c12] | 2.772 |

Cell Viability Assay in MIA PaCa-2 and Panc-1

MIA PaCa-2 (ATCC® CRL-1420™) and Panc-1 (CRL-1469™), both pancreatic cell lines were seeded in their respective medium (DMEM, Cat #10569044 with 10% FBS, Cat #16000044; Gibco for Panc-1 and an additional 2.5% Horse Serum, Cat #16050122; Gibco for MIA PaCa-2) at a cell count of 3000 cells per 100 μl per well in a 96 well edge plate (167425; ThermoFisher). Cells were allowed to grow at 37° C. for 24 hr in 5% CO₂ (culture conditions) in a Nuaire incubator (humidified). Serially diluted compounds (100 μl) were added to the culture plate 24 hr later and the cultures (MIA PaCa-2 and Panc-1) were further incubated in culture conditions for 96 hr. Experiment was terminated at the completion of 96 hr exposure to the drug by replacing the medium with 100 μl of 1 mM of resazurin (R7017; Sigma) prepared in culture medium and the plates were further incubated in culture conditions for 4 hr. Fluorescence was then measured using a multimodal plate reader (Biotek Synergy Neo) at an excitation wavelength of 535 nm and emission wavelength of 590 nm to obtain relative fluorescence units. Data analysis was done by subtracting the background fluorescence (only medium blank) value from each reading and then normalizing with the vehicle control (DMSO treated cells) to obtain percent survival/proliferation. Percent survival at different doses was used to calculate IC₅₀ by fitting the curve to the “four-parameter variable slope logistic model” using Prism Graph Pad.

Table 5 below illustrates compounds exhibiting inhibitory activity with their IC₅₀ value towards MIA PaCa-2 and Panc-1 cell lines inhibition.

| | Panc-1_Cell | MiaPaCa-2_Cell |
|---|---|---|
| | Viability_IC₅₀ | Viability_IC₅₀ |
| Compound No. | (μM) | (μM) |
| Compound No-1 | 2.31 | 1.63 |
| Compound No-2 | 2.21 | 7.7 |
| Compound No-3 | >27 | >30 |
| Compound No-4 | 1.88 | 1.54 |
| Compound No-5 | >30 | >30 |
| Compound No-6 | 0.645 | 0.655 |
| Compound No-7 | 2.695 | 2.435 |
| Compound No-8 | >30 | >30 |
| Compound No-9 | 3.28 | 3.53 |
| Compound No-10 | 2.73 | 3.465 |
| Compound No-11 | 2.815 | 2.035 |
| Compound No-12 | 2.6 | 1.775 |
| Compound No-13 | 17.92 | 10.915 |
| Compound No-14 | 11.19 | 11.085 |
| Compound No-15 | 2.965 | 5.5 |
| Compound No-16 | 17.11 | 10.22 |
| Compound No-17 | >30 | 0.765 |
| Compound No-18 | >30 | >30 |
| Compound No-19 | 23.2 | 15.46 |
| Compound No-20 | >30 | >30 |
| Compound No-21 | 12.695 | 10.65 |
| Compound No-22 | 14.23 | >30 |
| Compound No-23 | 5.85 | 1.135 |
| Compound No-24 | >30 | 0.77 |
| Compound No-25 | >30 | >30 |
| Compound No-26 | >30 | 2.195 |
| Compound No-27 | >30 | >30 |
| Compound No-28 | >30 | >30 |
| Compound No-29 | 26 | 23 |

Pharmaceutically acceptable salts are contemplated to be within the scope of the present invention. The compounds of the present invention are bases and salts of such compounds may be formed with acids, for example, a salt with inorganic acid such as hydrochloric acid or a salt with organic acid such as trifluoroacetic acid.

The compounds of this invention may be prepared by employing reactions as shown in the following schemes, in addition to other standard manipulations that are known in the literature or exemplified in the experimental procedures. These schemes, therefore, are not limited by the compounds listed nor by any particular substituents employed for illustrative purposes and in no way limit the scope of the invention.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### Advantages of the Invention

The present disclosure provides dual ALK5/P38α kinase inhibitor compounds.

The present disclosure provides a method of synthesis of compounds that possess dual ALK5/P38α kinase inhibitory activity.

The present disclosure provides method of treatment of conditions associated with excessive activity of any or a combination of ALK5 and P38α kinase.

The present disclosure provides method of treatment of cancer by administering at least one agent that inhibit any or a combination of ALK5 and P38α MAP kinase.

The present disclosure provides method of treatment of inflammatory disease/ailment by administering at least one agent that inhibit any or a combination of ALK5 and P38α MAP kinase.

The present disclosure provides method of treatment of cancer by administering at least one agent that inhibit any or a combination of ALK5 and P38α MAP kinase in combination with at least one immunotherapeutic agent.

The present disclosure provides method of treatment of inflammatory disease by administering at least one agent that inhibit any or a combination of ALK5 and P38α MAP kinase in combination with at least one anti-inflammatory agent.

The present disclosure provides pharmaceutical compositions containing at least one compound that inhibit ALK5 and P38α MAP kinase.

The present disclosure provides pharmaceutical compositions containing at least one compound possessing dual inhibitory activity against ALK5 and P38α MAP kinase that find utility in treatment of cancer.

The present disclosure provides pharmaceutical compositions containing at least one compound possessing dual inhibitory activity against ALK5 and P38α MAP kinase that find utility in treatment of inflammatory disease.

1. A compound of Formula (I),
pharmaceutically acceptable salt, polymorph, solvate or stereoisomer thereof: 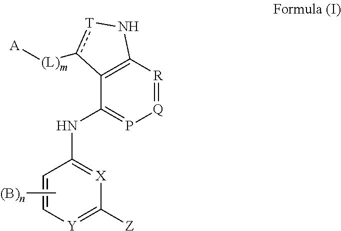
wherein:
L is —C(O)C(O)—;
X and Y independently represent CR₁ or N,
A represents any of: (i) H, —NH₂, —NHR₁, —NR₁R₁, N⁺(O⁻)R₁R₂, —NHC(O)H, —NHC(O)R₁, —NR₁C(O)R₁, —NHC(O)NH2, —NHC(O)NR₁R₂, —NR1C(O)NHR₁, —SR1, —SO₂NR₁R₁, —C(O)NH₂, —C(O)NHR₁, —C(O)NR₁R₂, —CONHSO₂H, —C(O)NHSO₂R₁, —C(O)NR₁SO₂R₁, or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ is independently H, optionally substituted C₁₋₆ linear or branched alkyl group, an optionally substituted C₂₋₆ linear or branched alkenyl group, an optionally substituted C₂₋₆ alkynyl group and an optionally substituted C₃₋₇ cyclic alkyl group;
R₃ is halogen, oxo, —OH, —OR₁, —OCR₁—CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SH, —SR₁, —S(O)H, —S(O)R₁, —SO₂R₁, —SO₂NH₂, —SO₂NHR₁, —SO₂NR₁R₂, CF₃, —CHF₂, —CH₂F, —OCF₃, —OCHF₂, —CN, —CO₂H, —CO₂R₁, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁;
X, Y, P, Q, R, and T independently represents CR₁, or N;
Z represents an optionally substituted aryl or an optionally substituted heteroaryl having up to 12 carbon atoms with one or more heteroatoms independently selected from O, N and S;
B represents H, halogen, Cu, linear or branched alkyl, acyl, C₃₋₈ cycloalkyl, C₁-C₄ halo alkyl, C₁-C₄ halo alkoxy, CHR₂ₐR_{2b}, halogen, —OH, —OR₁, —OCR₂ₐ, wherein R₂ₐ and R_{2b} are independently hydrogen, C₁-C₆ linear or branched alkyl, C₃₋₈ cycloalkyl, optionally substituted aryl, and an optionally substituted heteroaryl group having up to 12 carbon atoms and having one or more heteroatoms in its ring system which are each independently selected from O, N and S, aryl and hetro aryl may be further substituted with R₄;
R₄ represents C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ, —C(O)CH₂OH, —CH(CH₃)₂OH, C(O)R₄ₐR_{4b}OH, wherein R₄ₐ and R_{4b} are independently hydrogen, C₁-C₆ linear or branched alkyl, G-s cycloalkyl, Halo, OH, NH₂, —CONH₂;
n is 0 or an integer from 1-2, and
m is 0 or 1,
with a proviso that at least one of X and Y represents N and when X represents CR1, m≠0.

2. A compound as claimed in claim 1, wherein the compound is a compound of Formula (IA), pharmaceutically acceptable salt, polymorph, solvate or stereoisomer thereof: 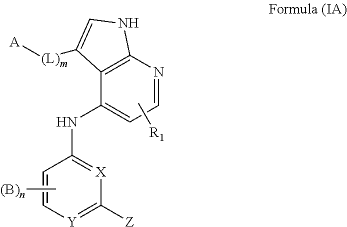
wherein:
L represents —C(O)C(O)—;
X and Y independently represent CR₁ or N;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁, or (ii) cyclic C3-C7 alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted C₁₋₆ linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, OC(O)R₁, —OC(O)NH₂, OC(O)NHR₁, —OC(O)NR₁R₂, —NO₂, —NH₂, —NHR₁, —NR₁R>, N⁺(O⁻)R₁R₂, —NHC(O)H, —NHC(O)R₁,
—NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁;
Z represents an optionally substituted aryl or an optionally substituted heteroaryl having up to 12 carbon atoms with one or more heteroatoms selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-2, and
m is 0 or 1,
with a proviso that at least one of X and Y represents N, and when X represents CR₁, m≠0.

3. The compound as claimed in claim 2, wherein said compound is of any of the compound of Formula (IIA), Formula (III) or Formula (IV), pharmaceutically acceptable salt, polymorph, solvate or stereoisomer thereof: 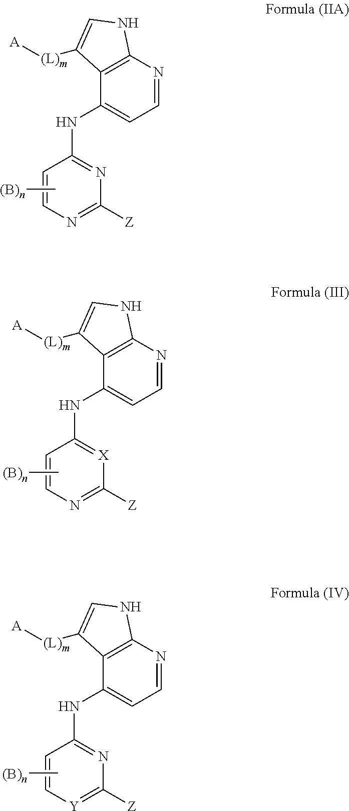
wherein:
L represents —C(O)C(O)—;
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted C₁₋₆ linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo —OH, —OR₁, —OCR₁, —CONR₁, OC(O)R₁, —OC(O)NH₂, OC(O)NHR₁, —OC(O)NR₁R₂, —NO₂, —NH₂, —NHR₁, —NR₁R>, N⁺(O⁻)R₁R₂, —NHC(O)H, —NHC(O)R₁,
—NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁;
X and Y represent CR₁;
Z represents an optionally substituted aryl or an optionally substituted heteroaryl having up to 12 carbon atoms with one or more heteroatoms selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂;
n is 0 or an integer from 1-2, and
m is 0 or 1,
with a proviso that when X represents CR1, m≠0.

4. The compound as claimed in claim 2, wherein said compound is of any of Formula (IIB) or Formula (IVA) Formula (IVB),
pharmaceutically acceptable salt, polymorph, solvate or stereoisomer thereof: 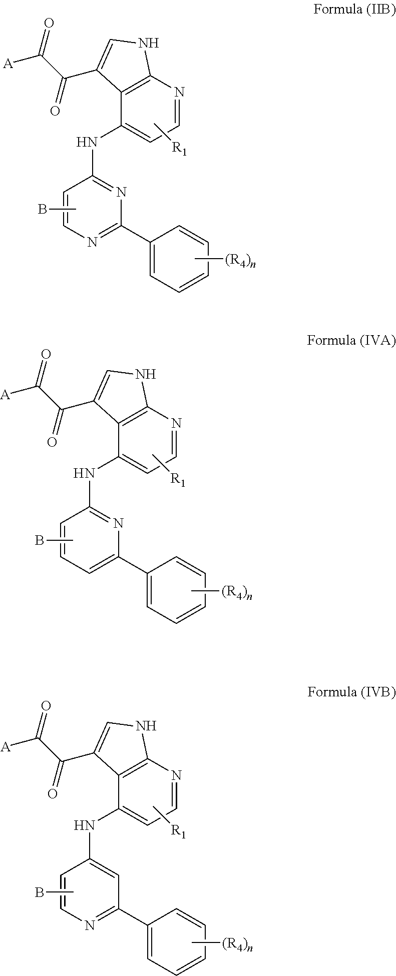
wherein:
A represents any of: (i) H, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SR₁, —SO₂NR₁R₁, —C(O)NH₂, —C(O)NHR₁, —C(O)NR₁R₂, —CONHSO₂H, —C(O)NHSO₂R₁, —C(O)NR₁SO₂R₁; or (ii) cyclic C₃-C₇ alkylamino, imidazolyl, piperazinyl, morpholinyl, thiomorpholinyl, piperidinyl, azepanyl, pyrrolidinyl, triazole and azetidinyl, optionally substituted with any or a combination of R₁, R₂ or R₃;
R₁ and R₂ independently represent H, optionally substituted C₁₋₆ linear or branched alkyl group, an optionally substituted C₂₋₆ linear or branched alkenyl group, an optionally substituted C₂₋₆ alkynyl group, or an optionally substituted C₃₋₇ cyclic alkyl group;
R₃ represents halogen, oxo, —OH, —OR₁, —OCR₁, —CONR₁, OC(O)R₁, —OC(O)NH₂, OC(O)NHR₁, —OC(O)NR₁R>, —NO₂, —NH₂, —NHR₁, —NR₁R₁, N⁺(O⁻)R₁R₂, —NHC(O)H, —NHC(O)R₁,
—NR₁C(O)R₁, —NHC(O)NH₂, —NHC(O)NR₁R₂, —NR₁C(O)NHR₁, —SH, —SR₁, —S(O)H, —S(O)R₁,
—SO₂R₁, —SO₂NH₂, —SO₂NHR₁, —SO₂NR₁R₂, CF₃, —CHF₂, —CH₂F, —OCF₃, —OCHF₂, —CN, —COOH, —COOR₁, —CHO, —C(O)R₁, —C(O)NH₂, —C(O)NHR₁, —C(O)NR₁R>, —CONHSO₂H, —C(O)NHSO₂R₁, —C(O)NR₁SO₂R₁;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, —OH, —OR₁, and —OCR₂ₐ, wherein R₂ₐ and R_{2b} independently represent H, C₁-C₆ linear or branched alkyl, C₃₋₈ cycloalkyl, optionally substituted aryl, and an optionally substituted heteroaryl group having up to 12 carbon atoms with one or more heteroatoms selected from O, N and S, optionally said aryl group and heteroaryl group are substituted with R₄;
R₄ represents any of C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ,
—C(O)CH₂OH, —CH(CH₃)₂OH, and C(O)R₄ₐR_{4b}OH, wherein R₄ₐ and R_{4b} independently represent H, C₁-C₆ linear or branched alkyl, C₃₋₈ cycloalkyl, Halo, OH, NH₂, and —CONH₂; and
n is 0 or an integer from 1-5.

5. The compound as claimed in claim 2, wherein the compound is a compound of Formula (V),
pharmaceutically acceptable salt, polymorph, solvate or stereoisomer thereof: 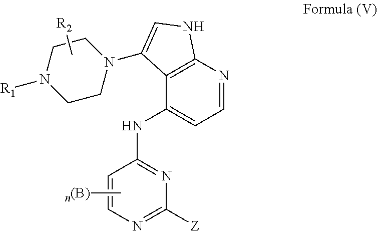
wherein:
R₁ and R₂ is independently H, halogen, oxo, —OH, —OR₁, —OCR₁—CONR₁, OC(O)R₁, —OC(O)NH₂, OC(O)NHR₁, —OC(O)NR₁R₂, —NO₂, —NH₂, —NHR₁, —NR₁R₂, N⁺(O⁻)R₁R₂, —NHC(O)H, —NHC(O)R₁, —NR₁C(O)R₁, —NHC(O)NH₂, —NHC(O)NR₁R₂, —NR₁C(O)NHR₁, —SH, —SR₁, —S(O)H, —S(O)R₁, —SO₂R₁, —SO₂NH₂, —SO₂NHR₁, —SO₂NR₁R₂, CF₃, —CHF₂, —CH₂F, —OCF₃, —OCHF₂, —CN, —CO₂H, —CO₂R₁, —CHO, —C(O)R₁, —C(O)NH₂, —C(O)NHR₁, —C(O)NR₁R₂, —CONHSO₂H, —C(O)NHSO₂R₁, —C(O)NR₁SO₂R₁; optionally substituted CM linear or branched alkyl group, an optionally substituted C₂₋₆ linear or branched alkenyl group, an optionally substituted C₂₋₆ alkynyl group, an optionally substituted C₃₋₇ cyclic alkyl group;
Z represents an optionally substituted aryl or an optionally substituted heteroaryl having up to 12 carbon atoms with one or more heteroatoms independently selected from O, N and S;
B represents H, halogen, C₁₋₆ linear or branched alkyl, acyl, C₃₋₈ cycloalkyl, C₁-C₄ halo alkyl, C₁-C₄ halo alkoxy, CHR₂ₐR_{2b}, halogen, —OH, —OR₁, —OCR₂ₐ, wherein R₂ₐ and R_{2b} are independently hydrogen, C₁-C₆ linear or branched alkyl, C₃₋₈ cycloalkyl, optionally substituted aryl, and an optionally substituted heteroaryl group having up to 12 carbon atoms and having one or more heteroatoms in its ring system which are each independently selected from O, N and S, aryl and hetro aryl may be further substituted with R₄;
R₄ represents C₁₋₆ linear or branched alkyl, acyl, halogen, C₃₋₈ cycloalkyl, C₁-C₄ halo alkyl, C₁-C₄ halo alkoxy, (CH)ₙR₄ₐR_{4b}, —COR₄ₐ, —CONR₄ₐ, OC(O)R₄ₐ, OC(O)NHR₄ₐ, —C(O)CH₂OH, —CH(CH₃)₂OH, C(O)R₄ₐR_{4b}OH, wherein R₄ₐ and R_{4b} are independently hydrogen, C₁-C₆ linear or branched alkyl, C₃₋₈ cycloalkyl, Halo, OH, NH₂, —CONH₂; and
n is 0 or an integer from 1-2.

6. The compound as claimed in claim 2, wherein the compound is a compound selected from the group consisting of:
N-(2-(4-fluorophenyl)-5-isopropylpyrimidin-4-yl)-3-(piperazin-1-yl)-1H-pyrrolo [2, 3-b]pyridin-4-amine;
N-(2-(4-fluorophenyl)-5-isopropylpyrimidin-4-yl)-3-(4-methylpiperazin-1-yl)-1H-pyrrolo [2, 3-b] pyridin-4-amine;
N-(2-(4-fluorophenyl)-5-isopropylpyrimidin-4-yl)-3-(piperidin-1-yl)-1H-pyrrolo [2, 3-b]pyridin-4-amine;
N-(2-(4-fluorophenyl)-5-isopropylpyrimidin-4-yl)-3-(2-methylpiperazin-1-yl)-1H-pyrrolo[2,3-b]pyridin-4-amine;
N-(2-(4-fluorophenyl)-5-isopropylpyrimidin-4-yl)-3-morpholino-1H-pyrrolo [2, 3-b]pyridin-4-amine;
N-(2-(4-fluorophenyl)-5-isopropylpyrimidin-4-yl)-3-(1H-1, 2,4-triazol-1-yl)-1H-pyrrolo [2, 3-b] pyridin-4-amine;
(S)—N-(2-(4-fluorophenyl)-5-isopropylpyrimidin-4-yl)-3-(2-methylpiperazin-1-yl)-1H-pyrrolo[2,3-b]pyridin-4-amine;
(R)—N-(2-(4-fluorophenyl)-5-isopropylpyrimidin-4-yl)-3-(2-methylpiperazin-1-yl)-1H-pyrrolo[2,3-b]pyridin-4-amine;
(R)—N-(2-(4-fluorophenyl)-5-isopropylpyrimidin-4-yl)-3-(3-methylpiperazin-1-yl)-1H-pyrrolo[2,3-b]pyridin-4-amine;
4-(4-(2-(4-fluorophenyl)-5-isopropylpyrimidin-4-ylamino)-1H-pyrrolo [2, 3-b] pyridin-3-yl) piperazin-2-one;
(S)—N-(2-(4-fluorophenyl)-5-isopropylpyrimidin-4-yl)-3-(3-methylpiperazin-1-yl)-1H-pyrrolo[2,3-b]pyridin-4-amine;
1-(4-((2-(4-fluorophenyl)-5-isopropylpyrimidin-4-yl)amino)-1H-pyrrolo[2,3-b]pyridin-3-yl)piperidine-4-carboxamide;
4-(4-((2-(4-fluorophenyl)-5-isopropylpyrimidin-4-yl)amino)-1H-pyrrolo[2,3-b]pyridin-3-yl)piperidin-4-ol;
N-(2-(4-fluorophenyl)-5-isopropylpyrimidin-4-yl)-3-(3-methylmorpholino)-1H-pyrrolo [2, 3-b] pyridin-4-amine;
3-(4-aminopiperidin-1-yl)-N-(2-(4-fluorophenyl)-5-isopropylpyrimidin-4-yl)-1H-pyrrolo[2,3-b]pyridin-4-amine;
2-(4-((2-(4-fluorophenyl)-5-isopropylpyrimidin-4-yl) amino)-1H-pyrrolo[2,3-b]pyridin-3-yl)-N,N-dimethyl-2-oxoacetamide;
2-(4-((2-(4-fluorophenyl)-5-isopropylpyrimidin-4-yl)amino)-1H-pyrrolo[2,3-b]pyridin-3-yl)-2-oxoacetamide;
2-(4-((2-(5-chloro-2-fluorophenyl)-5-isopropylpyrimidin-4-yl)amino)-1H-pyrrolo[2,3-b]pyridin-3-yl)-N-methyl-2-oxoacetamide;
2-(4-((2-(4-fluorophenyl)-5-isopropylpyridin-4-yl)amino)-1H-pyrrolo[2,3-b]pyridin-3-yl)-2-oxoacetamide;
N-butyl-2-(4-(2-(4-fluorophenyl)-5-isopropylpyrimidin-4-ylamino)-1H-pyrrolo [2, 3-b]pyridin-3-yl)-2-oxoacetamide;
2-(4-((2-(4-fluorophenyl)-5-isopropylpyridin-4-yl)amino)-1H-pyrrolo[2,3-b]pyridin-3-yl)-N-methyl-2-oxoacetamide;
1-(4-(2-(4-fluorophenyl)-5-isopropylpyrimidin-4-ylamino)-1H-pyrrolo[2, 3-b] pyridin-3-yl)-2-morpholinoethane-1,2-dione;
1-(4-(2-(4-fluorophenyl)-5-isopropylpyrimidin-4-ylamino)-1H-pyrrolo[2,3-b]pyridin-3-yl)-2-(piperazin-1-yl)ethane-1,2-dione;
1-(4-(2-(4-fluorophenyl)-5-isopropylpyrimidin-4-ylamino)-1H-pyrrolo [2, 3-b] pyridin-3-yl)-2-(4-methylpiperazin-1-yl) ethane-1, 2-dione;
2-(4-((5-isopropyl-2-(4-methoxyphenyl) pyrimidin-4-yl) amino)-1H-pyrrolo[2,3-b]pyridin-3-yl)-2-oxoacetamide;
2-(4-((5-isopropyl-2-phenylpyrimidin-4-yl)amino)-1H-pyrrolo[2,3-b]pyridin-3-yl)-2-oxoacetamide;
2-(4-(6-(4-fluorophenyl)-3-isopropylpyridin-2-ylamino)-1H-pyrrolo [2, 3-b] pyridin-3-yl)-2-oxoacetamide;
2-(4-((2-(4-fluorophenyl)-5-isopropylpyrimidin-4-yl)amino)-1H-pyrrolo[2,3-b]pyridin-3-yl)-N-methyl-2-oxoacetamide,
or pharmaceutically acceptable salt, polymorph, solvate or stereoisomer thereof.

7. A method of treating disease associated with excessive level of any or a combination of ALK5 and P38α MAP kinase in an individual in need thereof comprising administering to the individual a therapeutically effective amount of a compound of claim 2, or a pharmaceutically acceptable salt thereof.

8. A method of treating disease associated with any or a combination of excessive level of ALK5 and P38α MAP kinase in an individual in need thereof comprising administering to the individual a therapeutically effective amount of a compound of claim 2 in combination with one or more immunotherapeutic agents.

9. (canceled)

10. A pharmaceutical composition comprising a compound of claim 2, or a tautomer, salt, polymorph, solvate or stereoisomer thereof, and a pharmaceutically acceptable carrier.

11. A pharmaceutical composition comprising a compound of claim 2, one or more immunotherapeutic agent and a pharmaceutically acceptable carrier.

12. (canceled)

13. Use of a compound of claim 2, or a pharmaceutically acceptable salt or solvate thereof, in the manufacture of a medicament for treatment of a disease mediated by ALK5 and P38α MAP kinase.

14. A kit comprising a compound of claim 2, or a salt, polymorph, solvate or stereoisomer thereof.

15. The compound of claim 2, wherein the compound is selected from Compound Nos. 1 to 55 in table 1 or a salt, polymorph, solvate or stereoisomer thereof.

16. A method of treating inflammatory disease or proliferative disease in an individual in need thereof comprising administering to the individual a therapeutically effective amount of a compound of the claim 2, or a pharmaceutically acceptable salt thereof.
